(19) United States
(12) Patent Application Publication
(10) Pub. No.: US 2022/0249522 A1
(43) Pub. Date: Aug. 11, 2022

(54) Hypoxia Targeting Compositions and Combinations Thereof with a PARP Inhibitor and Methods of Use Thereof

(71) Applicant: The Board of Trustees of the Leland Stanford Junior University, Stanford, CA (US)

(72) Inventors: Manal Mehibel, Stanford, CA (US); Amato J. Giaccia, Stanford, CA (US)

(73) Assignee: The Board of Trustees of the Leland Stanford Junior University, Stanford, CA (US)

(21) Appl. No.: 17/311,673

(22) PCT Filed: Dec. 6, 2019
(86) PCT No.: PCT/US19/65065

§ 371 Date: Jun. 7, 2021

Related U.S. Application Data
(60) Provisional application No. 62/777,001, filed on Dec. 7, 2018.

(51) Int. Cl. A61K 31/675; A61K 31/404; A61K 31/136; A61K 31/4168; A61K 31/5377; A61K 31/454; A61K 31/407; A61K 31/661; A61K 31/519; A61K 31/53; A61K 31/166; A61K 31/496; A61K 31/502; A61K 31/55; A61K 31/5025; A61K 31/4184; A61P 35/00

(52) U.S. Cl. CPC A61K 31/675; A61P 35/00; A61K 31/136; A61K 31/4168; A61K 31/5377; A61K 31/454; A61K 31/407; A61K 31/661; A61K 31/519; A61K 31/53; A61K 31/166; A61K 31/496; A61K 31/502; A61K 31/55; A61K 31/5025; A61K 31/4184; A61K 31/404

(57) ABSTRACT

Methods for treating a cancer are provided, the methods comprising administering to an individual an effective amount of a hypoxia targeting composition, such as a hypoxia-activated drug or a prodrug thereof, and combinations thereof with an effective amount of a poly(ADP-ribose) polymerase (PARP) inhibitor. In some instances, one or more of a homology recombination (HR) efficiency status, an IDH mutation status, and a hypoxia status of a cancer is used as a basis for selecting an individual for a treatment disclosed herein. Also provided are compositions (such as pharmaceutical formulations), medicine, kits, and unit dosages useful for the methods described herein.

### CROSS REFERENCE TO RELATED APPLICATIONS

This application claims priority to U.S. Provisional Application Ser. No. 62/777,001, filed Dec. 7, 2018, which is incorporated herein by reference in its entirety.

### TECHNICAL FIELD

The present disclosure provides methods for treating a cancer comprising administering to an individual: (i) an effective amount of a hypoxia targeting composition (such as a hypoxia-activated drug or a prodrug thereof); and (ii) an effective amount of a poly(ADP-ribose) polymerase (PARP) inhibitor. The present disclosure also provides, in other aspects, methods for treating a cancer comprising administering to an individual an effective amount of a hypoxia targeting composition (such as a hypoxia-activated drug or a prodrug thereof). Also provided are kits, medicines, and compositions (such as pharmaceutical formulations) useful for the methods described herein.

### BACKGROUND

Hypoxia targeting compositions are a class of drugs that have selective toxic effects in hypoxic conditions via, e.g., chemical mechanisms and/or selective targeting. For example, hypoxia-activated drugs or prodrugs thereof are compounds that are chemically converted, such as via bioreduction, to a toxic form thereof and without sufficient oxygen cause cellular damage. See Misty, I. N. et al., Int J Radiat Oncol Biol Phys, 98, 2017, which is hereby incorporated by reference in its entirety. Tirapazamine, a hypoxia targeting composition, and more specifically a hypoxia-activated drug, is an aromatic heterocycle di-N-oxide that is activated via reduction by cellular enzymes to a radical intermediate. In the presence of a sufficient oxygen concentration, this radical intermediate is converted back to the non-toxic starting material, tirapazamine (a process known as futile redox-cycling). In contrast, in a hypoxic environment, activated tirapazamine results in DNA damage, such as DNA strand cleavage. Despite the selective toxicity of tirapazamine in hypoxic conditions and that such hypoxic conditions are present in many difficult to treat tumors, e.g., solid tumors, tirapazamine has demonstrated a lack of efficacy in clinical trials for treating cancers, such as non-small cell lung cancer and head and neck cancer.

All references cited herein, including patent applications and publications, are incorporated by reference in their entirety.

### BRIEF SUMMARY

In one aspect, the present application provides methods for treating a cancer in an individual in need thereof, the method comprising administering to the individual (i) an effective amount of a hypoxia targeting composition (such as a hypoxia-activated drug or a prodrug thereof), and (ii) an effective amount of a poly(ADP-ribose) polymerase (PARP) inhibitor.

In some embodiments, the hypoxia targeting composition is a hypoxia-activated drug or a prodrug thereof.

In some embodiments, the hypoxia-activated drug or the prodrug thereof is selected from the group consisting of: apaziquone, AQ4N, etanidazole, evofosfamide, nimorazole, pimonidazole, porfiromycin, PR-104, tarloxotinib, and tirapazamine, or an analog or derivative thereof.

In some embodiments, the effective amount of the hypoxia targeting composition (such as the hypoxia-activated drug or the prodrug thereof) is about 0.1 mg to 1000 mg.

In some embodiments, the effective amount of the hypoxia targeting composition (such as the hypoxia-activated drug or the prodrug thereof) is suitable for oral administration.

In some embodiments, the PARP inhibitor is selected from the group consisting of: 3-aminobenzamine, BGD-290, CEP 9722, E7016, iniparib, niraparib, olaparib, rucaparib, talazoparib, Fluzoparib, and veliparib.

In some embodiments, the effective amount of the PARP inhibitor is about 20 mg to about 2000 mg.

In some embodiments, the individual is not responsive to the effective amount of the PARP inhibitor when administered alone. In some embodiments, the individual is resistant or refractory to the effective amount of the PARP inhibitor when administered alone. In some embodiments, the individual is only partially responsive and not adequately responsive to an effective amount of PARP inhibitor when administered alone.

In some embodiments, the effective amount of the hypoxia targeting composition (such as the hypoxia-activated drug or the prodrug thereof), and the effective amount of the PARP inhibitor are administered simultaneously. In some embodiments, the effective amount of the hypoxia targeting composition (such as the hypoxia-activated drug or the prodrug thereof), and the effective amount of the PARP inhibitor are administered sequentially. In some embodiments, the effective amount of the hypoxia targeting composition (such as the hypoxia-activated drug or the prodrug thereof), and the effective amount of the PARP inhibitor are administered concurrently.

In some embodiments, the homologous recombination (HR) deficiency status of the cancer is used as a basis for selecting the individual for treatment. In some embodiments, the HR deficiency status of the cancer is based on a homologous recombination (HR) deficiency signature. In some embodiments, the HR deficiency status of the cancer is based on one or more of the following: (i) a gene sequence, or a product thereof, or an expression level thereof; (ii) loss of heterozygosity (LOH); (iii) telomeric allelic imbalance (TAI); (iv) large-scale state transitions (LST); and (v) promoter methylation. In some embodiments, the HR deficiency status of the cancer is determined based on one or more of the following: (i) assessing a gene sequence, or a product thereof, or an expression level thereof; (ii) assessing loss of heterozygosity (LOH); (iii) assessing telomeric allelic imbalance (TAI); (iv) assessing large-scale state transitions (LST); and (v) assessing promoter methylation. In some embodiments, the HR deficiency status of the cancer is based on one or more of: DNA sequencing, RNA sequencing, and protein sequencing.

In some embodiments, the HR deficiency status of the cancer is determined prior to administration of (i) the effective amount of the hypoxia targeting composition (such as the hypoxia-activated drug or the prodrug thereof), and (ii) the effective amount of the PARP inhibitor.

In some embodiments, the methods further comprise determining the HR deficiency status of the cancer prior to administration of (i) the effective amount of the hypoxia targeting composition (such as the hypoxia-activated drug or the prodrug thereof), and (ii) the effective amount of the PARP inhibitor.

In some embodiments, the methods further comprise selecting the individual for treatment based on the HR deficiency status of the cancer.

In some embodiments, the IDH mutation status of the cancer is used as a basis for selecting the individual for treatment. In some embodiments, the IDH mutation status is based on an IDH mutation. In some embodiments, the IDH mutation status of the cancer is based on one or more of the following: (i) a gene sequence, or a product thereof, of IDH1 and/or IDH2; (ii) a change in an activity level of IDH1 and/or IDH2; and (iii) a level of a metabolic biomarker. In some embodiments, the IDH mutation status of the cancer is determined based on one or more of the following: (i) assessing a gene sequence, or a product thereof, of IDH1 and/or IDH2; (ii) assessing a change in an activity level of IDH1 and/or IDH2; and (iii) assessing a level of a metabolic biomarker.

In some embodiments, the IDH mutation status of the cancer is determined prior to administration of (i) the effective amount of the hypoxia targeting composition (such as the hypoxia-activated drug or the prodrug thereof), and (ii) the effective amount of the PARP inhibitor.

In some embodiments, the methods further comprise determining the IDH mutation status of the cancer prior to administration of (i) the effective amount of the hypoxia targeting composition (such as the hypoxia-activated drug or the prodrug thereof), and (ii) the effective amount of the PARP inhibitor.

In some embodiments, the methods further comprise selecting the individual for treatment based on the IDH mutation status of the cancer.

In some embodiments, the hypoxia status of the cancer is used as a basis for selecting the individual for treatment. In some embodiments, the hypoxia status of the cancer is based on a low tissue oxygenation level. In some embodiments, the low tissue oxygenation level is a tissue oxygenation level of about 4% or less of oxygen. In some embodiments, the hypoxia status of the cancer is based on one or more of the following: (i) tissue oxygenation level; and (ii) a hypoxia biomarker. In some embodiments, the hypoxia status of the cancer is determined based on one or more of the following: (i) assessing tissue oxygenation level using an oxymetric technique; and (ii) assessing a hypoxia biomarker.

In some embodiments, the hypoxia status of the cancer is determined prior to administration of (i) the effective amount of the hypoxia targeting composition (such as the hypoxia-activated drug or the prodrug thereof), and (ii) the effective amount of the PARP inhibitor.

In some embodiments, the methods further comprise selecting the individual for treatment based on the hypoxia status of the cancer.

In another aspect, the present application provides methods for treating a cancer in an individual in need thereof, the method comprising administering to the individual an effective amount of a hypoxia-activated drug or a prodrug thereof, wherein a homologous recombination (HR) deficiency status of the cancer is used as a basis for selecting the individual for treatment.

In some embodiments, the hypoxia targeting composition is a hypoxia-activated drug or a prodrug thereof.

In some embodiments, the hypoxia-activated drug or the prodrug thereof is selected from the group consisting of: apaziquone, AQ4N, etanidazole, evofosfamide, nimorazole, pimonidazole, porfiromycin, PR-104, tarloxotinib, and tirapazamine, or an analog or derivative thereof.

In some embodiments, the effective amount of the hypoxia targeting composition (such as the hypoxia-activated drug or the prodrug thereof) is about 0.1 mg to 1000 mg.

In some embodiments, the effective amount of the hypoxia targeting composition (such as the hypoxia-activated drug or the prodrug thereof) is suitable for oral administration.

In some embodiments, the HR deficiency status of the cancer is based on a HR deficiency signature. In some embodiments, the HR deficiency status of the cancer is based on one or more of the following: (i) a gene sequence, or a product thereof, or an expression level thereof; (ii) loss of heterozygosity (LOH); (iii) telomeric allelic imbalance (TAI); (iv) large-scale state transitions (LST); and (v) promoter methylation. In some embodiments, the HR deficiency status of the cancer is determined based on one or more of the following: (i) assessing a gene sequence, or a product thereof, or an expression level thereof; (ii) assessing loss of heterozygosity (LOH); (iii) assessing telomeric allelic imbalance (TAI); (iv) assessing large-scale state transitions (LST); and (v) assessing promoter methylation. In some embodiments, the HR deficiency status of the cancer is based on one or more of: DNA sequencing, RNA sequencing, and protein sequencing.

In some embodiments, the HR deficiency status of the cancer is determined prior to administration of the effective amount of the hypoxia targeting composition (such as the hypoxia-activated drug or the prodrug thereof).

In some embodiments, the methods further comprise determining the HR deficiency status of the cancer prior to administration of the effective amount of the hypoxia targeting composition (such as the hypoxia-activated drug or the prodrug thereof).

In some embodiments, the methods further comprise selecting the individual for treatment based on the HR deficiency status of the cancer.

In another aspect, the present application provides methods for treating a cancer in an individual in need thereof, the method comprising administering to the individual an effective amount of a hypoxia targeting composition (such as a hypoxia-activated drug or a prodrug thereof), wherein an IDH mutation status of the cancer is used as a basis for selecting the individual for treatment.

In some embodiments, the hypoxia targeting composition is a hypoxia-activated drug or a prodrug thereof.

In some embodiments, the hypoxia-activated drug or the prodrug thereof is selected from the group consisting of: apaziquone, AQ4N, etanidazole, evofosfamide, nimorazole, pimonidazole, porfiromycin, PR-104, tarloxotinib, and tirapazamine, or an analog or derivative thereof.

In some embodiments, the effective amount of the hypoxia targeting composition (such as the hypoxia-activated drug or the prodrug thereof) is about 0.1 mg to 1000 mg.

In some embodiments, the effective amount of the hypoxia targeting composition (such as the hypoxia-activated drug or the prodrug thereof) is suitable for oral administration.

In some embodiments, the IDH mutation status is based on an IDH mutation. In some embodiments, the IDH mutation status of the cancer is based on one or more of the following: (i) a gene sequence, or a product thereof, of IDH1 and/or IDH2; (ii) a change in an activity level of IDH1 and/or IDH2; and (iii) a level of a metabolic biomarker. In some embodiments, the IDH mutation status of the cancer is determined based on one or more of the following: (i) assessing a gene sequence, or a product thereof, of IDH1 and/or IDH2; (ii) assessing a change in an activity level of IDH1 and/or IDH2; and (iii) assessing a level of a metabolic biomarker.

In some embodiments, the methods further comprise determining the IDH mutation status of the cancer prior to administration of the effective amount of the hypoxia targeting composition (such as the hypoxia-activated drug or the prodrug thereof).

In some embodiments, the methods further comprise selecting the individual for treatment based on the IDH mutation status of the cancer.

In another aspect, the present application provides methods for treating a cancer in an individual in need thereof, the method comprising administering to the individual an effective amount of a hypoxia targeting composition (such as a hypoxia-activated drug or a prodrug thereof), wherein a hypoxia status of the cancer is used as a basis for selecting the individual for treatment.

In some embodiments, the hypoxia targeting composition is a hypoxia-activated drug or a prodrug thereof.

In some embodiments, the hypoxia-activated drug or the prodrug thereof is selected from the group consisting of: apaziquone, AQ4N, etanidazole, evofosfamide, nimorazole, pimonidazole, porfiromycin, PR-104, tarloxotinib, and tirapazamine, or an analog or derivative thereof.

In some embodiments, the effective amount of the hypoxia targeting composition (such as the hypoxia-activated drug or the prodrug thereof) is about 0.1 mg to 1000 mg.

In some embodiments, the effective amount of the hypoxia targeting composition (such as the hypoxia-activated drug or the prodrug thereof) is suitable for oral administration.

In some embodiments, the hypoxia status of the cancer is based on a low tissue oxygenation level. In some embodiments, the low tissue oxygenation level is a tissue oxygenation level of about 4% or less of oxygen. In some embodiments, the hypoxia status of the cancer is based on one or more of the following: (i) tissue oxygenation level; and (ii) a hypoxia biomarker. In some embodiments, the hypoxia status of the cancer is determined based on one or more of the following: (i) assessing tissue oxygenation level using an oxymetric technique; and (ii) assessing a hypoxia biomarker.

In some embodiments, the hypoxia status of the cancer is determined prior to administration of the effective amount of the hypoxia targeting composition (such as the hypoxia-activated drug or the prodrug thereof).

In some embodiments, the methods further comprise selecting the individual for treatment based on the hypoxia status of the cancer.

In some embodiments, the cancer is a solid tumor. In some embodiments, the cancer is a hematopoietic malignancy. In some embodiments, the cancer is a breast cancer, ovarian cancer, pancreatic cancer, fibrosarcoma, head and neck cancer, prostate cancer, glioma, or acute myeloid leukemia.

In some embodiments, the individual is human.

In another aspect, the present application provides kits comprising: (i) a hypoxia-activated drug or a prodrug thereof, and (ii) a poly(ADP-ribose) polymerase (PARP) inhibitor.

### BRIEF DESCRIPTION OF THE DRAWINGS

FIGS. 1A-1D show plots of survival fraction versus olaparib (AZD-2281) concentration for SUM149 (FIG. 1A), CAPAN-1 (FIG. 1B), HT1080 (FIG. 1C), and OVCAR8 (FIG. 1D) cell lines cultured in 21% oxygen or 2% oxygen conditions.

FIGS. 2A-2D show plots of survival fraction versus talazoparib (BMN 673) concentration for SUM149 (FIG. 2A), CAPAN-1 (FIG. 2B), HT1080 (FIG. 2C), and OVCAR8 (FIG. 2D) cell lines cultured in 21% oxygen or 2% oxygen conditions.

FIG. 3 shows a histogram of survival fractions of OVCAR8 cells treated with a vehicle, olaparib (AZD-2281; 1 μM), or talazoparib (BMN 673; 10 nM) cultured in 21% oxygen, 5% oxygen, or 2% oxygen conditions.

FIGS. 4A-4C show plots of percentage of SUM149 cells with more than 10 γH2AX foci per nucleus after 48 h of PARPi treatment in normoxic (21% oxygen) or hypoxic (2% oxygen) culture conditions (FIG. 4A), representative images of γH2AX foci analyzed by high-throughput microscopy (FIG. 4B), and immunoblots of DDR proteins in SUM149 cells treated with vehicle or PARPi for 48 h in normoxia or hypoxia (FIG. 4C).

FIGS. 5A and 5B show western blot analyzes of the levels of poly(ADP-ribose) (PAR) in OVCAR8 cells (FIG. 5A) and HT1080 cells (FIG. 5B) following treatment with a vehicle, olaparib (AZD-2281; 1 μM), or talazoparib (BMN 673; 10 nM) cultured in 21% oxygen or 2% oxygen conditions.

FIGS. 6A and 6B show histograms of relative PARP activity in OVCAR8 cells (FIG. 6A) treated with a vehicle, olaparib (AZD-2281; 1 μM), or talazoparib (BMN 673; 10 nM), and in SUM149 cells (FIG. 6B) treated with a vehicle, olaparib (AZD-2281; 0.1 μM), or talazoparib (BMN 673; 1 nM) cultured in 21% oxygen or 2% oxygen conditions.

FIGS. 7A-7D show a schematic of diagram of olaparib treatment in OVCAR8 xenografts (FIG. 7A), a Western blot analysis of PAR levels in tumor lysates (FIG. 7B), immunohistochemical staining of vehicle- and olaparib-treated tumors (FIG. 7C), and sensitivity to olaparib inversely correlates with hypoxia in Breast PDX models (FIG. 7D).

FIGS. 8A and 8B show survival percentage of OVCAR8 cells treated with olaparib (0.001-10 μM) and TPZ (0.1-50 μM) or BMN673 (0.01-20 nM) and TPZ (0.1-50 μM) (FIG. 8A), and HSA synergism analysis of OVCAR8 cells treated with varying doses of TPZ and olaparib or TPZ and BMN673 (FIG. 8B).

FIGS. 9A and 9B show survival percentage of SUM149 cells treated with olaparib (0.001-10 μM) and TPZ (0.1-50 μM) or BMN673 (0.01-20 nM) and TPZ (0.1-50 μM) (FIG. 9A), and HSA synergism analysis of SUM149 cells treated with varying doses of TPZ and olaparib or TPZ and BMN673 (FIG. 9B).

FIGS. 10A-10C show histograms of survival fractions of OVCAR8 cells treated with a vehicle, tirapazamine, olaparib, olaparib plus tirapazamine, talazoparib, or talazoparib plus tirapazamine.

FIGS. 11A-11C show histograms of survival fractions of SUM149 cells treated with a vehicle, tirapazamine, olaparib, olaparib plus tirapazamine, talazoparib, or talazoparib plus tirapazamine.

FIGS. 12A and 12B show plots of tumor volume versus days of a treatment regimen. FIG. 12A shows tumor volumes of OVCAR8 xenografts following treatments with a vehicle, olaparib, tirapazamine, or olaparib plus tirapazamine. FIG. 12B shows tumor volumes of SUM149 xenografts following treatments with a vehicle, talazoparib (BMN 673), tirapazamine, or talazoparib plus tirapazamine.

FIG. 12C shows a plot of tumor volume of HT1080 xenografts versus days of a treatment regimen of vehicle, tirapazamine, olaparib, or olaparib plus tirapazamine.

### DETAILED DESCRIPTION

The present application provides, in some aspects, methods for treating a cancer in an individual in need thereof, the methods comprising administering to the individual an effective amount of a hypoxia targeting composition (such as a hypoxia-activated drug or a prodrug thereof). Also provided, in another aspect of the present application, are methods of combination treatments for treating a cancer in an individual in need thereof, the methods comprising administering to the individual (i) an effective amount of a hypoxia targeting composition (such as a hypoxia-activated drug or a prodrug thereof), and (ii) an effective amount of a poly(ADP-ribose) polymerase (PARP) inhibitor. In other aspects of the present application, any one or more of a homologous recombination (HR) status, an isocitrate dehydrogenase (IDH) mutation status, and a hypoxia status of a cancer is used as a basis for selecting an individual for treatment with any of the methods disclosed herein.

The present application is based, in part, on the unexpected finding that a combination comprising a drug having toxic effects in hypoxic conditions, namely, tirapazamine, plus a PARP inhibitor (olaparib or talazoparib) significantly delayed tumor growth in xenografts, as compared to single agent treatments with tirapazamine or a PARP inhibitor or a vehicle control. Such unexpected findings are underscored by the additional findings disclosed herein that HR deficient cell lines and cell lines comprising IDH mutations cultured in hypoxic conditions showed insensitivity to PARP inhibitors when administered alone, and that HR proficient cell lines also showed insensitivity to PARP inhibitors in both normoxic and hypoxic conditions. These findings provide the basis for the improved methods of treating a cancer in an individual described in various aspects herein, including methods comprising administering to the individual an effective amount of a hypoxia targeting composition (such as a hypoxia-activated drug or a prodrug thereof), and combinations thereof also comprising administering an effective amount of a PARP inhibitor, wherein, optionally, use of the improved methods is based on use of patient selection criteria.

Methods described herein exemplified by use of a hypoxia-activated drug or prodrug thereof are not intended to be a limitation of the scope of agents that are useful for the methods disclosed herein. As discussed above, the unexpected findings are based on a drug having toxic effects in hypoxic cells, and it is contemplated that such finding supports the methods disclosed herein, wherein the methods comprise administrating a drug having selective toxicity in a hypoxic environment, including hypoxia targeting compositions, hypoxia-activated drug or prodrugs thereof, and hypoxia cytotoxins.

In one aspect, there is provided methods for treating a cancer in an individual in need thereof, the method comprising administering to the individual an effective amount of a hypoxia targeting composition (such as a hypoxia-activated drug or a prodrug thereof), wherein a HR deficiency status of the cancer is used as a basis for selecting the individual for treatment.

In another aspect, there is provided methods for treating a cancer in an individual in need thereof, the method comprising administering to the individual an effective amount of a hypoxia targeting composition (such as a hypoxia-activated drug or a prodrug thereof), wherein an IDH mutation status of the cancer is used as a basis for selecting the individual for treatment.

In another aspect, there is provided methods for treating a cancer in an individual in need thereof, the method comprising administering to the individual an effective amount of a hypoxia targeting composition (such as a hypoxia-activated drug or a prodrug thereof), wherein a hypoxia status of the cancer is used as a basis for selecting the individual for treatment.

In another aspect, there is provided methods for treating a cancer in an individual in need thereof, the method comprising administering to the individual (i) an effective amount of a hypoxia targeting composition (such as a hypoxia-activated drug or a prodrug thereof), and (ii) an effective amount of a PARP inhibitor. In some embodiments of the combination treatments disclosed herein, the HR deficiency status of the cancer is used as a basis for selecting the individual for treatment. In some embodiments of the combination treatments disclosed herein, the IDH mutation status of the cancer is used as a basis for selecting the individual for treatment. In some embodiments of the combination treatments disclosed herein, the hypoxia status of the cancer is used as a basis for selecting the individual for treatment.

Also provided are kits, medicines, and compositions (such as pharmaceutical compositions and unit dosages) useful for the methods described herein.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

The term “treating” or “treatment,” as used herein, is an approach for obtaining beneficial or desired results including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (e.g., partial or total) of the disease, decreasing the dose of one or more other medications required to treat the disease, delaying the progression of the disease, increasing the quality of life, and/or prolonging survival. Also encompassed by “treating” or “treatment” is a reduction of pathological consequence of the cancer. The methods of the present application contemplate any one or more of these aspects of treatment.

The term “combination therapy” or “combination treatment,” as used herein, is meant that a first agent be administered in conjunction with at least one other agent. “In conjunction with” refers to administration of one treatment modality, such as a hypoxia targeting composition (such as a hypoxia-activated drug or a prodrug thereof), in addition to, but not necessarily at the same time as, administration of another treatment modality, such as a PARP inhibitor. As such, “in conjunction with” refers to administration of one treatment modality before, during, or after delivery of the other treatment modality to the individual.

The term “effective amount,” as used herein, refers to an amount of a compound or composition sufficient to treat a specified disorder, condition, or disease, such as ameliorate, palliate, lessen, and/or delay one or more symptoms of the disorder, condition, or disease. In reference to cancer, an effective amount comprises an amount sufficient to, e.g., cause a tumor to shrink and/or to decrease the growth rate of the tumor (such as to suppress tumor growth) or to prevent or delay other unwanted cell proliferation in the cancer. In some embodiments, an effective amount is an amount sufficient to delay development of cancer. In some embodiments, an effective amount is an amount sufficient to prevent or delay recurrence. An effective amount can be administered in one or more administrations. In the case of cancer, the effective amount of the drug or composition may: (i) reduce the number of cancerous cells; (ii) reduce tumor size; (iii) inhibit, retard, slow to some extent and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “simultaneous administration” or equivalents thereof, as used herein, means that a first therapy and second therapy in a combination therapy are administered with a time separation of no more than about 15 minutes, such as no more than about any of 10 minutes, 5 minutes, or 1 minute. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

The term “sequential administration” or equivalents thereof, as used herein, means that the first therapy and second therapy in a combination therapy are administered with a time separation of more than about 15 minutes, such as more than about any of 20 minutes, 30 minutes, 40 minutes, 50 minutes, or 60 minutes. The methods disclosed herein encompass scenarios wherein either a first therapy or a second therapy may be administered first. The first and second therapies generally will be contained in separate compositions, which may be contained in the same or different packages or kits.

The term “concurrent administration” or equivalents thereof, as used herein, means that the administration of a first therapy and the administration of a second therapy in a combination therapy overlap with one another.

“Adjuvant setting” refers to a clinical setting in which an individual has had a history of cancer, and generally (but not necessarily) has been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

As used herein, an “at risk” individual is an individual who is at risk of developing cancer. An individual “at risk” may or may not have detectable disease, and may or may not have displayed detectable disease prior to administration of the treatment methods described herein. “At risk” denotes that an individual has one or more so-called risk factors, which are measurable parameters that correlate with development of a cancer, such as those described herein. An individual having one or more of these risk factors may have a higher probability of developing cancer than an individual without these risk factor(s).

The term “individual” refers to a mammal and includes, but is not limited to, human, bovine, horse, feline, canine, rodent, or primate.

“Neoadjuvant setting” refers to a clinical setting in which the method is carried out before the primary/definitive therapy.

As used herein, “delaying” the development of cancer means to defer, hinder, slow, retard, stabilize, and/or postpone development of the disease. This delay can be of varying lengths of time, depending on the history of the disease and/or individual being treated. As is evident to one of ordinary skill in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT Scan), Magnetic Resonance Imaging (MM), abdominal ultrasound, clotting tests, arteriography, or biopsy. Development may also refer to cancer progression that may be initially undetectable and includes occurrence, recurrence, and onset.

The term “pharmaceutically acceptable” or “pharmacologically compatible,” as used herein, is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers, excipients, or salts have preferably met the required standards of toxicological and manufacturing testing and/or are included on the Inactive Ingredient Guide prepared by the U.S. Food and Drug administration.

The term “based on” or “basis for,” as used herein, includes assessing, determining, obtaining, or measuring one or more characteristic of an individual or a cancer therein as described herein, and in some embodiments, selecting the individual suitable for receiving a treatment as described in the methods disclosed herein. For example, when a HR deficiency status of a cancer is used as a basis for selecting an individual for a treatment method herein, assessing (or aiding in assessing), measuring, obtaining, or determining the HR deficiency status may be included in a method of a treatment as described herein, e.g., the HR deficiency status is measured before and/or during and/or after treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### Methods for Treating a Cancer

The present application provides, in some embodiments, methods for treating a cancer in an individual in need thereof, the methods comprising administering to the individual an effective amount of a hypoxia targeting composition (such as a hypoxia-activated drug or a prodrug thereof), wherein a status of the cancer is used as a basis for selecting the individual for treatment, and the status of the cancer is one or more of: a HR deficiency status, an IDH mutation status, and a hypoxia status.

The methods described herein, in some embodiments, comprise use of a HR deficiency status of a cancer as a basis for selecting an individual for a treatment. In some embodiments, the present application provides methods for treating a cancer in an individual having an HR deficiency in the cancer or a portion thereof. In some embodiments, the method for treating a cancer in an individual comprises selecting the individual for treatment based on a positive status indicative of HR deficiency in the cancer or a portion thereof. In some embodiments, the present application provides methods of selecting (including identifying) an individual having a cancer suitable for treatment with the methods disclosed herein, wherein the method comprises determining a HR deficiency status of the cancer in the individual. In some embodiments, the present application provides methods of selecting (including identifying) an individual having a cancer suitable for treatment with the methods disclosed herein, wherein the method comprises determining a HR deficiency status of the cancer in the individual, and wherein the individual is selected if the individual has a positive status indicative of HR deficiency in the cancer or a portion thereof. In some embodiments, the HR deficiency status of the cancer is based on a HR deficiency signature. In some embodiments, the HR deficiency status of a cancer is based on one or more of the following: (i) a sequence of a gene or a product thereof; (ii) telomeric allelic imbalance (TAI); (iii) large-scale state transitions (LST); (iv) loss of heterozygosity (LOH); and (v) promoter methylation (or lack thereof). In some embodiments, the HR deficiency status of a cancer is determined based on DNA sequencing of one or more genes, or a portion thereof. In some embodiments, the HR deficiency status of a cancer is determined based on RNA sequencing of one or more genes transcripts, e.g., mRNA, or a portion thereof. In some embodiments, the HR deficiency status of a cancer is determined based on protein sequencing of one or more gene products, or a portion thereof. In some embodiments, the HR deficiency status of a cancer is determined based on one or more of the following: (i) assessing a gene sequence or a product thereof; (ii) assessing loss of heterozygosity (LOH); (iii) assessing telomeric allelic imbalance (TAI); (iv) assessing large-scale state transitions (LST); and (v) assessing promoter methylation (or lack thereof). In some embodiments, the HR deficiency status of the cancer is determined prior to administration of an effective amount of a hypoxia targeting composition (such as a hypoxia-activated drug or a prodrug thereof). In some embodiments, the methods disclosed herein further comprise determining a HR deficiency status of a cancer prior to administration of an effective amount of a hypoxia targeting composition (such as a hypoxia-activated drug or a prodrug thereof). In some embodiments, the HR deficiency status of a cancer in an individual is determined, and if the HR deficiency status is indicative of HR deficiency, the individual is administered an effective amount of a hypoxia targeting composition (such as a hypoxia-activated drug or a prodrug thereof). In some embodiments, the methods disclosed herein further comprise selecting an individual for treatment based on a HR deficiency status of a cancer. In some embodiments, the IDH mutation status of a cancer is further used as a basis for selecting the individual for treatment. In some embodiments, the hypoxia status of a cancer is further used as a basis for selecting the individual for treatment.

The methods described herein, in some embodiments, comprise use of an IDH mutation status of a cancer as a basis for selecting an individual for a treatment. In some embodiments, the present application provides methods for treating a cancer in an individual having an IDH mutation in the cancer or a portion thereof. In some embodiments, the method for treating a cancer in an individual comprises selecting the individual for treatment based on an IDH mutation status, wherein the IDH mutation status is indicative of the cancer comprising a mutation in IDH. In some embodiments, the present application provides methods of selecting (including identifying) an individual having a cancer suitable for treatment with the methods disclosed herein, wherein the method comprises determining an IDH mutation status of the cancer in the individual. In some embodiments, the present application provides methods of selecting (including identifying) an individual having a cancer suitable for treatment with the methods disclosed herein, wherein the method comprises determining an IDH mutation status of the cancer in the individual, and wherein the individual is selected if the IDH mutation status is indicative of the cancer comprising a mutation in IDH. In some embodiments, the IDH mutation status of the cancer is based on one or more of the following: (i) a gene sequence of an IDH isozyme; (ii) a change in activity of an IDH isozyme; and (iii) a level of a metabolic biomarker. In some embodiments, the IDH mutation status is based on an IDH mutation, such as one or more of an IDH1 mutation, IDH2 mutation, or IDH3 mutation. In some embodiments, the IDH mutation status of a cancer is determined based on DNA sequencing of one or more genes, or a portion thereof. In some embodiments, the IDH mutation status of a cancer is determined based on RNA sequencing of one or more genes transcripts, e.g., mRNA, or a portion thereof. In some embodiments, the IDH mutation status of a cancer is determined based on protein sequencing of one or more gene products, or a portion thereof. In some embodiments, the IDH mutation status of a cancer is determined based on one or more of the following: (i) assessing gene sequence, or product thereof, of an IDH isozyme; (ii) assessing a change in activity of an IDH isozyme; and (iii) assessing a level of a metabolic biomarker. In some embodiments, the IDH mutation status of a cancer is determined prior to administration of an effective amount of a hypoxia targeting composition (such as a hypoxia-activated drug or a prodrug thereof). In some embodiments, the methods disclosed herein further comprise determining an IDH mutation status of a cancer prior to administration of an effective amount of a hypoxia targeting composition (such as a hypoxia-activated drug or a prodrug thereof). In some embodiments, the methods disclosed herein further comprise selecting an individual for treatment based on an IDH mutation status of the cancer. In some embodiments, the IDH mutation status of a cancer in an individual is determined, and if the IDH mutation status is indicative of the cancer having an IDH mutation, the individual is administered an effective amount of a hypoxia targeting composition (such as a hypoxia-activated drug or a prodrug thereof). In some embodiments, the HR deficiency status of a cancer is further used as a basis for selecting the individual for treatment. In some embodiments, the hypoxia status of a cancer is further used as a basis for selecting the individual for treatment.

The methods described herein, in some embodiments, comprise use of a hypoxia status of a cancer as a basis for selecting an individual for a treatment. In some embodiments, the present application provides methods for treating a cancer in an individual having hypoxia in the cancer or a portion thereof. In some embodiments, the method for treating a cancer in an individual comprises selecting the individual for treatment based on a hypoxia status indicative of the cancer or a portion thereof being hypoxic. In some embodiments, the present application provides methods of selecting (including identifying) an individual having a cancer suitable for treatment with the methods disclosed herein, wherein the method comprises determining a hypoxia status of the cancer in the individual. In some embodiments, the present application provides methods of selecting (including identifying) an individual having a cancer suitable for treatment with the methods disclosed herein, wherein the method comprises determining a hypoxia status of the cancer in the individual, and wherein the individual is selected if the individual has a hypoxia status indicative of the cancer or a portion thereof being hypoxic. In some embodiments, the hypoxia status of a cancer is based on one or more of the following: (i) a tissue oxygenation level; and (ii) a hypoxia biomarker. In some embodiments, the hypoxia status of a cancer is based on a low tissue oxygenation level. In some embodiments, the low tissue oxygenation level is a tissue oxygenation level of about 4% or less of oxygen, such as about 3% or less of oxygen, about 2% or less of oxygen, or about 1% or less of oxygen. In some embodiments, the tissue oxygenation level is based on an oxygenation level obtained via an oxymetric technique. In some embodiments, the hypoxia status of the cancer is determined based on one or more of the following: (i) assessing a tissue oxygenation level, such as via an oxymetric technique; and (ii) assessing a hypoxia biomarker. In some embodiments, the hypoxia status of a cancer is determined prior to administration of an effective amount of a hypoxia targeting composition (such as a hypoxia-activated drug or a prodrug thereof). In some embodiments, the method further comprises selecting the individual for treatment based on the hypoxia status of the cancer. In some embodiments, the hypoxia status of a cancer in an individual is determined, and if the hypoxia status is indicative of the cancer or a portion thereof being hypoxic, the individual is administered an effective amount of a hypoxia targeting composition (such as a hypoxia-activated drug or a prodrug thereof). In some embodiments, the HR deficiency status of a cancer is further used as a basis for selecting the individual for treatment. In some embodiments, the IDH mutation status of a cancer is further used as a basis for selecting the individual for treatment.

In some embodiments, the method comprises administering to the individual an effective amount of a hypoxia targeting composition (such as a hypoxia-activated drug or a prodrug thereof), wherein a HR deficiency status and an IDH mutation status are used as bases for selecting the individual for treatment. In some embodiments, the method comprises administering to the individual an effective amount of a hypoxia targeting composition (such as a hypoxia-activated drug or a prodrug thereof), wherein a HR deficiency status and a hypoxia status are used as bases for selecting the individual for treatment. In some embodiments, the method comprises administering to the individual an effective amount of a hypoxia targeting composition (such as a hypoxia-activated drug or a prodrug thereof), wherein an IDH mutation status and a hypoxia status are used as bases for selecting the individual for treatment. In some embodiments, the method comprises administering to the individual an effective amount of a hypoxia targeting composition (such as a hypoxia-activated drug or a prodrug thereof), wherein a HD deficiency status, an IDH mutation status, and a hypoxia status are used as bases for selecting the individual for treatment.

In some embodiments, the method comprises administering to the individual an effective amount of tirapazamine, wherein a HR deficiency status of the cancer is used as a basis for selecting the individual for treatment. In some embodiments, the effective amount of tirapazamine is about 0.1 mg to 1000 mg, such as about 1 mg to about 500 mg, about 20 mg to about 400 mg, or about 100 mg to about 400 mg. In some embodiments, the effective amount of tirapazamine is suitable for oral administration. In some embodiments, the HR deficiency status of the cancer is determined prior to administration of an effective amount of tirapazamine. In some embodiments, the methods disclosed herein further comprise determining a HR deficiency status of a cancer prior to administration of an effective amount of tirapazamine. In some embodiments, the methods disclosed herein further comprise selecting an individual for treatment based on a HR deficiency status of a cancer. In some embodiments, the IDH mutation status of a cancer is further used as a basis for selecting the individual for treatment. In some embodiments, the hypoxia status of a cancer is further used as a basis for selecting the individual for treatment.

In some embodiments, the method comprises administering to the individual an effective amount of tirapazamine, wherein an IDH mutation status of the cancer is used as a basis for selecting the individual for treatment. In some embodiments, the effective amount of tirapazamine is about 0.1 mg to 1000 mg, such as about 1 mg to about 500 mg, about 20 mg to about 400 mg, or about 100 mg to about 400 mg. In some embodiments, the effective amount of tirapazamine is suitable for oral administration. In some embodiments, the IDH mutation status of a cancer is determined prior to administration of an effective amount of tirapazamine. In some embodiments, the methods disclosed herein further comprise determining an IDH mutation status of a cancer prior to administration of an effective amount of tirapazamine. In some embodiments, the methods disclosed herein further comprise selecting an individual for treatment based on an IDH mutation status of the cancer. In some embodiments, the HR deficiency status of a cancer is further used as a basis for selecting the individual for treatment. In some embodiments, the hypoxia status of a cancer is further used as a basis for selecting the individual for treatment.

In some embodiments, the method comprises administering to the individual an effective amount of tirapazamine, wherein a hypoxia status of the cancer is used as a basis for selecting the individual for treatment. In some embodiments, the effective amount of tirapazamine is about 0.1 mg to 1000 mg, such as about 1 mg to about 500 mg, about 20 mg to about 400 mg, or about 100 mg to about 400 mg. In some embodiments, the effective amount of tirapazamine is suitable for oral administration. In some embodiments, the hypoxia status of a cancer is determined prior to administration of an effective amount of tirapazamine. In some embodiments, the method further comprises selecting the individual for treatment based on the hypoxia status of the cancer. In some embodiments, the HR deficiency status of a cancer is further used as a basis for selecting the individual for treatment. In some embodiments, the IDH mutation status of a cancer is further used as a basis for selecting the individual for treatment.

In some embodiments, the methods disclosed herein further comprise administering to an individual another agent, including, but not limited to, a PARP inhibitor. In some embodiments, the other agent is an immune checkpoint inhibitor. In some embodiments, the immune checkpoint inhibitor is an agent that targets PD-1, PD-L1, or CTLA-4, or a ligand thereto. In some embodiments, the immune checkpoint inhibitor is selected from the group consisting of pembrolizumab, nivolumab, cemiplimab, atezolizumab, avelumab, durvalumab, and ipilimumab.

### Combination Treatments

The present application provides, in some embodiments, methods for treating a cancer in an individual in need thereof comprising combination treatments comprising: (i) an effective amount of a hypoxia targeting composition (such as a hypoxia-activated drug or a prodrug thereof), and (ii) an effective amount of a poly(ADP-ribose) polymerase (PARP) inhibitor. In some embodiments, the hypoxia targeting composition is a hypoxia-activated drug or a prodrug thereof. In some embodiments, the hypoxia targeting composition is a hypoxia-activated drug or a prodrug thereof. In some embodiments, the hypoxia targeting composition is a hypoxia-activated drug or a prodrug thereof. In some embodiments, the hypoxia-activated drug or the prodrug thereof is selected from the group consisting of: apaziquone (E09), AQ4N, etanidazole, evofosfamide (TH-302), mitomycin C, nimorazole, pimonidazole, porfiromycin, PR-104, SN30000, tarloxotinib, or tirapazamine, or an analog or derivative thereof. In some embodiments, the hypoxia-activated drug or the prodrug thereof is tirapazamine or an analog or derivative thereof. In some embodiments, the hypoxia-activated drug or the prodrug thereof is a hypoxia-activated drug. In some embodiments, the hypoxia-activated drug or the prodrug thereof is a hypoxia-activated prodrug. In some embodiments, the hypoxia-activated drug or the prodrug thereof is tirapazamine. In some embodiments, the hypoxia-activated drug or the prodrug thereof is a prodrug of tirapazamine. In some embodiments, the hypoxia-activated drug or the prodrug thereof is a derivative of tirapazamine. In some embodiments, the hypoxia-activated drug or the prodrug thereof is an analog of tirapazamine. In some embodiments, the effective amount of a hypoxia-activated drug or a prodrug thereof is about 0.1 mg to 1000 mg, such as about 1 mg to about 500 mg, about 20 mg to about 400 mg, or about 100 mg to about 400 mg. In some embodiments, the effective amount of a hypoxia-activated drug or a prodrug thereof is suitable for oral administration. In some embodiments, the PARP inhibitor is selected from the group consisting of: 3-aminobenzamine, BGD-290, CEP 9722, E7016, iniparib, niraparib, olaparib, rucaparib, talazoparib, Fluzoparib, and veliparib. In some embodiments, the PARP inhibitor is talazoparib. In some embodiments, the PARP inhibitor is olaparib. In some embodiments, the effective amount of a PARP inhibitor is about 20 mg to about 2000 mg, such as about 100 mg to about 1000 mg, about 300 mg to about 600 mg, or about 300 mg to about 1500 mg. In some embodiments, the individual is not responsive to an effective amount of a PARP inhibitor when administered alone. In some embodiments, the individual is resistant or refractory to an effective amount of a PARP inhibitor when administered alone. In some embodiments, the combination comprising: (i) an effective amount of a hypoxia targeting composition (such as a hypoxia-activated drug or a prodrug thereof), and (ii) an effective amount of a PARP inhibitor, are administered simultaneously. In some embodiments, the combination comprising: (i) an effective amount of a hypoxia targeting composition (such as a hypoxia-activated drug or a prodrug thereof), and (ii) an effective amount of a PARP inhibitor, are administered sequentially. In some embodiments, the combination comprising: (i) an effective amount of a hypoxia targeting composition (such as a hypoxia-activated drug or a prodrug thereof), and (ii) an effective amount of a PARP inhibitor, are administered concurrently.

In some embodiments, one or more of a HR deficiency status, an IDH mutation status, and a hypoxia status of a cancer is used as a basis for selecting an individual for a method comprising a combination treatment disclosed herein.

The combination methods described herein, in some embodiments, comprise use of a HR deficiency status of a cancer as a basis for selecting an individual for a treatment. In some embodiments, the present application provides methods for treating a cancer in an individual having an HR deficiency in the cancer or a portion thereof. In some embodiments, the method for treating a cancer in an individual comprises selecting the individual for treatment based on a positive status indicative of HR deficiency in the cancer or a portion thereof. In some embodiments, the present application provides methods of selecting (including identifying) an individual having a cancer suitable for treatment with the methods disclosed herein, wherein the method comprises determining a HR deficiency status of the cancer in the individual. In some embodiments, the present application provides methods of selecting (including identifying) an individual having a cancer suitable for treatment with the methods disclosed herein, wherein the method comprises determining a HR deficiency status of the cancer in the individual, and wherein the individual is selected if the individual has a positive status indicative of HR deficiency in the cancer or a portion thereof. In some embodiments, the HR deficiency status of the cancer is based on a HR deficiency signature. In some embodiments, the HR deficiency status of a cancer is based on one or more of the following: (i) a sequence of a gene or a product thereof; (ii) telomeric allelic imbalance (TAI); (iii) large-scale state transitions (LST); (iv) loss of heterozygosity (LOH); and (v) promoter methylation (or lack thereof). In some embodiments, the HR deficiency status of a cancer is determined based on DNA sequencing of one or more genes, or a portion thereof. In some embodiments, the HR deficiency status of a cancer is determined based on RNA sequencing of one or more genes transcripts, e.g., mRNA, or a portion thereof. In some embodiments, the HR deficiency status of a cancer is determined based on protein sequencing of one or more gene products, or a portion thereof. In some embodiments, the HR deficiency status of a cancer is determined based on one or more of the following: (i) assessing a gene sequence or a product thereof; (ii) assessing loss of heterozygosity (LOH); (iii) assessing telomeric allelic imbalance (TAI); (iv) assessing large-scale state transitions (LST); and (v) assessing promoter methylation (or lack thereof). In some embodiments, the HR deficiency status of the cancer is determined prior to administration of an effective amount of a hypoxia targeting composition (such as a hypoxia-activated drug or a prodrug thereof) and/or an effective amount of a PARP inhibitor. In some embodiments, the methods disclosed herein further comprise determining a HR deficiency status of a cancer prior to administration of an effective amount of a hypoxia targeting composition (such as a hypoxia-activated drug or a prodrug thereof) and/or an effective amount of a PARP inhibitor. In some embodiments, the methods disclosed herein further comprise selecting an individual for treatment based on a HR deficiency status of a cancer. In some embodiments, the HR deficiency status of a cancer in an individual is determined, and if the HR deficiency status is indicative of HR deficiency, the individual is administered (i) an effective amount of a hypoxia targeting composition (such as a hypoxia-activated drug or a prodrug thereof); and (ii) an effective amount of a PARP inhibitor. In some embodiments, the IDH mutation status of a cancer is further used as a basis for selecting the individual for treatment. In some embodiments, the hypoxia status of a cancer is further used as a basis for selecting the individual for treatment.

The combination methods described herein, in some embodiments, comprise use of an IDH mutation status of a cancer as a basis for selecting an individual for a treatment. In some embodiments, the present application provides methods for treating a cancer in an individual having an IDH mutation in the cancer or a portion thereof. In some embodiments, the method for treating a cancer in an individual comprises selecting the individual for treatment based on an IDH mutation status, wherein the IDH mutation status is indicative of the cancer comprising a mutation in IDH. In some embodiments, the present application provides methods of selecting (including identifying) an individual having a cancer suitable for treatment with the methods disclosed herein, wherein the method comprises determining an IDH mutation status of the cancer in the individual. In some embodiments, the present application provides methods of selecting (including identifying) an individual having a cancer suitable for treatment with the methods disclosed herein, wherein the method comprises determining an IDH mutation status of the cancer in the individual, and wherein the individual is selected if the IDH mutation status is indicative of the cancer comprising a mutation in IDH. In some embodiments, the IDH mutation status of the cancer is based on one or more of the following: (i) a gene sequence of an IDH isozyme; (ii) a change in activity of an IDH isozyme; and (iii) a level of a metabolic biomarker. In some embodiments, the IDH mutation status is based on an IDH mutation, such as one or more of an IDH1 mutation, IDH2 mutation, or IDH3 mutation. In some embodiments, the IDH mutation status of a cancer is determined based on DNA sequencing of one or more genes, or a portion thereof. In some embodiments, the IDH mutation status of a cancer is determined based on RNA sequencing of one or more genes transcripts, e.g., mRNA, or a portion thereof. In some embodiments, the IDH mutation status of a cancer is determined based on protein sequencing of one or more gene products, or a portion thereof. In some embodiments, the IDH mutation status of a cancer is determined based on one or more of the following: (i) assessing gene sequence, or product thereof, of an IDH isozyme; (ii) assessing a change in activity of an IDH isozyme; and (iii) assessing a level of a metabolic biomarker. In some embodiments, the IDH mutation status of a cancer is determined prior to administration of an effective amount of a hypoxia targeting composition (such as a hypoxia-activated drug or a prodrug thereof) and/or an effective amount of a PARP inhibitor. In some embodiments, the methods disclosed herein further comprise determining an IDH mutation status of a cancer prior to administration of an effective amount of a hypoxia targeting composition (such as a hypoxia-activated drug or a prodrug thereof) and/or an effective amount of a PARP inhibitor. In some embodiments, the methods disclosed herein further comprise selecting an individual for treatment based on an IDH mutation status of the cancer. In some embodiments, the IDH mutation status of a cancer in an individual is determined, and if the IDH mutation status is indicative of the cancer having an IDH mutation, the individual is administered (i) an effective amount of a hypoxia targeting composition (such as a hypoxia-activated drug or a prodrug thereof); and (ii) an effective amount of a PARP inhibitor. In some embodiments, the HR deficiency status of a cancer is further used as a basis for selecting the individual for treatment. In some embodiments, the hypoxia status of a cancer is further used as a basis for selecting the individual for treatment.

The combination methods described herein, in some embodiments, comprise use of a hypoxia status of a cancer as a basis for selecting an individual for a treatment. In some embodiments, the present application provides methods for treating a cancer in an individual having hypoxia in the cancer or a portion thereof. In some embodiments, the method for treating a cancer in an individual comprises selecting the individual for treatment based on a hypoxia status indicative of the cancer or a portion thereof being hypoxic. In some embodiments, the present application provides methods of selecting (including identifying) an individual having a cancer suitable for treatment with the methods disclosed herein, wherein the method comprises determining a hypoxia status of the cancer in the individual. In some embodiments, the present application provides methods of selecting (including identifying) an individual having a cancer suitable for treatment with the methods disclosed herein, wherein the method comprises determining a hypoxia status of the cancer in the individual, and wherein the individual is selected if the individual has a hypoxia status indicative of the cancer or a portion thereof being hypoxic. In some embodiments, the hypoxia status of a cancer is based on one or more of the following: (i) a tissue oxygenation level; and (ii) a hypoxia biomarker. In some embodiments, the hypoxia status of a cancer is based on a low tissue oxygenation level. In some embodiments, the low tissue oxygenation level is a tissue oxygenation level of about 4% or less of oxygen, such as about 3% or less of oxygen, about 2% or less of oxygen, or about 1% or less of oxygen. In some embodiments, the tissue oxygenation level is based on an oxygenation level obtained via an oxymetric technique. In some embodiments, the hypoxia status of the cancer is determined based on one or more of the following: (i) assessing a tissue oxygenation level, such as via an oxymetric technique; and (ii) assessing a hypoxia biomarker. In some embodiments, the hypoxia status of the cancer is determined prior to administration of an effective amount of a hypoxia targeting composition (such as a hypoxia-activated drug or a prodrug thereof) and/or an effective amount of a PARP inhibitor. In some embodiments, the methods disclosed herein further comprise determining a hypoxia status of a cancer prior to administration of an effective amount of a hypoxia targeting composition (such as a hypoxia-activated drug or a prodrug thereof) and/or an effective amount of a PARP inhibitor. In some embodiments, the method further comprises selecting the individual for treatment based on the hypoxia status of the cancer. In some embodiments, the hypoxia status of a cancer in an individual is determined, and if the hypoxia status is indicative of the cancer or a portion thereof being hypoxic, the individual is administered (i) an effective amount of a hypoxia targeting composition (such as a hypoxia-activated drug or a prodrug thereof); and (ii) an effective amount of a PARP inhibitor. In some embodiments, the HR deficiency status of a cancer is further used as a basis for selecting the individual for treatment. In some embodiments, the IDH mutation status of a cancer is further used as a basis for selecting the individual for treatment.

In some embodiments, the method comprises administering to the individual: (i) an effective amount of a hypoxia targeting composition (such as a hypoxia-activated drug or a prodrug thereof); and (ii) an effective amount of a PARP inhibitor, wherein a HR deficiency status and an IDH mutation status are used as bases for selecting the individual for treatment. In some embodiments, the method comprises administering to the individual: (i) an effective amount of a hypoxia targeting composition (such as a hypoxia-activated drug or a prodrug thereof); and (ii) an effective amount of a PARP inhibitor, wherein a HR deficiency status and a hypoxia status are used as bases for selecting the individual for treatment. In some embodiments, the method comprises administering to the individual: (i) an effective amount of a hypoxia targeting composition (such as a hypoxia-activated drug or a prodrug thereof); and (ii) an effective amount of a PARP inhibitor, wherein an IDH mutation status and a hypoxia status are used as bases for selecting the individual for treatment. In some embodiments, the method comprises administering to the individual: (i) an effective amount of a hypoxia targeting composition (such as a hypoxia-activated drug or a prodrug thereof); and (ii) an effective amount of a PARP inhibitor, wherein a HD deficiency status, an IDH mutation status, and a hypoxia status are used as bases for selecting the individual for treatment.

In some embodiments, the method comprises administering to the individual: (i) an effective amount of a hypoxia targeting composition (such as a hypoxia-activated drug or a prodrug thereof), and (ii) an effective amount of a poly(ADP-ribose) polymerase (PARP) inhibitor wherein a HR deficiency status of a cancer is used as a basis for selecting the individual for treatment. In some embodiments, the hypoxia targeting composition is a hypoxia-activated drug or a prodrug thereof. In some embodiments, the hypoxia-activated drug or the prodrug thereof is selected from the group consisting of: apaziquone (E09), AQ4N, etanidazole, evofosfamide (TH-302), mitomycin C, nimorazole, pimonidazole, porfiromycin, PR-104, SN30000, tarloxotinib, or tirapazamine, or an analog or derivative thereof. In some embodiments, the hypoxia-activated drug or the prodrug thereof is a hypoxia-activated drug. In some embodiments, the hypoxia-activated drug or the prodrug thereof is a hypoxia-activated prodrug. In some embodiments, the hypoxia-activated drug or the prodrug thereof is tirapazamine or an analog or derivative thereof. In some embodiments, the hypoxia-activated drug or the prodrug thereof is tirapazamine. In some embodiments, the hypoxia-activated drug or the prodrug thereof is a prodrug of tirapazamine. In some embodiments, the hypoxia-activated drug or the prodrug thereof is a derivative of tirapazamine. In some embodiments, the hypoxia-activated drug or the prodrug thereof is an analog of tirapazamine. In some embodiments, the effective amount of the hypoxia targeting composition (such as the hypoxia-activated drug or the prodrug thereof) is about 0.1 mg to 1000 mg, such as about 1 mg to about 500 mg, about 20 mg to about 400 mg, or about 100 mg to about 400 mg. In some embodiments, the effective amount of the hypoxia targeting composition (such as the hypoxia-activated drug or the prodrug thereof) is suitable for oral administration. In some embodiments, the PARP inhibitor is selected from the group consisting of: 3-aminobenzamine, BGD-290, CEP 9722, E7016, iniparib, niraparib, olaparib, rucaparib, talazoparib, Fluzoparib, and veliparib. In some embodiments, the PARP inhibitor is talazoparib. In some embodiments, the PARP inhibitor is olaparib. In some embodiments, the effective amount of the PARP inhibitor is about 20 mg to about 2000 mg, such as about 100 mg to about 1000 mg, about 300 mg to about 600 mg, or about 300 mg to about 1500 mg. In some embodiments, the individual is not responsive to an effective amount of a PARP inhibitor when administered alone. In some embodiments, the individual is resistant or refractory to an effective amount of a PARP inhibitor when administered alone. In some embodiments, the combination comprising: (i) the effective amount of the hypoxia targeting composition (such as the hypoxia-activated drug or the prodrug thereof), and (ii) the effective amount of the PARP inhibitor, are administered simultaneously. In some embodiments, the combination comprising: (i) the effective amount of the hypoxia targeting composition (such as the hypoxia-activated drug or the prodrug thereof), and (ii) the effective amount of the PARP inhibitor, are administered sequentially. In some embodiments, the combination comprising: (i) the effective amount of the hypoxia targeting composition (such as the hypoxia-activated drug or the prodrug thereof), and (ii) the effective amount of the PARP inhibitor, are administered concurrently. In some embodiments, the HR deficiency status of the cancer is based on a HR deficiency signature. In some embodiments, the HR deficiency status of a cancer is based on one or more of the following: (i) a sequence of a gene or a product thereof; (ii) telomeric allelic imbalance (TAI); (iii) large-scale state transitions (LST); (iv) loss of heterozygosity (LOH); and (v) promoter methylation (or lack thereof). In some embodiments, the HR deficiency status of a cancer is determined based on DNA sequencing of one or more genes, or a portion thereof. In some embodiments, the HR deficiency status of a cancer is determined based on RNA sequencing of one or more genes transcripts, e.g., mRNA, or a portion thereof. In some embodiments, the HR deficiency status of a cancer is determined based on protein sequencing of one or more gene products, or a portion thereof. In some embodiments, the HR deficiency status of a cancer is determined based on one or more of the following: (i) assessing loss of heterozygosity (LOH); (ii) assessing telomeric allelic imbalance (TAI); (iii) assessing large-scale state transitions (LST); (iv) assessing a gene sequence or a product thereof and (v) assessing promoter methylation (or lack thereof). In some embodiments, the HR deficiency status of the cancer is determined prior to administration of an effective amount of a hypoxia targeting composition (such as a hypoxia-activated drug or a prodrug thereof). In some embodiments, the methods disclosed herein further comprise determining a HR deficiency status of a cancer prior to administration of an effective amount of a hypoxia targeting composition (such as a hypoxia-activated drug or a prodrug thereof). In some embodiments, the methods disclosed herein further comprise selecting an individual for treatment based on a HR deficiency status of a cancer. In some embodiments, the IDH mutation status of a cancer is further used as a basis for selecting the individual for treatment. In some embodiments, the hypoxia status of a cancer is further used as a basis for selecting the individual for treatment.

In some embodiments, the method comprises administering to the individual: (i) an effective amount of a hypoxia targeting composition (such as a hypoxia-activated drug or a prodrug thereof), and (ii) an effective amount of a poly(ADP-ribose) polymerase (PARP) inhibitor, wherein an IDH mutation status of the cancer is used as a basis for selecting the individual for treatment. In some embodiments, the hypoxia targeting composition is a hypoxia-activated drug or a prodrug thereof. In some embodiments, the hypoxia-activated drug or the prodrug thereof is selected from the group consisting of: apaziquone (E09), AQ4N, etanidazole, evofosfamide (TH-302), mitomycin C, nimorazole, pimonidazole, porfiromycin, PR-104, SN30000, tarloxotinib, or tirapazamine, or an analog or derivative thereof. In some embodiments, the hypoxia-activated drug or the prodrug thereof is a hypoxia-activated drug. In some embodiments, the hypoxia-activated drug or the prodrug thereof is a hypoxia-activated prodrug. In some embodiments, the hypoxia-activated drug or the prodrug thereof is tirapazamine or an analog or derivative thereof. In some embodiments, the hypoxia-activated drug or the prodrug thereof is tirapazamine. In some embodiments, the hypoxia-activated drug or the prodrug thereof is a prodrug of tirapazamine. In some embodiments, the hypoxia-activated drug or the prodrug thereof is a derivative of tirapazamine. In some embodiments, the hypoxia-activated drug or the prodrug thereof is an analog of tirapazamine. In some embodiments, the effective amount of the hypoxia targeting composition (such as the hypoxia-activated drug or the prodrug thereof) is about 0.1 mg to 1000 mg, such as about 1 mg to about 500 mg, about 20 mg to about 400 mg, or about 100 mg to about 400 mg. In some embodiments, the effective amount of the hypoxia targeting composition (such as the hypoxia-activated drug or the prodrug thereof) is suitable for oral administration. In some embodiments, the PARP inhibitor is selected from the group consisting of: 3-aminobenzamine, BGD-290, CEP 9722, E7016, iniparib, niraparib, olaparib, rucaparib, talazoparib, Fluzoparib, and veliparib. In some embodiments, the PARP inhibitor is talazoparib. In some embodiments, the PARP inhibitor is olaparib. In some embodiments, the effective amount of the PARP inhibitor is about 20 mg to about 2000 mg, such as about 100 mg to about 1000 mg, about 300 mg to about 600 mg, or about 300 mg to about 1500 mg. In some embodiments, the individual is not responsive to an effective amount of a PARP inhibitor when administered alone. In some embodiments, the individual is resistant or refractory to an effective amount of a PARP inhibitor when administered alone. In some embodiments, the combination comprising: (i) the effective amount of the hypoxia targeting composition (such as the hypoxia-activated drug or the prodrug thereof), and (ii) the effective amount of the PARP inhibitor, are administered simultaneously. In some embodiments, the combination comprising: (i) the effective amount of the hypoxia targeting composition (such as the hypoxia-activated drug or the prodrug thereof), and (ii) the effective amount of the PARP inhibitor, are administered sequentially. In some embodiments, the combination comprising: (i) the effective amount of the hypoxia targeting composition (such as the hypoxia-activated drug or the prodrug thereof), and (ii) the effective amount of the PARP inhibitor, are administered concurrently. In some embodiments, the IDH mutation status of the cancer is based on one or more of the following: (i) a gene sequence of an IDH isozyme; (ii) a change in activity of an IDH isozyme; and (iii) a level of a metabolic biomarker. In some embodiments, the IDH mutation status is based on an IDH mutation, such as one or more of an IDH1 mutation, IDH2 mutation, or IDH3 mutation. In some embodiments, the IDH mutation status of a cancer is determined based on DNA sequencing of one or more genes, or a portion thereof. In some embodiments, the IDH mutation status of a cancer is determined based on RNA sequencing of one or more genes transcripts, e.g., mRNA, or a portion thereof. In some embodiments, the IDH mutation status of a cancer is determined based on protein sequencing of one or more gene products, or a portion thereof. In some embodiments, the IDH mutation status of a cancer is determined based on one or more of the following: (i) assessing gene sequence, or product thereof, of an IDH isozyme; (ii) assessing a change in activity of an IDH isozyme; and (iii) assessing a level of a metabolic biomarker. In some embodiments, the IDH mutation status of a cancer is determined prior to administration of an effective amount of a hypoxia targeting composition (such as a hypoxia-activated drug or a prodrug thereof). In some embodiments, the methods disclosed herein further comprise determining an IDH mutation status of a cancer prior to administration of an effective amount of a hypoxia targeting composition (such as a hypoxia-activated drug or a prodrug thereof). In some embodiments, the methods disclosed herein further comprise selecting an individual for treatment based on an IDH mutation status of the cancer. In some embodiments, the HR deficiency status of a cancer is further used as a basis for selecting the individual for treatment. In some embodiments, the hypoxia status of a cancer is further used as a basis for selecting the individual for treatment.

In some embodiments, the method comprises administering to the individual: (i) an effective amount of a hypoxia targeting composition (such as a hypoxia-activated drug or a prodrug thereof), and (ii) an effective amount of a poly(ADP-ribose) polymerase (PARP) inhibitor, wherein a hypoxia status of the cancer is used as a basis for selecting the individual for treatment. In some embodiments, the hypoxia targeting composition is a hypoxia-activated drug or a prodrug thereof. In some embodiments, the hypoxia-activated drug or the prodrug thereof is selected from the group consisting of: apaziquone (E09), AQ4N, etanidazole, evofosfamide (TH-302), mitomycin C, nimorazole, pimonidazole, porfiromycin, PR-104, SN30000, tarloxotinib, or tirapazamine, or an analog or derivative thereof. In some embodiments, the hypoxia-activated drug or the prodrug thereof is a hypoxia-activated drug. In some embodiments, the hypoxia-activated drug or the prodrug thereof is a hypoxia-activated prodrug. In some embodiments, the hypoxia-activated drug or the prodrug thereof is tirapazamine or an analog or derivative thereof. In some embodiments, the hypoxia-activated drug or the prodrug thereof is tirapazamine. In some embodiments, the hypoxia-activated drug or the prodrug thereof is a prodrug of tirapazamine. In some embodiments, the hypoxia-activated drug or the prodrug thereof is a derivative of tirapazamine. In some embodiments, the hypoxia-activated drug or the prodrug thereof is an analog of tirapazamine. In some embodiments, the effective amount of the hypoxia targeting composition (such as the hypoxia-activated drug or the prodrug thereof) is about 0.1 mg to 1000 mg, such as about 1 mg to about 500 mg, about 20 mg to about 400 mg, or about 100 mg to about 400 mg. In some embodiments, the effective amount of the hypoxia targeting composition (such as the hypoxia-activated drug or the prodrug thereof) is suitable for oral administration. In some embodiments, the PARP inhibitor is selected from the group consisting of: 3-aminobenzamine, BGD-290, CEP 9722, E7016, iniparib, niraparib, olaparib, rucaparib, talazoparib, and Fluzoparib, veliparib. In some embodiments, the PARP inhibitor is talazoparib. In some embodiments, the PARP inhibitor is olaparib. In some embodiments, the effective amount of the PARP inhibitor is about 20 mg to about 2000 mg, such as about 100 mg to about 1000 mg, about 300 mg to about 600 mg, or about 300 mg to about 1500 mg. In some embodiments, the individual is not responsive to an effective amount of a PARP inhibitor when administered alone. In some embodiments, the individual is resistant or refractory to an effective amount of a PARP inhibitor when administered alone. In some embodiments, the combination comprising: (i) the effective amount of the hypoxia targeting composition (such as the hypoxia-activated drug or the prodrug thereof), and (ii) the effective amount of the PARP inhibitor, are administered simultaneously. In some embodiments, the combination comprising: (i) the effective amount of the hypoxia targeting composition (such as the hypoxia-activated drug or the prodrug thereof), and (ii) the effective amount of the PARP inhibitor, are administered sequentially. In some embodiments, the combination comprising: (i) the effective amount of the hypoxia targeting composition (such as the hypoxia-activated drug or the prodrug thereof), and (ii) the effective amount of the PARP inhibitor, are administered concurrently. In some embodiments, the hypoxia status of a cancer is based on one or more of the following: (i) a tissue oxygenation level; and (ii) a hypoxia biomarker. In some embodiments, the hypoxia status of a cancer is based on a low tissue oxygenation level. In some embodiments, the low tissue oxygenation level is a tissue oxygenation level of about 4% or less of oxygen, such as about 3% or less of oxygen, about 2% or less of oxygen, or about 1% or less of oxygen. In some embodiments, the tissue oxygenation level is based on an oxygenation level obtained via an oxymetric technique. In some embodiments, the hypoxia status of the cancer is determined based on one or more of the following: (i) assessing a tissue oxygenation level, such as via an oxymetric technique; and (ii) assessing a hypoxia biomarker. In some embodiments, the hypoxia status of a cancer is determined prior to administration of an effective amount of a hypoxia targeting composition (such as a hypoxia-activated drug or a prodrug thereof). In some embodiments, the method further comprises selecting the individual for treatment based on the hypoxia status of the cancer. In some embodiments, the HR deficiency status of a cancer is further used as a basis for selecting the individual for treatment. In some embodiments, the IDH mutation status of a cancer is further used as a basis for selecting the individual for treatment.

In some embodiments, the method comprises administering to the individual: (i) an effective amount of tirapazamine, and (ii) an effective amount of a PARP inhibitor, wherein the PARP inhibitor is selected from the group consisting of olaparib, rucaparib, niraparib, and veliparib. In some embodiments, the effective amount of tirapazamine is about 0.1 mg to 1000 mg, such as about 1 mg to about 500 mg, about 20 mg to about 400 mg, or about 100 mg to about 400 mg. In some embodiments, the effective amount of tirapazamine is suitable for oral administration. In some embodiments, the effective amount of a PARP inhibitor is about 20 mg to about 2000 mg, such as about 100 mg to about 1000 mg, about 300 mg to about 600 mg, or about 300 mg to about 1500 mg. In some embodiments, the individual is not responsive to an effective amount of a PARP inhibitor when administered alone. In some embodiments, the individual is resistant or refractory to an effective amount of a PARP inhibitor when administered alone. In some embodiments, the combination comprising: (i) an effective amount of tirapazamine, and (ii) an effective amount of a PARP inhibitor, are administered simultaneously. In some embodiments, the combination comprising: (i) an effective amount of tirapazamine, and (ii) an effective amount of a PARP inhibitor, are administered sequentially. In some embodiments, the combination comprising: (i) an effective amount of tirapazamine, and (ii) an effective amount of a PARP inhibitor, are administered concurrently.

In some embodiments, the method for treating a cancer in an individual in need thereof comprises administering to the individual: (i) an effective amount of tirapazamine, and (ii) an effective amount of a poly(ADP-ribose) polymerase (PARP) inhibitor, wherein the PARP inhibitor is selected from the group consisting of olaparib, rucaparib, niraparib, and veliparib, and wherein a HR deficiency status of a cancer is used as a basis for selecting the individual for treatment. In some embodiments, the effective amount of tirapazamine is about 0.1 mg to 1000 mg, such as about 1 mg to about 500 mg, about 20 mg to about 400 mg, or about 100 mg to about 400 mg. In some embodiments, the effective amount of tirapazamine is suitable for oral administration. In some embodiments, the effective amount of the PARP inhibitor is about 20 mg to about 2000 mg, such as about 100 mg to about 1000 mg, about 300 mg to about 600 mg, or about 300 mg to about 1500 mg. In some embodiments, the individual is not responsive to an effective amount of a PARP inhibitor when administered alone. In some embodiments, the individual is resistant or refractory to an effective amount of a PARP inhibitor when administered alone. In some embodiments, the combination comprising: (i) the effective amount of tirapazamine, and (ii) the effective amount of the PARP inhibitor, are administered simultaneously. In some embodiments, the combination comprising: (i) the effective amount of tirapazamine, and (ii) the effective amount of the PARP inhibitor, are administered sequentially. In some embodiments, the combination comprising: (i) the effective amount of tirapazamine, and (ii) the effective amount of the PARP inhibitor, are administered concurrently. In some embodiments, the HR deficiency status of the cancer is determined prior to administration of an effective amount of tirapazamine. In some embodiments, the methods disclosed herein further comprise determining a HR deficiency status of a cancer prior to administration of an effective amount of tirapazamine. In some embodiments, the methods disclosed herein further comprise selecting an individual for treatment based on a HR deficiency status of a cancer. In some embodiments, the IDH mutation status of a cancer is further used as a basis for selecting the individual for treatment. In some embodiments, the hypoxia status of a cancer is further used as a basis for selecting the individual for treatment.

In some embodiments, the method comprises administering to the individual: (i) an effective amount of tirapazamine, and (ii) an effective amount of a poly(ADP-ribose) polymerase (PARP) inhibitor, wherein the PARP inhibitor is selected from the group consisting of olaparib, rucaparib, niraparib, and wherein an IDH mutation status of the cancer is used as a basis for selecting the individual for treatment. In some embodiments, the effective amount of tirapazamine is about 0.1 mg to 1000 mg, such as about 1 mg to about 500 mg, about 20 mg to about 400 mg, or about 100 mg to about 400 mg. In some embodiments, the effective amount of tirapazamine is suitable for oral administration. In some embodiments, the effective amount of the PARP inhibitor is about 20 mg to about 2000 mg, such as about 100 mg to about 1000 mg, about 300 mg to about 600 mg, or about 300 mg to about 1500 mg. In some embodiments, the individual is not responsive to an effective amount of a PARP inhibitor when administered alone. In some embodiments, the individual is resistant or refractory to an effective amount of a PARP inhibitor when administered alone. In some embodiments, the combination comprising: (i) the effective amount of tirapazamine, and (ii) the effective amount of the PARP inhibitor, are administered simultaneously. In some embodiments, the combination comprising: (i) the effective amount of tirapazamine, and (ii) the effective amount of the PARP inhibitor, are administered sequentially. In some embodiments, the combination comprising: (i) the effective amount of tirapazamine, and (ii) the effective amount of the PARP inhibitor, are administered concurrently. In some embodiments, the IDH mutation status of a cancer is determined prior to administration of an effective amount of tirapazamine. In some embodiments, the methods disclosed herein further comprise determining an IDH mutation status of a cancer prior to administration of an effective amount of tirapazamine. In some embodiments, the methods disclosed herein further comprise selecting an individual for treatment based on an IDH mutation status of the cancer. In some embodiments, the HR deficiency status of a cancer is further used as a basis for selecting the individual for treatment. In some embodiments, the hypoxia status of a cancer is further used as a basis for selecting the individual for treatment.

In some embodiments, the method comprises administering to the individual: (i) an effective amount of tirapazamine, and (ii) an effective amount of a poly(ADP-ribose) polymerase (PARP) inhibitor, wherein the PARP inhibitor is selected from the group consisting of olaparib, rucaparib, niraparib, and wherein a hypoxia status of the cancer is used as a basis for selecting the individual for treatment. In some embodiments, the effective amount of tirapazamine is about 0.1 mg to 1000 mg, such as about 1 mg to about 500 mg, about 20 mg to about 400 mg, or about 100 mg to about 400 mg. In some embodiments, the effective amount of tirapazamine is suitable for oral administration. In some embodiments, the effective amount of the PARP inhibitor is about 20 mg to about 2000 mg, such as about 100 mg to about 1000 mg, about 300 mg to about 600 mg, or about 300 mg to about 1500 mg. In some embodiments, the individual is not responsive to an effective amount of a PARP inhibitor when administered alone. In some embodiments, the individual is resistant or refractory to an effective amount of a PARP inhibitor when administered alone. In some embodiments, the combination comprising: (i) the effective amount of tirapazamine, and (ii) the effective amount of the PARP inhibitor, are administered simultaneously. In some embodiments, the combination comprising: (i) the effective amount of tirapazamine, and (ii) the effective amount of the PARP inhibitor, are administered sequentially. In some embodiments, the combination comprising: (i) the effective amount of tirapazamine, and (ii) the effective amount of the PARP inhibitor, are administered concurrently. In some embodiments, the hypoxia status of a cancer is determined prior to administration of an effective amount of tirapazamine. In some embodiments, the method further comprises selecting the individual for treatment based on the hypoxia status of the cancer. In some embodiments, the HR deficiency status of a cancer is further used as a basis for selecting the individual for treatment. In some embodiments, the IDH mutation status of a cancer is further used as a basis for selecting the individual for treatment.

In some embodiments, the combination treatment methods disclosed herein further comprise administering to an individual another agent. In some embodiments, the other agent is an immune checkpoint inhibitor. In some embodiments, the immune checkpoint inhibitor is an agent that targets PD-1, PD-L1, or CTLA-4, or a ligand thereto. In some embodiments, the immune checkpoint inhibitor is selected from the group consisting of pembrolizumab, nivolumab, cemiplimab, atezolizumab, avelumab, durvalumab, and ipilimumab.

### Homologous Recombination (HR) Status

In some embodiments, the HR deficiency status of a cancer is used as a basis for selecting an individual for any treatment method disclosed herein. Homologous recombination, via the homologous recombination repair pathway, is a cellular mechanism that, e.g., repairs double-stranded breaks and interstrand crosslinks in DNA. Homologous recombination assists, in part, with high-fidelity duplication of the genome during replication, thus reducing, e.g., erroneous DNA mutations and aberrations of oncogenes and tumor suppressor genes associate with cancer progression. See, e.g., Torgovnick, A. et al., Front Genet, 6, 2015; and Li, X. et al., Cell Res, 18, 2008, 99-113, which are hereby incorporated by reference in their entirety.

In some embodiments, the HR deficiency status of a cancer is a positive status indicative of HR deficiency in a cancer or a portion thereof. In some embodiments, the HR deficiency status of a cancer is a negative status indicative of substantially no HR deficiency, or alternatively is indicative of HR proficiency, in a cancer or a portion thereof.

In some embodiments, the HR deficiency status of a cancer is based on a HR deficiency signature, which is indicative of HR deficiency in the cancer or a portion thereof. HR deficiency signatures, such as gene sequences, gene expression levels, and epigenetic markers, are known in the art, e.g., WO2014138101 and US20170283879, which are hereby incorporated by reference in their entirety. In some embodiments, the HR deficiency status, such as based on a HR deficiency signature, of a cancer is based on one or more of the following: (i) a gene sequence, or a product thereof, or an expression level thereof; (ii) loss of heterozygosity (LOH); (iii) telomeric allelic imbalance (TAI); (iv) large-scale state transitions (LST); and (v) promoter methylation. In some embodiments, the HR deficiency signature is based on a sequence of a gene or an expression product thereof. In some embodiments, the sequence of a gene or an expression product thereof indicates a genetic mutation, as compared to a control (e.g., a gene sequence of that gene in a non-cancerous tissue or a known wild type sequence). In some embodiments, the HR deficiency signature is based on a mutation in one or more of BRCA 1, BRCA2, IDH, XRCC3, FANCD1, PALB2 or FANCN, RAD51, RAD52, FANCJ, FANCD2, DSS1, MRE11, RAD50, NBS1, BLM, ATM, ATR, CHK1, CHK2, and Fanconi anemia complementation group (FANC) A,-B,-C, -E, -F, -G,-L, M, and D2. In some embodiments, the HR deficiency signature is based on a loss-of-function mutation in one or more of BRCA 1, BRCA2, IDH, XRCC3, FANCD1, PALB2 or FANCN, RAD51, RAD52, FANCJ, FANCD2, DSS1, MRE11, RAD50, NBS1, BLM, ATM, ATR, CHK1, CHK2, and Fanconi anemia complementation group (FANC) A,-B,-C, -E, -F, -G,-L, M, and D2. In some embodiments, the HR deficiency signature is based on a reduced expression and/or product function in one or more of BRCA 1, BRCA2, IDH, XRCC3, FANCD1, PALB2 or FANCN, RAD51, RAD52, FANCJ, FANCD2, DSS1, MRE11, RAD50, NBS1, BLM, ATM, ATR, CHK1, CHK2, and Fanconi anemia complementation group (FANC) A,-B,-C, -E, -F, -G,-L, M, and D2.

In some embodiments, the HR deficiency signature is based on an expression level profile. In some embodiments, the expression level profile comprises expression level information, such as up- and/or down-regulation of a gene as compared to a control, of one or more of: FOX03, VAMP 5, CSE1L, SLC45A3, HSD1 1B2, RFC4, C6orf48, FAM43A, SERTAD4, and C4orf34. See, e.g., WO2014138101, including Table 2 disclosed therein.

In some embodiments, the HR deficiency signature is based on one or more of: loss of heterozygosity (LOH), telomeric allelic imbalance (TAI), and large-scale state transitions (LST). See, e.g., US20170283879; Birkbak, N. J. et al., Cancer Discov, 2, 2012, 366-375; Abkevich, V. et al., Br J Cancer, 107, 2012, 1776-1782; Popova, T. et al., Cancer Res, 2012, 72, 5454-5462; and Telli, M. et al., Clin Cancer res, 22, 2016, 3764-3773, which are hereby incorporated by reference in their entirety.

In some embodiments, the HR deficiency status of a cancer is based on a HR deficiency score. HR deficiency scores are known in the art, e.g., Telli, M. L., Breast Cancer Res Treat, 168, 2018, 625-630; and Sztupinszki, Z. NPJ Breast Cancer, 2018, 3, which are hereby incorporated by reference in their entirety. In some embodiments, the HR deficiency score is based on, in part or in whole, any HR deficiency assessment technique disclosed herein. In some embodiments, the HR deficiency score is indicative of HR deficiency in the cancer or a portion thereof.

In some embodiments, the HR deficiency status of a cancer is determined based on one or more of the following: (i) assessing a gene sequence, or a product thereof, or an expression level thereof; (ii) assessing loss of heterozygosity (LOH); (iii) assessing telomeric allelic imbalance (TAI); (iv) assessing large-scale state transitions (LST); and (v) assessing promoter methylation. Techniques for determining HR deficiency status are known in the art. For example, in some embodiments, the HR deficiency status is determined by sequence analysis, e.g., by sequencing analysis and/or detection of genomic DNA, or expression products therefrom (such as RNA or protein), of a sample obtained from an individual. In some embodiments, the HR deficiency status of the cancer is based on one or more of: DNA sequencing, DNA sequence detection, RNA sequencing, RNA transcript detection, protein sequencing, and protein detection. Methods for sequencing and/or detecting a gene and/or a gene expression product are well known in the art and include, but are not limited to, a high-throughput DNA sequencing method, Massively Parallel Signature Sequencing (MPSS), polony sequencing, pyrosequencing, SOLid sequencing, nanopore sequencing, immunological assays, nuclease protection assays, northern blots, in situ hybridization, ELISA, reverse transcriptase Polymerase Chain Reaction (RT-PCR), Real-Time Polymerase Chain Reaction, expressed sequence tag (EST) sequencing, cDNA microarray hybridization or gene chip analysis, subtractive cloning, Serial Analysis of Gene Expression (SAGE), Sequencing-By-Synthesis (SBS), aptamer-based assays, western blot, enzyme immunoassays, Luminex Platform utilizing color, and mass spectrometry.

In some embodiments, the methods disclosed herein further comprise assessing, such as determining, a HR deficiency status of a cancer of an individual. In some embodiments, assessing a HR deficiency status comprises determining a HR deficiency signature, such as a HR deficiency signature comprising information of a gene mutation and/or an expression level profile. In some embodiments, the HR deficiency status of a cancer is based on sequencing of DNA or a portion thereof. In some embodiments, the HR deficiency status of a cancer is based on sequencing of RNA or a portion thereof. In some embodiments, the HR deficiency status of a cancer is based on sequencing of a protein or a portion thereof.

In some embodiments, assessing a HR deficiency status comprises comparing a HR deficiency signature to a control (such as a HR deficiency signature from a non-cancerous tissue or a known HR deficiency signature indicative of HR proficiency).

In some embodiments, the HR deficiency status of a cancer is determined prior to administration of an agent disclosed in the methods described herein. For example, in some embodiments, the HR deficiency status of a cancer is determined prior to administration of an effective amount of a hypoxia targeting composition (such as a hypoxia-activated drug or a prodrug thereof). In some embodiments, the HR deficiency status of a cancer is determined prior to administration of (i) an effective amount of a hypoxia targeting composition (such as a hypoxia-activated drug or a prodrug thereof), and (ii) an effective amount of a PARP inhibitor. In some embodiments, the HR deficiency status of a cancer is further evaluated during or after a treatment method described herein.

In some embodiments, any of the methods disclosed herein further comprise selecting an individual for treatment based on a HR deficiency status of the cancer. In some embodiments, any of the methods disclosed herein further comprise selecting an individual for treatment based on a HR deficiency status of a cancer, wherein the HR deficiency status of the cancer is based on a HR deficiency signature indicative of HR deficiency.

### Isocitrate Dehydrogenase (IDH) Status

In some embodiments, the IDH mutation status of a cancer is used as a basis for selecting an individual for any treatment method disclosed herein. There exist a number of human IDH isozymes, which are involved in conversion of isocitrate, and are involved in, e.g., cellular metabolism, lipid synthesis, and oxidative respiration. For example, IDH isozymes include NADP⁺-dependent (e.g., IDH1 and IDH2) and NAD⁺-dependent (e.g., IDH3, which is composed of alpha, beta, and gamma subunits) IDH isozymes. IDH isozyme gene and protein sequences are known, e.g., IDH1 (UniProt O75874; GenBank Gene ID 3417), IDH2 (UniProt P48735; GenBank Gene ID 3418), and IDH3 (UniProt P51553, P50213, O43837; GenBank Gene ID 3419, 3420, 3421), which are hereby incorporated by reference in their entirety. In some embodiments, the presence of an IDH mutation, such as one or more mutations of one or more of IDH1, IDH2, and IDH3, or an indicator of the IDH mutation, is used as a basis for selecting an individual for any treatment disclosed herein.

In some embodiments, the IDH mutation status is based on an IDH mutation. In some embodiments, the IDH mutation status is based on two or more IDH mutations. In some embodiments, the IDH mutation status is based on two or more IDH mutations, wherein the two or more IDH mutations are any combination of mutations of the following: one or more IDH1 mutations, one or more IDH2 mutations, and one or more IDH3 mutations.

In some embodiments, the IDH mutation status is determined at any position of an IDH gene or an expression product thereof, such as RNA or protein. In some embodiments, the IDH mutation status is determined as compared to a nucleic acid encoding an IDH protein, such as a nucleic acid encoding IDH1, a nucleic acid encoding IDH2, or a nucleic acid encoding IDH3. In some embodiments, the IDH mutation status is determined as compared to an IDH protein, such as an IDH1 protein, an IDH2 protein, or an IDH3 protein. In some embodiments, the IDH mutation status is determined as compared to a known IDH nucleic acid or protein sequence, such as a wild type IDH1 sequence (e.g., as recorded in UniProt 075874), a wild type IDH2 sequence (e.g., as recorded in UniProt P48735), or a wild type IDH3 sequence (e.g., as recorded in UniProt P51553). In some embodiments, the IDH mutation status is determined as compared to a control, such as from an individual treated with a method disclosed herein or another individual. In some embodiments, the IDH mutation status is determined as compared to a control, such as non-cancerous tissue from an individual treated with a method disclosed herein or another individual. In some embodiments, the IDH mutation status is determined as compared to a single control. In some embodiments, the IDH mutation status is determined as compared to a plurality of controls, such as a population selected for a clinical trial.

In some embodiments, the IDH mutation is an IDH1 mutation. In some embodiments, the IDH1 mutation is an IDH1 mutation of the arginine 132 codon. In some embodiments, the IDH1 mutation of the arginine 132 codon is selected from the group consisting of: CGT>CAT, CGT>TGT, CGT>AGT, CGT>GGT, and CGT>CTT. In some embodiments, the IDH1 mutation is an IDH1 mutation of arginine 132. In some embodiments, the IDH1 mutation of arginine 132 is selected from the group consisting of: R132H, R132C, R132S, R132G, and R132L. In some embodiments, the IDH mutation is an IDH2 mutation. In some embodiments, the IDH2 mutation is an IDH2 mutation of the arginine 140 codon. In some embodiments, the IDH2 mutation of the arginine 140 codon is CGA>CAA. In some embodiments, the IDH2 mutation is an IDH2 mutation of the arginine 172 codon. In some embodiments, the IDH2 mutation of the arginine 172 codon is selected from the group consisting of: CGT>AAG, CGT>ATG, and CGT>TGG. In some embodiments, the IDH2 mutation is an IDH2 mutation of arginine 140. In some embodiments, the IDH2 mutation of arginine 140 is R140Q. In some embodiments, the IDH2 mutation is an IDH2 mutation of arginine 172. In some embodiments, the IDH2 mutation of arginine 172 is selected from the group consisting of: R172K, R172M, and R172W. In some embodiments, the IDH mutation is an IDH3 mutation. In some embodiments, the IDH mutation status is based on a mutation in D2HGDH and/or L2HGDH.

In some embodiments, the IDH mutation status of a cancer is based on an indicator of an IDH mutation, such as a level of an IDH substrate or metabolite. In some embodiments, the IDH mutation alters the normal activity range of an IDH isozyme, such as IDH1, IDH2, and IDH3. Generally, alteration of the normal activity range of an enzyme will impact the concentrations of substrates and metabolites of said enzyme. For example, a mutation of an enzyme may change the enzyme's affinity for a substrate and/or a metabolite, and thus alter the activity level of using or generating specific substrates or metabolites. Such activity levels may be determined based on levels of a substrate and/or a metabolite. In some embodiments, the IDH mutation reduces an activity level of an IDH isozyme (such as an activity level for using a specific substrate or producing a specific metabolite), as compared to a control (such as an activity level in a non-cancerous tissue of an individual or a known activity level of a non-mutated IDH isozyme). In some embodiments, the IDH mutation status of a cancer is based on a reduced activity level of an IDH isozyme, wherein the activity level of the IDH isozyme in the cancer is reduced by at least about 0.1-fold, such as at least about any of 0.25-fold, 0.5-fold, 0.75-fold, 1-fold, 2-fold, 5-fold, 10-fold, or 100-fold, as compared to a control. In some embodiments, the IDH mutation increases an activity level of an IDH isozyme (such as an activity level for using a specific substrate or producing a specific metabolite), as compared to a control (such as an activity level in a non-cancerous tissue of an individual or a known activity level of a non-mutated IDH isozyme). In some embodiments, the IDH mutation status of a cancer is based on an increased activity level of an IDH isozyme, wherein the activity level of the IDH isozyme in the cancer is increased by at least about 0.1-fold, such as at least about any of 0.25-fold, 0.5-fold, 0.75-fold, 1-fold, 2-fold, 5-fold, 10-fold, or 100-fold, as compared to a control. In some embodiments, the activity level of an IDH isozyme is based on one or more IDH isozyme substrates or metabolites, including isocitrate, NAD⁺, NADH, NADP⁺, NADPH, D-2-hydroxyglutarate, and 2-hydroxyglutarate.

In some embodiments, the methods disclosed herein further comprise assessing, such as determining, an IDH mutation status of a cancer of an individual. In some embodiments, assessing an IDH mutation status comprises determining a sequence of an IDH isozyme, such as IDH1, IDH2, and IDH3, or a portion thereof. In some embodiments, assessing an IDH mutation status comprises determining a DNA sequence of an IDH isozyme, or a portion thereof. In some embodiments, assessing an IDH mutation status comprises determining a RNA sequence of an IDH isozyme, or a portion thereof. In some embodiments, assessing an IDH mutation status comprises determining a protein sequence of an IDH isozyme, or a portion thereof. In some embodiments, assessing an IDH mutation status comprises detecting a gene or gene expression product of an IDH isozyme. In some embodiments, assessing an IDH mutation status comprises comparing a sequence of an IDH isozyme to a control (such as a sequence from an IDH isozyme from a non-cancerous tissue or a known sequence of an IDH isozyme). In some embodiments, assessing an IDH mutation status comprises determining the activity level of an IDH isozyme, such as IDH1, IDH2, and IDH3. In some embodiments, assessing an IDH mutation status comprises comparing an activity level of an IDH isozyme to a control (such as an activity level of an IDH isozyme from a non-cancerous tissue or a known activity level of an IDH isozyme). In some embodiments, assessing an IDH mutation status comprises comparing an expression level of an IDH isozyme to a control (such as an expression level of an IDH isozyme from a non-cancerous tissue or a known expression level of an IDH isozyme).

In some embodiments, the IDH mutation status of a cancer is determined prior to administration of an agent disclosed in the methods described herein. For example, in some embodiments, the IDH mutation status of a cancer is determined prior to administration of an effective amount of a hypoxia targeting composition (such as a hypoxia-activated drug or a prodrug thereof). In some embodiments, the IDH mutation status of a cancer is determined prior to administration of (i) an effective amount of a hypoxia targeting composition (such as a hypoxia-activated drug or a prodrug thereof), and (ii) an effective amount of a PARP inhibitor. In some embodiments, the IDH mutation status of a cancer is further evaluated during or after administration of an agent disclosed in the methods described herein.

The IDH mutation status may be assessed, such as determined, by methods known in the art. See, e.g., Franca et al., Q Rev Biophy, 2002, 35, 169-200; and Steen et al., Nat Rev Mol Cell Biol, 2004, 5, 699-711, which are hereby incorporated by reference in their entirety. In some embodiments, the IDH mutation status is determined by sequence analysis, for example by sequencing analysis and/or detection of genomic DNA, or expression products therefrom (such as RNA or protein), of a sample obtained from an individual. In some embodiments, the IDH mutation status of the cancer is based on one or more of: DNA sequencing, DNA sequence detection, RNA sequencing, RNA transcript detection, protein sequencing, and protein detection. Methods for sequencing and/or detecting a gene and a gene expression product are well known in the art and include, but are not limited to, a high-throughput DNA sequencing method, Massively Parallel Signature Sequencing (MPSS), polony sequencing, pyrosequencing, SOLid sequencing, nanopore sequencing, immunological assays, nuclease protection assays, northern blots, in situ hybridization, ELISA, reverse transcriptase Polymerase Chain Reaction (RT-PCR), Real-Time Polymerase Chain Reaction, expressed sequence tag (EST) sequencing, cDNA microarray hybridization or gene chip analysis, subtractive cloning, Serial Analysis of Gene Expression (SAGE), Sequencing-By-Synthesis (SBS), aptamer-based assays, western blot, enzyme immunoassays, Luminex Platform utilizing color, and mass spectrometry. In some embodiments, the IDH mutation status is based on determining an IDH gene sequence, such as a gene sequence of any one of IDH1, IDH2, and IDH3, or a portion thereof. In some embodiments, the IDH mutation status is based on determining an IDH RNA sequence (e.g., mRNA), such as a RNA sequence of any one of IDH1, IDH2, and IDH3, or a portion thereof. In some embodiments, the IDH mutation status is based on determining an IDH protein sequence, such as a protein sequence of any one of IDH1, IDH2, and IDH3, or a portion thereof. In some embodiments, the IDH mutation status is determined by metabolite profiling, for example by measuring an abundance of a substrate and/or a metabolite in a sample obtained from an individual. Methods for measuring an abundance of a substrate and/or a metabolite are well known in the art and include, but are not limited to, flux measurements and mass spectrometry.

In some embodiments, any of the methods disclosed herein further comprise selecting an individual for treatment based on an IDH mutation status of the cancer. In some embodiments, any of the methods disclosed herein further comprise selecting an individual for treatment based on an IDH mutation status of a cancer, wherein the IDH mutation status of the cancer is based on the presence of one or more mutations of one or more of IDH1, IDH2, and IDH3, or an indicator of the IDH mutation (such as an enzyme activity level).

### Hypoxia Status

In some embodiments, the hypoxia status of a cancer is used as a basis for selecting an individual for treatment with any one of the methods disclosed herein. Hypoxia is characterized by a reduced oxygenation level in a tissue (such as a cell), as compared to normoxia (oxygenation level of about 20% to about 21% oxygen) and/or physoxia, the oxygenation level in normal, healthy tissue. S R McKeown, Br J Radiol, 87, 2014. Physoxic oxygenation concentrations vary in different tissue types, locations within the same tissue, and temporally. There are numerous approaches for assessing (such as identifying) a hypoxia status, such as a hypoxic conditions in a tissue, e.g., as determined based on measuring oxygen concentrations and/or hypoxia-related gene expression.

In some embodiments, the hypoxia status indicates presence of hypoxia in at least a portion (e.g., at least one cell) of a cancer. In some embodiments, presence of hypoxia in at least a portion of a cancer is based on the portion of the cancer having a tissue oxygenation level of about 5% to about 0.01% oxygen, such as any of about 4.2% to about 0.2% oxygen, about 3% to about 0.2% oxygen, about 2.5% to about 0.2% oxygen, or about 2% to about 0.3% oxygen. In some embodiments, presence of hypoxia in at least a portion of a cancer is based on the portion of the cancer having a tissue oxygenation level of about 5% oxygen or less, such as about any of 4.75% oxygen or less, 4.5% oxygen or less, 4.25% oxygen or less, 4% oxygen or less, 3.75% oxygen or less, 3.5% oxygen or less, 3.25% oxygen or less, 3% oxygen or less, 2.75% oxygen or less, 2.5% oxygen or less, 2.25% oxygen or less, 2% oxygen or less, 1.75% oxygen or less, 1.5% oxygen or less, 1.25% oxygen or less, 1% oxygen or less, 0.75% oxygen or less, 0.5% oxygen or less, or 0.25% oxygen or less.

In some embodiments, the hypoxia status indicates the presence of hypoxia in at least a portion of a cancer, wherein the hypoxia status is based on a low tissue oxygenation level in at least the portion of the cancer. In some embodiments, the low tissue oxygenation level is at least a portion of a cancer having a tissue oxygenation level of about 5% oxygen or less, such as about any of 4.75% oxygen or less, 4.5% oxygen or less, 4.25% oxygen or less, 4% oxygen or less, 3.75% oxygen or less, 3.5% oxygen or less, 3.25% oxygen or less, 3% oxygen or less, 2.75% oxygen or less, 2.5% oxygen or less, 2.25% oxygen or less, 2% oxygen or less, 1.75% oxygen or less, 1.5% oxygen or less, 1.25% oxygen or less, 1% oxygen or less, 0.75% oxygen or less, 0.5% oxygen or less, or 0.25% oxygen or less.

Methods for assessing (such as determining or measuring) a level of tissue oxygenation in a cancer are known in the field. For example, the level of tissue oxygenation can be determined using an oxymetric technique, such as a needle electrode technique, such as an Eppendorf electrode technique, a positron-emission tomography (PET) technique, such as a ¹⁸F-Fluoromisonidazole PET technique, a magnetic resonance imaging (MM) technique, such as a dynamic contrast-enhanced MRI technique, a ¹H relaxation imaging technique, an electron paramagnetic resonance technique, a single-photon emission computed tomography technique, or any combination thereof. See Colliez F. et al., Front Oncol, 7, 2017; and Walsh et al., Antioxid Redox Signal, 21, 2014. In some embodiments, the hypoxia status of a cancer is determined based on assessing oxygenation level of at least a portion of the cancer using an oxymetric technique, wherein the oxymetric technique comprises one or more of an Eppendorf electrode technique, a PET technique, a MM technique, a ¹H relaxation imaging technique, and an electron paramagnetic resonance technique.

In some embodiments, the hypoxia status, such as presence of hypoxia, in at least a portion of a cancer is based on the portion of the cancer having a hypoxia biomarker profile. In some embodiments, the hypoxia biomarker profile comprises one or more hypoxia biomarker(s), such as a gene product, such as RNA or protein, that is expressed due to hypoxic conditions. In some embodiments, the hypoxia biomarker profile comprises an endogenous biomarker. In some embodiments, the hypoxia biomarker is an endogenous biomarker. In some embodiments, the hypoxia biomarker is a secreted hypoxia biomarker. Hypoxia biomarkers and hypoxia biomarker profiles are known in the field. See Le, Q.-T. et al., Cancer Metastasis Rev, 27, 2008; Walsh et al., Antioxid Redox Signal, 21, 2014; Bruna, A. et al., Cell, 167, 2016; Buffa, F M et al., Br J Cancer, 102, 2010; and El Guerrab, A. et al., PLOS One, 12, 2017. In some embodiments, the hypoxia biomarker profile comprises one or more of hypoxia inducible factor-1 (HIF-1), hypoxia inducible factor-2 (HIF-2), glucose transporter-1 (Glut-1), CA IX, vascular endothelial growth factor (VEGF), Bcl-2/adenovirus E1B 19 kD-interacting enzyme (BNIP3), lysyl oxidase (LOX), lactate dehydrogenase isoenzyme-5 (LDH-5), plasminogen activator inhibitor-1 (PAM), galectin-1, and osteopontin (OPN). Methods for determining hypoxia biomarker profiles are known in the art, such as those disclosed herein and in the references cited above.

In some embodiments, the hypoxia status, such as presence of hypoxia, in at least a portion of a cancer is determined via a hypoxia stain, such as a chemical stain or an immuno-stain. In some embodiments, the hypoxia status, such as presence of hypoxia, in at least a portion of a cancer is determined via a tissue staining technique. In some embodiments, the reagent used in a hypoxia staining technique is pimonidazole.

In some embodiments, the hypoxia status, such as presence of hypoxia, in at least a portion of a cancer is determined via a hypoxia score. For example, hypoxia scores known in the art may be used with the methods described herein, e.g., those disclosed in Buffa, F M, Br J Cancer, 19, 2010, 428-35.

In some embodiments, the hypoxia status of a cancer (or a portion thereof) is determined prior to administration of an effective amount of a hypoxia targeting composition (such as a hypoxia-activated drug or a prodrug thereof). In some embodiments, the hypoxia status of a cancer (or a portion thereof) is determined prior to administration of an effective amount of a PARP inhibitor. In some embodiments, the hypoxia status of a cancer (or a portion thereof) is determined prior to administration of: (i) an effective amount of a hypoxia targeting composition (such as a hypoxia-activated drug or a prodrug thereof), and (ii) an effective amount of a PARP inhibitor.

In some embodiments, any of the methods disclosed herein further comprise selecting an individual for treatment based on a hypoxia status of a cancer. In some embodiments, any of the methods disclosed herein further comprise selecting an individual for treatment based on a hypoxia status, wherein the hypoxia status of a cancer is based on a low tissue oxygenation level in at least the portion of the cancer.

Hypoxia may be highly heterogeneous, both spatially and temporally, within and between tumors. Thus, in some embodiments, the hypoxia status is based on more than one assessment of a cancer performed at one or more times, such as one or more times prior to administration of (i) an effective amount of a hypoxia targeting composition (such as a hypoxia-activated drug or a prodrug thereof), and/or (ii) an effective amount of a PARP inhibitor. In some embodiments, the hypoxia status is determined based on any one of the assessments of a cancer (e.g., hypoxia status is based on a single measurement indicating the presence of hypoxia in the cancer or a portion thereof).

The bases for selecting an individual for a treatment method disclosed herein, such as an HR deficiency status, an IDH mutation status, and a hypoxia status, are determined via a sample (e.g., a sample from an individual or a reference sample from the individual or another) obtained from one or more individuals. One of ordinary skill in the art will understand the sample(s) needed to assess the bases for selecting an individual for a treatment method disclosed herein. In some embodiments, the sample comprises a tumor tissue, a primary tumor tissue, a metastatic tumor tissue, a normal tissue, such as a normal tissue adjacent to a tumor tissue or a normal tissue distal to a tumor tissue, a blood sample, or other biological sample. In some embodiments, the sample is a biopsy containing cancer cells or components thereof, such as excretions. In some embodiments, the biopsy is a fine needle aspiration of a tumor tissue. In some embodiments, the biopsy is a laparoscopically obtained sample. In some embodiments, a biopsy is taken to determine whether an individual has a cancer and is subsequently used as a sample for the methods disclosed herein. In some embodiments, the sample is a surgically obtained sample. In some embodiments, the sample comprises a circulating cancer cell, such as a cancerous blood cell or a metastatic cell. In some embodiments, the circulating cancer cell is a cell that has detached from a tumor. In some embodiments, a sample may be obtained at a different time than when the sample is analyzed for the methods disclosed herein, such as using a frozen tissue sample from an individual.

The bases disclosed herein for use with the methods of the present application, such as HR deficiency status, IDH mutation status, and hypoxia status, may, in some aspects, may be based on a comparison to a control. In some embodiments, control is a known standard obtained from the literature (e.g., a known gene sequence, RNA sequence, protein sequence, gene expression level, enzyme activity level, tissue oxygenation level, or substrate and/or metabolite levels). In some embodiments, the control is a control sample obtained from the individual to be, or being, treated using the methods disclosed herein (e.g., a control sample from a non-cancerous tissue). In some embodiments, the control is a control sample obtained from an individual other than the individual to be, or being, treated using the methods disclosed herein (e.g., a control sample from a healthy volunteer or a volunteer not having cancer). In some embodiments, the control is obtained from a given patient population. For example, regarding a level of gene expression or enzyme activity level, a control level may be the median expression level of that gene or the median enzyme activity level of that enzyme for the patient population. And, for example, if the expression level of a gene of interest for the single patient is determined to be above the median expression level of the patient population, that patient is determined to have high expression of the gene of interest. Alternatively, if the expression level of a gene of interest for the single patient is determined to be below the median expression level of the patient population, that patient is determined to have low expression of the gene of interest. In some embodiments, the single patient has a disease (such as cancer) and the patient population does not have the disease. In some embodiments, the single patient and the patient population have the same histological type of a disease. A population may be about, or alternatively at least about any of the following, in terms of number of individuals measured: 2, 5, 10, 15, 20, 25, 30, 50, 60, 75, 100, 125, 150, 175, 200, 225, 250, 300, 400, 500. Preferably, a sufficient number of individuals are measured to provide a statistically significant population, which can be determined by methods known in the art. In some embodiments, the population is a group participating in a clinical trial.

### Hypoxia Targeting Compositions

In some embodiments, methods disclosed herein comprise administering an effective amount of a hypoxia targeting composition (such as a hypoxia-activated drug or a prodrug thereof). In some embodiments, the method comprises administering an effective amount of a hypoxia-activated drug. In some embodiments, the method comprises administering an effective amount of a hypoxia-activated drug prodrug.

In some embodiments, the hypoxia targeting composition is a hypoxia-activated drug or a prodrug thereof.

Hypoxia-activated drugs or prodrugs thereof, including compounds known as bioreductive drugs, are compounds that are chemically converted, such as via chemical reduction, at low oxygen levels (e.g., hypoxic condition such as an oxygen (O₂) concentration of 2% or less) to the desired active compound. E.g., Mistry, I. N. et al., Int J Radiat Oncol Biol Phys, 98, 2017. In some embodiments, the hypoxia-activated drug or a prodrug thereof is selected from one of the following chemical classes of hypoxia-activated drugs or prodrugs thereof: nitro compounds, quinones, aromatic N-oxides, aliphatic N-oxides, and transition metal complexes.

In some embodiments, the hypoxia-activated drug or a prodrug thereof is selected from the group consisting of: apaziquone (E09), AQ4N, etanidazole, evofosfamide (TH-302), mitomycin C, nimorazole, pimonidazole, porfiromycin, PR-104, SN30000, tarloxotinib, and tirapazamine. In some embodiments, the hypoxia-activated drug or the prodrug thereof is apaziquone (E09), AQ4N, etanidazole, evofosfamide (TH-302), mitomycin C, nimorazole, pimonidazole, porfiromycin, PR-104, SN30000, tarloxotinib, or tirapazamine, or an analog or derivative of the hypoxia-activated drug or the prodrug thereof. Analogs and derivatives of a hypoxia-activated drug or a prodrug thereof include, but are not limited to, compounds that are structurally similar to the hypoxia-derivatives or the prodrug thereof, and/or are in the same general chemical class as the hypoxia-derivatives or the prodrug thereof. In some embodiments, the analog or derivative of a hypoxia-activated drug or a prodrug thereof retains one or more similar biological, pharmacological, chemical, and/or physical property (including, for example, functionality).

In some embodiments, the hypoxia-activated drug or a prodrug thereof is tirapazamine. Tirapazamine is also referred to as triazone, SR-4233, WIN59075, SR259075, 3-amino-1,2,4-benzotriazine-1,4-dioxide. In some embodiments, the hypoxia-activated drug or a prodrug thereof is an analog or derivative of tirapazamine. In some embodiments, the analog or derivative of tirapazamine is SN30000. In some embodiments, the analog or derivative of tirapazamine is SN29751. In some embodiments, the hypoxia-activated drug or a prodrug thereof is SN30000 or an analog or derivative thereof. In some embodiments, the hypoxia-activated drug or a prodrug thereof is SN29751 or an analog or derivative thereof.

In some embodiments, the hypoxia-activated drug or a prodrug thereof, upon activation, leads to generation of a radical. In some embodiments, the hypoxia-activated drug or prodrug thereof is not a molecular targeting compound (e.g., a compound that inhibits a specific enzyme). In some embodiments, the hypoxia-activated drug or prodrug thereof is a molecular targeting compound (e.g., a compound that inhibits a specific enzyme).

In some embodiments, the hypoxia targeting composition selectively targets (such as inhibits) a hypoxic cell via, e.g., a biomarker of hypoxia, such as a protein that is overexpressed in the hypoxic cell. In some embodiments, the hypoxia targeting composition selectively targets ataxia-telangiectasia mutated protein kinase (ATM), ataxia telangiectasia and Rad3-related protein (ATR), Bcl-2/adenovirus E1B 19 kD-interacting enzyme (BNIP3), CA IX, DNA-dependent protein kinase (DNA-PK), galectin-1, glucose transporter-1 (Glut-1), hypoxia inducible factor-1 (HIF-1), hypoxia inducible factor-2 (HIF-2), lactate dehydrogenase isoenzyme-5 (LDH-5), lysyl oxidase (LOX), the MRN complex or a component thereof (such as Mre11, Rad50, and Nbs1), osteopontin (OPN), plasminogen activator inhibitor-1 (PAI-I), or vascular endothelial growth factor (VEGF). In some embodiments, the hypoxia targeting composition is an inhibitor of a biomarker of hypoxia, such as a protein that is overexpressed in the hypoxic cell. In some embodiments, the hypoxia targeting composition is an ATM inhibitor, ATR inhibitor, BNIP3 inhibitor, CA IX inhibitor, DNA-PK inhibitor, galectin-1 inhibitor, Glut-1 inhibitor, HIF-1 inhibitor, HIF-2 inhibitor, LDH-5 inhibitor, LOX inhibitor, MRN inhibitor or an inhibitor of a component thereof, OPN inhibitor, PAI-I inhibitor, or VEGF inhibitor. In some embodiments, the hypoxia targeting composition is an ATM inhibitor, ATR inhibitor, BNIP3 inhibitor, CA IX inhibitor, DNA-PK inhibitor, galectin-1 inhibitor, Glut-1 inhibitor, HIF-1 inhibitor, HIF-2 inhibitor, LDH-5 inhibitor, LOX inhibitor, MRN inhibitor or an inhibitor of a component thereof, OPN inhibitor, PAI-I inhibitor, or VEGF inhibitor, wherein the hypoxia targeting composition is a hypoxia-activated drug or a prodrug thereof.

The hypoxia targeting composition (such as the hypoxia-activated drug or the prodrug thereof) may be formulated for a desired administration route to an individual. In some embodiments, the hypoxia targeting composition (such as the hypoxia-activated drug or the prodrug thereof) is suitable for administration to an individual (such as human) via various routes, including, for example, parenteral, intravenous, intraventricular, intra-arterial, intraperitoneal, intrapulmonary, oral, inhalation, intravesicular, intramuscular, intra-tracheal, subcutaneous, intraocular, intrathecal, transmucosal, and transdermal administration. Accordingly, in some embodiments, the hypoxia targeting composition (such as the hypoxia-activated drug or the prodrug thereof) is a pharmaceutically acceptable salt of the hypoxia targeting composition. In some embodiments, the hypoxia targeting composition (such as the hypoxia-activated drug or the prodrug thereof) is formulated with one or more pharmaceutically acceptable carriers and/or excipients. In some embodiments, the hypoxia targeting composition (such as the hypoxia-activated drug or the prodrug thereof) is suitable for (e.g., formulated for) oral administration. In some embodiments, the hypoxia-activated drug or the prodrug thereof is suitable for oral administration. In some embodiments, tirapazamine is suitable for oral administration.

In some embodiments, the effective amount of a hypoxia targeting composition (such as a hypoxia-activated drug or a prodrug thereof) is about 0.1 mg to 1000 mg, such as about 1 mg to about 500 mg, about 20 mg to about 400 mg, or about 100 mg to about 400 mg. In some embodiments, the effective amount of a hypoxia targeting composition (such as a hypoxia-activated drug or a prodrug thereof) is at least about 0.1 mg, such as at least about any of 1 mg, 5 mg, 10 mg, 25 mg, 50 mg, 75 mg, 100 mg, 150 mg, 200 mg, 250 mg, 300 mg, 350 mg, 400 mg, 450 mg, 500 mg, 550 mg, 575 mg, 600 mg, 650 mg, 700 mg, 750 mg, 800 mg, 850 mg, 900 mg, 950 mg, or 1000 mg. In some embodiments, the effective amount of a hypoxia targeting composition (such as a hypoxia-activated drug or a prodrug thereof) is no greater than about 1000 mg, such as no greater than about any of 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, 100 mg, 75 mg, 50 mg, 25 mg, 10 mg, 5 mg, or 1 mg.

### PARP Inhibitors

In some embodiments, methods disclosed herein comprise administering an effective amount of a PARP inhibitor. PARP inhibitors encompassed by the present disclosure include pharmaceutically acceptable compositions that inhibit the activity of the enzyme, poly(ADP-ribose) polymerase (PARP).

In some embodiments, the PARP inhibitor is selected from the group consisting of: 3-aminobenzamine, BGD-290, CEP 9722, E7016, iniparib, niraparib, olaparib, rucaparib, talazoparib, and veliparib. In some embodiments, the PARP inhibitor is 3-aminobenzamine, BGD-290, CEP 9722, E7016, iniparib, niraparib, olaparib, rucaparib, talazoparib, Fluzoparib, or veliparib, or an analog or derivative thereof. In some embodiments, the PARP inhibitor is olaparib. In some embodiments, the PARP inhibitor is talazoparib. In some embodiments, the PARP inhibitor is rucaparib. In some embodiments, the PARP inhibitor is niraparib.

In some embodiments, the effective amount of the PARP inhibitor is about 20 mg to about 2000 mg, such as about 100 mg to about 1000 mg, about 300 mg to about 600 mg, or about 300 mg to about 1500 mg. In some embodiments, the effective amount of a PARP inhibitor is at least about 20 mg, such as at least about any of 25 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the effective amount of a PARP inhibitor is no greater than about 2000 mg, such as no greater than about any of 1950 mg, 1900 mg, 1850 mg, 1800 mg, 1750 mg, 1700 mg, 1650 mg, 1600 mg, 1550 mg, 1500 mg, 1450 mg, 1400 mg, 1350 mg, 1300 mg, 1250 mg, 1200 mg, 1150 mg, 1100 mg, 1050 mg, 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, 100 mg, 75 mg, 50 mg, or 25 mg.

### Modes of Administration

In some embodiments, the methods described herein comprise administration of: (i) an effective amount of a hypoxia targeting composition (such as a hypoxia-activated drug or a prodrug thereof), and (ii) an effective amount of a PARP inhibitor.

In some embodiments, (i) the effective amount of the hypoxia targeting composition (such as the hypoxia-activated drug or the prodrug thereof), and (ii) the effective amount of the PARP inhibitor are administered simultaneously. When a hypoxia targeting composition (such as a hypoxia-activated drug or a prodrug thereof) and a PARP inhibitor are administered simultaneously, the hypoxia targeting composition (such as the hypoxia-activated drug or the prodrug thereof) and the PARP inhibitor may be contained in the same composition or in separate composition.

In some embodiments, (i) the effective amount of the hypoxia targeting composition (such as the hypoxia-activated drug or the prodrug thereof), and (ii) the effective amount of the PARP inhibitor are administered sequentially. When a hypoxia targeting composition (such as a hypoxia-activated drug or a prodrug thereof) and a PARP inhibitor are administered sequentially, either drug may be administered first. For example, in some embodiments, the effective amount of the hypoxia targeting composition (such as the hypoxia-activated drug or the prodrug thereof) is administered before an effective amount of a PARP inhibitor. For example, in some embodiments, the effective amount of the PARP inhibitor is administered before an effective amount of a hypoxia targeting composition (such as a hypoxia-activated drug or a prodrug thereof).

In some embodiments, (i) the effective amount of the hypoxia targeting composition (such as the hypoxia-activated drug or the prodrug thereof), and (ii) the effective amount of the PARP inhibitor are administered concurrently. In some embodiments, when a hypoxia targeting composition (such as a hypoxia-activated drug or a prodrug thereof) and a PARP inhibitor are administered concurrently, the administration period of a hypoxia targeting composition (such as a hypoxia-activated drug or a prodrug thereof) overlaps with the administration of a PARP inhibitor.

In some embodiments, the dosing frequency and or dosage amount of a hypoxia targeting composition (such as a hypoxia-activated drug or a prodrug thereof) and/or a PARP inhibitor are adjusted over the course of the treatment, based on the judgment of the administering physician. When administered separately, a hypoxia targeting composition (such as a hypoxia-activated drug or a prodrug thereof) may be administered at different dosing frequency or intervals than for a PARP inhibitor. In some embodiments, for combination treatments disclosed herein the effective amount of the hypoxia targeting composition (such as the hypoxia-activated drug or the prodrug thereof), and/or (ii) the effective amount of the PARP inhibitor is/are a lower dose than for a treatment method wherein only one of the effective amount of the hypoxia targeting composition (such as the hypoxia-activated drug or the prodrug thereof) or (ii) the effective amount of the PARP inhibitor is administered to an individual.

In some embodiments, the hypoxia targeting composition (such as the hypoxia-activated drug or the prodrug thereof) and a PARP inhibitor are administered using the same route of administration. In some embodiments, the hypoxia targeting composition (such as the hypoxia-activated drug or the prodrug thereof) and a PARP inhibitor are administered using a different same route of administration. For example, the agents described herein can be administered to an individual (such as human) via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. In some embodiments, the hypoxia targeting composition (such as the hypoxia-activated drug or the prodrug thereof) is administered via oral administration.

The methods disclosed herein may be performed for any number of treatment cycles. In some embodiments, the individual is treated for at least about any of one, two, three, four, five, six, seven, eight, nine, or ten treatment cycles.

### Exemplary Cancers

The methods disclosed herein are useful for treating a proliferative disease, such as a cancer, in an individual.

In some embodiments, the cancer is a solid tumor. In some embodiments, the cancer is a hematopoietic malignancy.

In some embodiments, the cancer is a breast cancer (such as triple negative breast cancer), ovarian cancer, pancreatic cancer, fibrosarcoma, head and neck cancer, prostate cancer, glioma, glioblastoma, astrocytoma, oligodendroglioma, leukemia (such as B-acute lymphoid leukemia), colorectal cancer, oligoastrocytoma, neuroectodermal tumor, myeloid cancer (such as acute myeloid leukemia (AML)), or lung cancer (such as non-small cell lung cancer).

In some embodiments, the cancer is a HR deficient cancer. In some embodiments, the cancer is a HR deficient cancer, wherein the cancer comprises a mutation in BRCA1. In some embodiments, the cancer is a HR deficient cancer, wherein the cancer comprises a mutation in BRCA2. In some embodiments, the cancer is a HR deficient cancer, wherein the cancer comprises a mutation in RAD51. In some embodiments, the cancer is a HR deficient cancer, wherein the cancer comprises a mutation in XRCC3. In some embodiments, the cancer is a BRCA1 mutant cancer. In some embodiments, the cancer is a BRCA2 mutant cancer. In some embodiments, the cancer is a RAD51 mutant cancer. In some embodiments, the cancer is a XRCC3 mutant cancer.

In some embodiments, the cancer is an IDH mutant cancer. In some embodiments, the cancer is an IDH mutant cancer, wherein the cancer comprises a mutation in IDH1. In some embodiments, the cancer is an IDH mutant cancer, wherein the cancer comprises a mutation in IDH2. In some embodiments, the cancer is an IDH mutant cancer, wherein the cancer comprises a mutation in IDH3. In some embodiments, the cancer is an IDH1 mutant cancer. In some embodiments, the cancer is an IDH2 mutant cancer. In some embodiments, the cancer is an IDH3 mutant cancer.

In some embodiments, the cancer is a hypoxic cancer. In some embodiments, the cancer is a hypoxic cancer comprising a 1-electron and/or 2-electron reductase.

In some embodiments, the cancer is an early stage cancer, a non-metastatic cancer, a primary cancer, an advanced cancer, a locally advanced cancer, a metastatic cancer, a cancer in remission, a recurrent cancer, a resistant cancer, or a refractory cancer. In some embodiments, the cancer is a localized respectable cancer (e.g., a tumor that is confined to a portion of an organ that allows for complete surgical removal), a localized unresectable cancer (e.g., a localized tumor that is unresectable because crucial blood vessel structures), or an unresectable cancer. In some embodiments, the cancer is, according to TNM classifications, a stage I tumor, a stage II tumor, a stage III tumor, a stage IV tumor, a N1 tumor, or a M1 tumor.

### Exemplary Embodiments of the Methods Disclosed Herein

A method of treating a cancer in an individual, according to the disclosure provided herein, may be any combination of aspects of the present application. For example, in some embodiments, there is provided a method for treating a breast cancer in an individual in need thereof, the method comprising administering to the individual an effective amount of tirapazamine, wherein a HR deficiency status of the breast cancer is used as a basis for selecting the individual for treatment. In some embodiments, the method for treating a breast cancer in an individual comprises selecting the individual for treatment based on a positive status indicative of HR deficiency in the breast cancer or a portion thereof. In some embodiments, the present application provides methods of selecting (including identifying) an individual having a breast cancer suitable for treatment with the methods disclosed herein, wherein the method comprises determining a HR deficiency status of the breast cancer in the individual. In some embodiments, the present application provides methods of selecting (including identifying) an individual having a breast cancer suitable for treatment with the methods disclosed herein, wherein the method comprises determining a HR deficiency status of the breast cancer in the individual, and wherein the individual is selected if the individual has a positive status indicative of HR deficiency in the breast cancer or a portion thereof. In some embodiments, the HR deficiency status of the breast cancer is based on a HR deficiency signature. In some embodiments, the HR deficiency status of a breast cancer is based on one or more of the following: (i) a sequence of a gene or a product thereof; (ii) telomeric allelic imbalance (TAI); (iii) large-scale state transitions (LST); (iv) loss of heterozygosity (LOH); and (v) promoter methylation (or lack thereof). In some embodiments, the HR deficiency status of a breast cancer is determined based on DNA sequencing of one or more genes, or a portion thereof. In some embodiments, the HR deficiency status of a breast cancer is determined based on RNA sequencing of one or more genes transcripts, e.g., mRNA, or a portion thereof. In some embodiments, the HR deficiency status of a breast cancer is determined based on protein sequencing of one or more gene products, or a portion thereof. In some embodiments, the HR deficiency status of a breast cancer is determined based on one or more of the following: (i) assessing a gene sequence or a product thereof; (ii) assessing loss of heterozygosity (LOH); (iii) assessing telomeric allelic imbalance (TAI); (iv) assessing large-scale state transitions (LST); and (v) assessing promoter methylation (or lack thereof). In some embodiments, the HR deficiency status of the breast cancer is determined prior to administration of an effective amount of tirapazamine. In some embodiments, the methods disclosed herein further comprise determining a HR deficiency status of a breast cancer prior to administration of an effective amount of tirapazamine. In some embodiments, the methods disclosed herein further comprise selecting an individual for treatment based on a HR deficiency status of a breast cancer. In some embodiments, the IDH mutation status of a breast cancer is further used as a basis for selecting the individual for treatment. In some embodiments, the hypoxia status of a breast cancer is further used as a basis for selecting the individual for treatment.

In some embodiments, there is provided a method for treating a breast cancer in an individual in need thereof, the method comprising administering to the individual an effective amount of tirapazamine, wherein a hypoxia status of the breast cancer is used as a basis for selecting the individual for treatment. In some embodiments, the present application provides methods for treating a breast cancer in an individual having hypoxia in the breast cancer or a portion thereof. In some embodiments, the method for treating a breast cancer in an individual comprises selecting the individual for treatment based on a hypoxia status indicative of the breast cancer or a portion thereof being hypoxic. In some embodiments, the present application provides methods of selecting (including identifying) an individual having a breast cancer suitable for treatment with the methods disclosed herein, wherein the method comprises determining a hypoxia status of the breast cancer in the individual. In some embodiments, the present application provides methods of selecting (including identifying) an individual having a breast cancer suitable for treatment with the methods disclosed herein, wherein the method comprises determining a hypoxia status of the breast cancer in the individual, and wherein the individual is selected if the individual has a hypoxia status indicative of the breast cancer or a portion thereof being hypoxic. In some embodiments, the hypoxia status of a breast cancer is based on one or more of the following: (i) a tissue oxygenation level; and (ii) a hypoxia biomarker. In some embodiments, the hypoxia status of a breast cancer is based on a low tissue oxygenation level. In some embodiments, the low tissue oxygenation level is a tissue oxygenation level of about 4% or less of oxygen, such as about 3% or less of oxygen, about 2% or less of oxygen, or about 1% or less of oxygen. In some embodiments, the tissue oxygenation level is based on an oxygenation level obtained via an oxymetric technique. In some embodiments, the hypoxia status of the breast cancer is determined based on one or more of the following: (i) assessing a tissue oxygenation level, such as via an oxymetric technique; and (ii) assessing a hypoxia biomarker. In some embodiments, the hypoxia status of a breast cancer is determined prior to administration of an effective amount of tirapazamine. In some embodiments, the methods disclosed herein further comprise determining a hypoxia status of a breast cancer prior to administration of an effective amount of tirapazamine. In some embodiments, the method further comprises selecting the individual for treatment based on the hypoxia status of the breast cancer. In some embodiments, the HR deficiency status of a breast cancer is further used as a basis for selecting the individual for treatment. In some embodiments, the IDH mutation status of a breast cancer is further used as a basis for selecting the individual for treatment.

In some embodiments, there is provided a method for treating an ovarian cancer in an individual in need thereof, the method comprising administering to the individual an effective amount of tirapazamine, wherein a HR deficiency status of the ovarian cancer is used as a basis for selecting the individual for treatment. In some embodiments, the method for treating an ovarian cancer in an individual comprises selecting the individual for treatment based on a positive status indicative of HR deficiency in the ovarian cancer or a portion thereof. In some embodiments, the present application provides methods of selecting (including identifying) an individual having an ovarian cancer suitable for treatment with the methods disclosed herein, wherein the method comprises determining a HR deficiency status of the ovarian cancer in the individual. In some embodiments, the present application provides methods of selecting (including identifying) an individual having an ovarian cancer suitable for treatment with the methods disclosed herein, wherein the method comprises determining a HR deficiency status of the ovarian cancer in the individual, and wherein the individual is selected if the individual has a positive status indicative of HR deficiency in the ovarian cancer or a portion thereof. In some embodiments, the HR deficiency status of the ovarian cancer is based on a HR deficiency signature. In some embodiments, the HR deficiency status of an ovarian cancer is based on one or more of the following: (i) a sequence of a gene or a product thereof (ii) telomeric allelic imbalance (TAI); (iii) large-scale state transitions (LST); (iv) loss of heterozygosity (LOH); and (v) promoter methylation (or lack thereof). In some embodiments, the HR deficiency status of an ovarian cancer is determined based on DNA sequencing of one or more genes, or a portion thereof. In some embodiments, the HR deficiency status of an ovarian cancer is determined based on RNA sequencing of one or more genes transcripts, e.g., mRNA, or a portion thereof. In some embodiments, the HR deficiency status of an ovarian cancer is determined based on protein sequencing of one or more gene products, or a portion thereof. In some embodiments, the HR deficiency status of an ovarian cancer is determined based on one or more of the following: (i) assessing a gene sequence or a product thereof; (ii) assessing loss of heterozygosity (LOH); (iii) assessing telomeric allelic imbalance (TAI); (iv) assessing large-scale state transitions (LST); and (v) assessing promoter methylation (or lack thereof). In some embodiments, the HR deficiency status of the ovarian cancer is determined prior to administration of an effective amount of tirapazamine. In some embodiments, the methods disclosed herein further comprise determining a HR deficiency status of an ovarian cancer prior to administration of an effective amount of tirapazamine. In some embodiments, the methods disclosed herein further comprise selecting an individual for treatment based on a HR deficiency status of an ovarian cancer. In some embodiments, the IDH mutation status of an ovarian cancer is further used as a basis for selecting the individual for treatment. In some embodiments, the hypoxia status of an ovarian cancer is further used as a basis for selecting the individual for treatment.

In some embodiments, there is provided a method for treating an ovarian cancer in an individual in need thereof, the method comprising administering to the individual an effective amount of tirapazamine, wherein a hypoxia status of the ovarian cancer is used as a basis for selecting the individual for treatment. In some embodiments, the present application provides methods for treating an ovarian cancer in an individual having hypoxia in the ovarian cancer or a portion thereof. In some embodiments, the method for treating an ovarian cancer in an individual comprises selecting the individual for treatment based on a hypoxia status indicative of the ovarian cancer or a portion thereof being hypoxic. In some embodiments, the present application provides methods of selecting (including identifying) an individual having an ovarian cancer suitable for treatment with the methods disclosed herein, wherein the method comprises determining a hypoxia status of the ovarian cancer in the individual. In some embodiments, the present application provides methods of selecting (including identifying) an individual having an ovarian cancer suitable for treatment with the methods disclosed herein, wherein the method comprises determining a hypoxia status of the ovarian cancer in the individual, and wherein the individual is selected if the individual has a hypoxia status indicative of the ovarian cancer or a portion thereof being hypoxic. In some embodiments, the hypoxia status of an ovarian cancer is based on one or more of the following: (i) a tissue oxygenation level; and (ii) a hypoxia biomarker. In some embodiments, the hypoxia status of an ovarian cancer is based on a low tissue oxygenation level. In some embodiments, the low tissue oxygenation level is a tissue oxygenation level of about 4% or less of oxygen, such as about 3% or less of oxygen, about 2% or less of oxygen, or about 1% or less of oxygen. In some embodiments, the tissue oxygenation level is based on an oxygenation level obtained via an oxymetric technique. In some embodiments, the hypoxia status of the ovarian cancer is determined based on one or more of the following: (i) assessing a tissue oxygenation level, such as via an oxymetric technique; and (ii) assessing a hypoxia biomarker. In some embodiments, the hypoxia status of an ovarian cancer is determined prior to administration of an effective amount of tirapazamine. In some embodiments, the methods disclosed herein further comprise determining a hypoxia status of an ovarian cancer prior to administration of an effective amount of tirapazamine. In some embodiments, the method further comprises selecting the individual for treatment based on the hypoxia status of the ovarian cancer. In some embodiments, the HR deficiency status of an ovarian cancer is further used as a basis for selecting the individual for treatment. In some embodiments, the IDH mutation status of an ovarian cancer is further used as a basis for selecting the individual for treatment.

In some embodiments, there is provided a method for treating a glioblastoma in an individual in need thereof, the method comprising administering to the individual an effective amount of tirapazamine, wherein an IDH mutation status of the glioblastoma is used as a basis for selecting the individual for treatment. In some embodiments, the present application provides methods for treating a glioblastoma in an individual having an IDH mutation in the glioblastoma or a portion thereof. In some embodiments, the method for treating a glioblastoma in an individual comprises selecting the individual for treatment based on an IDH mutation status, wherein the IDH mutation status is indicative of the glioblastoma comprising a mutation in IDH. In some embodiments, the present application provides methods of selecting (including identifying) an individual having a glioblastoma suitable for treatment with the methods disclosed herein, wherein the method comprises determining an IDH mutation status of the glioblastoma in the individual. In some embodiments, the present application provides methods of selecting (including identifying) an individual having a glioblastoma suitable for treatment with the methods disclosed herein, wherein the method comprises determining an IDH mutation status of the glioblastoma in the individual, and wherein the individual is selected if the IDH mutation status is indicative of the glioblastoma comprising a mutation in IDH. In some embodiments, the IDH mutation status of the glioblastoma is based on one or more of the following: (i) a gene sequence of an IDH isozyme; (ii) a change in activity of an IDH isozyme; and (iii) a level of a metabolic biomarker. In some embodiments, the IDH mutation status is based on an IDH mutation, such as one or more of an IDH1 mutation, IDH2 mutation, or IDH3 mutation. In some embodiments, the IDH mutation status of a glioblastoma is determined based on DNA sequencing of one or more genes, or a portion thereof. In some embodiments, the IDH mutation status of a glioblastoma is determined based on RNA sequencing of one or more genes transcripts, e.g., mRNA, or a portion thereof. In some embodiments, the IDH mutation status of a glioblastoma is determined based on protein sequencing of one or more gene products, or a portion thereof. In some embodiments, the IDH mutation status of a glioblastoma is determined based on one or more of the following: (i) assessing gene sequence, or product thereof, of an IDH isozyme; (ii) assessing a change in activity of an IDH isozyme; and (iii) assessing a level of a metabolic biomarker. In some embodiments, the IDH mutation status of a glioblastoma is determined prior to administration of an effective amount of tirapazamine. In some embodiments, the methods disclosed herein further comprise determining an IDH mutation status of a glioblastoma prior to administration of an effective amount of tirapazamine. In some embodiments, the methods disclosed herein further comprise selecting an individual for treatment based on an IDH mutation status of the glioblastoma. In some embodiments, the HR deficiency status of a glioblastoma is further used as a basis for selecting the individual for treatment. In some embodiments, the hypoxia status of a glioblastoma is further used as a basis for selecting the individual for treatment.

In some embodiments, there is provided a method for treating an acute myeloid leukemia (AML) in an individual in need thereof, the method comprising administering to the individual an effective amount of tirapazamine, wherein an IDH mutation status of the AML is used as a basis for selecting the individual for treatment. In some embodiments, the present application provides methods for treating an AML in an individual having an IDH mutation in the AML or a portion thereof. In some embodiments, the method for treating an AML in an individual comprises selecting the individual for treatment based on an IDH mutation status, wherein the IDH mutation status is indicative of the AML comprising a mutation in IDH. In some embodiments, the present application provides methods of selecting (including identifying) an individual having an AML suitable for treatment with the methods disclosed herein, wherein the method comprises determining an IDH mutation status of the AML in the individual. In some embodiments, the present application provides methods of selecting (including identifying) an individual having an AML suitable for treatment with the methods disclosed herein, wherein the method comprises determining an IDH mutation status of the AML in the individual, and wherein the individual is selected if the IDH mutation status is indicative of the AML comprising a mutation in IDH. In some embodiments, the IDH mutation status of the AML is based on one or more of the following: (i) a gene sequence of an IDH isozyme; (ii) a change in activity of an IDH isozyme; and (iii) a level of a metabolic biomarker. In some embodiments, the IDH mutation status is based on an IDH mutation, such as one or more of an IDH1 mutation, IDH2 mutation, or IDH3 mutation. In some embodiments, the IDH mutation status of an AML is determined based on DNA sequencing of one or more genes, or a portion thereof. In some embodiments, the IDH mutation status of an AML is determined based on RNA sequencing of one or more genes transcripts, e.g., mRNA, or a portion thereof. In some embodiments, the IDH mutation status of an AML is determined based on protein sequencing of one or more gene products, or a portion thereof. In some embodiments, the IDH mutation status of an AML is determined based on one or more of the following: (i) assessing gene sequence, or product thereof, of an IDH isozyme; (ii) assessing a change in activity of an IDH isozyme; and (iii) assessing a level of a metabolic biomarker. In some embodiments, the IDH mutation status of an AML is determined prior to administration of an effective amount of tirapazamine. In some embodiments, the methods disclosed herein further comprise determining an IDH mutation status of an AML prior to administration of an effective amount of tirapazamine. In some embodiments, the methods disclosed herein further comprise selecting an individual for treatment based on an IDH mutation status of the AML. In some embodiments, the HR deficiency status of an AML is further used as a basis for selecting the individual for treatment. In some embodiments, the hypoxia status of an AML is further used as a basis for selecting the individual for treatment.

In some embodiments, there is provided a method for treating a breast cancer in an individual in need thereof, the method comprising administering to the individual: (i) an effective amount of tirapazamine; and (ii) an effective amount of a PARP inhibitor, wherein the PARP inhibitor is selected from the group consisting of olaparib, talazoparib, and niraparib, wherein a HR deficiency status of the breast cancer is used as a basis for selecting the individual for treatment. In some embodiments, the method for treating a breast cancer in an individual comprises selecting the individual for treatment based on a positive status indicative of HR deficiency in the breast cancer or a portion thereof. In some embodiments, the present application provides methods of selecting (including identifying) an individual having a breast cancer suitable for treatment with the methods disclosed herein, wherein the method comprises determining a HR deficiency status of the breast cancer in the individual. In some embodiments, the present application provides methods of selecting (including identifying) an individual having a breast cancer suitable for treatment with the methods disclosed herein, wherein the method comprises determining a HR deficiency status of the breast cancer in the individual, and wherein the individual is selected if the individual has a positive status indicative of HR deficiency in the breast cancer or a portion thereof. In some embodiments, the HR deficiency status of the breast cancer is based on a HR deficiency signature. In some embodiments, the HR deficiency status of a breast cancer is based on one or more of the following: (i) a sequence of a gene or a product thereof; (ii) telomeric allelic imbalance (TAI); (iii) large-scale state transitions (LST); (iv) loss of heterozygosity (LOH); and (v) promoter methylation (or lack thereof). In some embodiments, the HR deficiency status of a breast cancer is determined based on DNA sequencing of one or more genes, or a portion thereof. In some embodiments, the HR deficiency status of a breast cancer is determined based on RNA sequencing of one or more genes transcripts, e.g., mRNA, or a portion thereof. In some embodiments, the HR deficiency status of a breast cancer is determined based on protein sequencing of one or more gene products, or a portion thereof. In some embodiments, the HR deficiency status of a breast cancer is determined based on one or more of the following: (i) assessing a gene sequence or a product thereof; (ii) assessing loss of heterozygosity (LOH); (iii) assessing telomeric allelic imbalance (TAI); (iv) assessing large-scale state transitions (LST); and (v) assessing promoter methylation (or lack thereof). In some embodiments, the HR deficiency status of the breast cancer is determined prior to administration of an effective amount of tirapazamine and/or a PARP inhibitor. In some embodiments, the methods disclosed herein further comprise determining a HR deficiency status of a breast cancer prior to administration of an effective amount of tirapazamine and/or a PARP inhibitor. In some embodiments, the methods disclosed herein further comprise selecting an individual for treatment based on a HR deficiency status of a breast cancer. In some embodiments, the IDH mutation status of a breast cancer is further used as a basis for selecting the individual for treatment. In some embodiments, the hypoxia status of a breast cancer is further used as a basis for selecting the individual for treatment.

In some embodiments, there is provided a method for treating a breast cancer in an individual in need thereof, the method comprising administering to the individual: (i) an effective amount of tirapazamine; and (ii) an effective amount of a PARP inhibitor, wherein the PARP inhibitor is selected from the group consisting of olaparib, talazoparib, and niraparib, wherein a hypoxia status of the breast cancer is used as a basis for selecting the individual for treatment. In some embodiments, the present application provides methods for treating a breast cancer in an individual having hypoxia in the breast cancer or a portion thereof. In some embodiments, the method for treating a breast cancer in an individual comprises selecting the individual for treatment based on a hypoxia status indicative of the breast cancer or a portion thereof being hypoxic. In some embodiments, the present application provides methods of selecting (including identifying) an individual having a breast cancer suitable for treatment with the methods disclosed herein, wherein the method comprises determining a hypoxia status of the breast cancer in the individual. In some embodiments, the present application provides methods of selecting (including identifying) an individual having a breast cancer suitable for treatment with the methods disclosed herein, wherein the method comprises determining a hypoxia status of the breast cancer in the individual, and wherein the individual is selected if the individual has a hypoxia status indicative of the breast cancer or a portion thereof being hypoxic. In some embodiments, the hypoxia status of a breast cancer is based on one or more of the following: (i) a tissue oxygenation level; and (ii) a hypoxia biomarker. In some embodiments, the hypoxia status of a breast cancer is based on a low tissue oxygenation level. In some embodiments, the low tissue oxygenation level is a tissue oxygenation level of about 4% or less of oxygen, such as about 3% or less of oxygen, about 2% or less of oxygen, or about 1% or less of oxygen. In some embodiments, the tissue oxygenation level is based on an oxygenation level obtained via an oxymetric technique. In some embodiments, the hypoxia status of the breast cancer is determined based on one or more of the following: (i) assessing a tissue oxygenation level, such as via an oxymetric technique; and (ii) assessing a hypoxia biomarker. In some embodiments, the hypoxia status of a breast cancer is determined prior to administration of an effective amount of tirapazamine and/or a PARP inhibitor. In some embodiments, the methods disclosed herein further comprise determining hypoxia status of a breast cancer prior to administration of an effective amount of tirapazamine and/or a PARP inhibitor. In some embodiments, the method further comprises selecting the individual for treatment based on the hypoxia status of the breast cancer. In some embodiments, the HR deficiency status of a breast cancer is further used as a basis for selecting the individual for treatment. In some embodiments, the IDH mutation status of a breast cancer is further used as a basis for selecting the individual for treatment.

In some embodiments, there is provided a method for treating an ovarian cancer in an individual in need thereof, the method comprising administering to the individual: (i) an effective amount of tirapazamine; and (ii) an effective amount of a PARP inhibitor, wherein the PARP inhibitor is selected from the group consisting of olaparib, talazoparib, and niraparib, wherein a HR deficiency status of the ovarian cancer is used as a basis for selecting the individual for treatment. In some embodiments, the method for treating an ovarian cancer in an individual comprises selecting the individual for treatment based on a positive status indicative of HR deficiency in the ovarian cancer or a portion thereof. In some embodiments, the present application provides methods of selecting (including identifying) an individual having an ovarian cancer suitable for treatment with the methods disclosed herein, wherein the method comprises determining a HR deficiency status of the ovarian cancer in the individual. In some embodiments, the present application provides methods of selecting (including identifying) an individual having an ovarian cancer suitable for treatment with the methods disclosed herein, wherein the method comprises determining a HR deficiency status of the ovarian cancer in the individual, and wherein the individual is selected if the individual has a positive status indicative of HR deficiency in the ovarian cancer or a portion thereof. In some embodiments, the HR deficiency status of the ovarian cancer is based on a HR deficiency signature. In some embodiments, the HR deficiency status of an ovarian cancer is based on one or more of the following: (i) a sequence of a gene or a product thereof; (ii) telomeric allelic imbalance (TAI); (iii) large-scale state transitions (LST); (iv) loss of heterozygosity (LOH); and (v) promoter methylation (or lack thereof). In some embodiments, the HR deficiency status of an ovarian cancer is determined based on DNA sequencing of one or more genes, or a portion thereof. In some embodiments, the HR deficiency status of an ovarian cancer is determined based on RNA sequencing of one or more genes transcripts, e.g., mRNA, or a portion thereof. In some embodiments, the HR deficiency status of an ovarian cancer is determined based on protein sequencing of one or more gene products, or a portion thereof. In some embodiments, the HR deficiency status of an ovarian cancer is determined based on one or more of the following: (i) assessing a gene sequence or a product thereof; (ii) assessing loss of heterozygosity (LOH); (iii) assessing telomeric allelic imbalance (TAI); (iv) assessing large-scale state transitions (LST); and (v) assessing promoter methylation (or lack thereof). In some embodiments, the HR deficiency status of the ovarian cancer is determined prior to administration of an effective amount of tirapazamine and/or a PARP inhibitor. In some embodiments, the methods disclosed herein further comprise determining a HR deficiency status of an ovarian cancer prior to administration of an effective amount of tirapazamine and/or a PARP inhibitor. In some embodiments, the methods disclosed herein further comprise selecting an individual for treatment based on a HR deficiency status of an ovarian cancer. In some embodiments, the IDH mutation status of an ovarian cancer is further used as a basis for selecting the individual for treatment. In some embodiments, the hypoxia status of an ovarian cancer is further used as a basis for selecting the individual for treatment.

In some embodiments, there is provided a method for treating an ovarian cancer in an individual in need thereof, the method comprising administering to the individual: (i) an effective amount of tirapazamine; and (ii) an effective amount of a PARP inhibitor, wherein the PARP inhibitor is selected from the group consisting of olaparib, talazoparib, and niraparib, wherein a hypoxia status of the ovarian cancer is used as a basis for selecting the individual for treatment. In some embodiments, the present application provides methods for treating an ovarian cancer in an individual having hypoxia in the ovarian cancer or a portion thereof. In some embodiments, the method for treating an ovarian cancer in an individual comprises selecting the individual for treatment based on a hypoxia status indicative of the ovarian cancer or a portion thereof being hypoxic. In some embodiments, the present application provides methods of selecting (including identifying) an individual having an ovarian cancer suitable for treatment with the methods disclosed herein, wherein the method comprises determining a hypoxia status of the ovarian cancer in the individual. In some embodiments, the present application provides methods of selecting (including identifying) an individual having an ovarian cancer suitable for treatment with the methods disclosed herein, wherein the method comprises determining a hypoxia status of the ovarian cancer in the individual, and wherein the individual is selected if the individual has a hypoxia status indicative of the ovarian cancer or a portion thereof being hypoxic. In some embodiments, the hypoxia status of an ovarian cancer is based on one or more of the following: (i) a tissue oxygenation level; and (ii) a hypoxia biomarker. In some embodiments, the hypoxia status of an ovarian cancer is based on a low tissue oxygenation level. In some embodiments, the low tissue oxygenation level is a tissue oxygenation level of about 4% or less of oxygen, such as about 3% or less of oxygen, about 2% or less of oxygen, or about 1% or less of oxygen. In some embodiments, the tissue oxygenation level is based on an oxygenation level obtained via an oxymetric technique. In some embodiments, the hypoxia status of the ovarian cancer is determined based on one or more of the following: (i) assessing a tissue oxygenation level, such as via an oxymetric technique; and (ii) assessing a hypoxia biomarker. In some embodiments, the hypoxia status of an ovarian cancer is determined prior to administration of an effective amount of tirapazamine and/or a PARP inhibitor. In some embodiments, the methods disclosed herein further comprise determining a hypoxia status of an ovarian cancer prior to administration of an effective amount of tirapazamine and/or a PARP inhibitor. In some embodiments, the method further comprises selecting the individual for treatment based on the hypoxia status of the ovarian cancer. In some embodiments, the HR deficiency status of an ovarian cancer is further used as a basis for selecting the individual for treatment. In some embodiments, the IDH mutation status of an ovarian cancer is further used as a basis for selecting the individual for treatment.

In some embodiments, there is provided a method for treating a glioblastoma in an individual in need thereof, the method comprising administering to the individual: (i) an effective amount of tirapazamine; and (ii) an effective amount of a PARP inhibitor, wherein the PARP inhibitor is selected from the group consisting of olaparib, talazoparib, and niraparib, wherein an IDH mutation status of the glioblastoma is used as a basis for selecting the individual for treatment. In some embodiments, the present application provides methods for treating a glioblastoma in an individual having an IDH mutation in the glioblastoma or a portion thereof. In some embodiments, the method for treating a glioblastoma in an individual comprises selecting the individual for treatment based on an IDH mutation status, wherein the IDH mutation status is indicative of the glioblastoma comprising a mutation in IDH. In some embodiments, the present application provides methods of selecting (including identifying) an individual having a glioblastoma suitable for treatment with the methods disclosed herein, wherein the method comprises determining an IDH mutation status of the glioblastoma in the individual. In some embodiments, the present application provides methods of selecting (including identifying) an individual having a glioblastoma suitable for treatment with the methods disclosed herein, wherein the method comprises determining an IDH mutation status of the glioblastoma in the individual, and wherein the individual is selected if the IDH mutation status is indicative of the glioblastoma comprising a mutation in IDH. In some embodiments, the IDH mutation status of the glioblastoma is based on one or more of the following: (i) a gene sequence of an IDH isozyme; (ii) a change in activity of an IDH isozyme; and (iii) a level of a metabolic biomarker. In some embodiments, the IDH mutation status is based on an IDH mutation, such as one or more of an IDH1 mutation, IDH2 mutation, or IDH3 mutation. In some embodiments, the IDH mutation status of a glioblastoma is determined based on DNA sequencing of one or more genes, or a portion thereof. In some embodiments, the IDH mutation status of a glioblastoma is determined based on RNA sequencing of one or more genes transcripts, e.g., mRNA, or a portion thereof. In some embodiments, the IDH mutation status of a glioblastoma is determined based on protein sequencing of one or more gene products, or a portion thereof. In some embodiments, the IDH mutation status of a glioblastoma is determined based on one or more of the following: (i) assessing gene sequence, or product thereof, of an IDH isozyme; (ii) assessing a change in activity of an IDH isozyme; and (iii) assessing a level of a metabolic biomarker. In some embodiments, the IDH mutation status of a glioblastoma is determined prior to administration of an effective amount of tirapazamine and/or a PARP inhibitor. In some embodiments, the methods disclosed herein further comprise determining an IDH mutation status of a glioblastoma prior to administration of an effective amount of tirapazamine and/or a PARP inhibitor. In some embodiments, the methods disclosed herein further comprise selecting an individual for treatment based on an IDH mutation status of the glioblastoma. In some embodiments, the HR deficiency status of a glioblastoma is further used as a basis for selecting the individual for treatment. In some embodiments, the hypoxia status of a glioblastoma is further used as a basis for selecting the individual for treatment.

In some embodiments, there is provided a method for treating an acute myeloid leukemia (AML) in an individual in need thereof, the method comprising administering to the individual: (i) an effective amount of tirapazamine; and (ii) an effective amount of a PARP inhibitor, wherein the PARP inhibitor is selected from the group consisting of olaparib, talazoparib, and niraparib, wherein an IDH mutation status of the AML is used as a basis for selecting the individual for treatment. In some embodiments, the present application provides methods for treating an AML in an individual having an IDH mutation in the AML or a portion thereof. In some embodiments, the method for treating an AML in an individual comprises selecting the individual for treatment based on an IDH mutation status, wherein the IDH mutation status is indicative of the AML comprising a mutation in IDH. In some embodiments, the present application provides methods of selecting (including identifying) an individual having an AML suitable for treatment with the methods disclosed herein, wherein the method comprises determining an IDH mutation status of the AML in the individual. In some embodiments, the present application provides methods of selecting (including identifying) an individual having an AML suitable for treatment with the methods disclosed herein, wherein the method comprises determining an IDH mutation status of the AML in the individual, and wherein the individual is selected if the IDH mutation status is indicative of the AML comprising a mutation in IDH. In some embodiments, the IDH mutation status of the AML is based on one or more of the following: (i) a gene sequence of an IDH isozyme; (ii) a change in activity of an IDH isozyme; and (iii) a level of a metabolic biomarker. In some embodiments, the IDH mutation status is based on an IDH mutation, such as one or more of an IDH1 mutation, IDH2 mutation, or IDH3 mutation. In some embodiments, the IDH mutation status of an AML is determined based on DNA sequencing of one or more genes, or a portion thereof. In some embodiments, the IDH mutation status of an AML is determined based on RNA sequencing of one or more genes transcripts, e.g., mRNA, or a portion thereof. In some embodiments, the IDH mutation status of an AML is determined based on protein sequencing of one or more gene products, or a portion thereof. In some embodiments, the IDH mutation status of an AML is determined based on one or more of the following: (i) assessing gene sequence, or product thereof, of an IDH isozyme; (ii) assessing a change in activity of an IDH isozyme; and (iii) assessing a level of a metabolic biomarker. In some embodiments, the IDH mutation status of an AML is determined prior to administration of an effective amount of tirapazamine and/or a PARP inhibitor. In some embodiments, the methods disclosed herein further comprise determining an IDH mutation status of an AML prior to administration of an effective amount of tirapazamine and/or a PARP inhibitor. In some embodiments, the methods disclosed herein further comprise selecting an individual for treatment based on an IDH mutation status of the AML. In some embodiments, the HR deficiency status of an AML is further used as a basis for selecting the individual for treatment. In some embodiments, the hypoxia status of an AML is further used as a basis for selecting the individual for treatment.

### Kits, Medicines, and Compositions

The present disclosure, in some aspects, also provides kits, medicines, and compositions, for use in any of the methods described herein.

Kits of the present disclosure include one or more containers comprising a hypoxia targeting composition (such as a hypoxia-activated drug or a prodrug thereof) (or a unit dosage and/or an article of manufacture thereof). In some embodiments, the kit further comprises one or more containers comprising another agent (or a unit dosage and/or an article of manufacture thereof), such as a PARP inhibitor. In some embodiments, the kit further comprises instructions for use in accordance with any of the methods disclosed herein. The kit may also comprise a description of criteria for selection of an individual suitable for treatment with any of the methods disclosed herein. Instructions supplied in the kits disclosed herein are typically written instructions on a label or package insert (e.g., a paper sheet included in the kit), but machine-readable instructions (e.g., instructions carried on a magnetic or optical storage disk) are also acceptable.

For example, in some embodiments, the kit comprises: (a) one or more containers comprising a hypoxia targeting composition (such as a hypoxia-activated drug or a prodrug thereof) (or a unit dosage and/or an article of manufacture thereof); and (b) instructions for selecting an individual for a treatment of a cancer with the hypoxia targeting composition (such as the hypoxia-activated drug or the prodrug thereof), wherein a HR deficiency status of the cancer is used as a basis for selecting the individual for the treatment. In some embodiments, the kit further comprises instructions for administering to an individual a hypoxia activated drug or a prodrug thereof. In some embodiments, the kit further comprises instructions and/or components (such as reagents) for assessing a HR deficiency status in an individual.

In some embodiments, the kit comprises: (a) one or more containers comprising a hypoxia targeting composition (such as a hypoxia-activated drug or a prodrug thereof) (or a unit dosage and/or an article of manufacture thereof); and (b) instructions for selecting an individual for a treatment of a cancer with the hypoxia targeting composition (such as the hypoxia-activated drug or the prodrug thereof), wherein an IDH mutation status of the cancer is used as a basis for selecting the individual for the treatment. In some embodiments, the kit further comprises instructions for administering to an individual a hypoxia activated drug or a prodrug thereof. In some embodiments, the kit further comprises instructions and/or components (such as reagents) for assessing an IDH mutation status in an individual.

In some embodiments, the kit comprises: (a) one or more containers comprising a hypoxia targeting composition (such as a hypoxia-activated drug or a prodrug thereof) (or a unit dosage and/or an article of manufacture thereof); and (b) instructions for selecting an individual for a treatment of a cancer with the hypoxia targeting composition (such as the hypoxia-activated drug or the prodrug thereof), wherein a hypoxia status of the cancer is used as a basis for selecting the individual for the treatment. In some embodiments, the kit further comprises instructions for administering to an individual a hypoxia activated drug or a prodrug thereof. In some embodiments, the kit further comprises instructions and/or components (such as reagents) for assessing a hypoxia status in an individual.

In some embodiments, the kit comprises: (a) one or more containers comprising a hypoxia targeting composition (such as a hypoxia-activated drug or a prodrug thereof) (or a unit dosage and/or an article of manufacture thereof); and (b) instructions for selecting an individual for a treatment of a cancer with the hypoxia targeting composition (such as the hypoxia-activated drug or the prodrug thereof), wherein one or more of a HR deficiency status, an IDH mutation status, and a hypoxia status of the cancer is used as a basis for selecting the individual for the treatment. In some embodiments, the kit further comprises instructions for administering to an individual a hypoxia activated drug or a prodrug thereof. In some embodiments, the kit further comprises instructions and/or components (such as reagents) for assessing one or more of a HR deficiency status, an IDH mutation status, and a hypoxia status in an individual.

In some embodiments, the kit comprises: (a) one or more containers comprising a hypoxia targeting composition (such as a hypoxia-activated drug or a prodrug thereof) (or a unit dosage and/or an article of manufacture thereof); and (b) one or more containers comprising a PARP inhibitor (or a unit dosage and/or an article of manufacture thereof). In some embodiments, the kit further comprises instructions for selecting an individual for a treatment of a cancer with the combination of (a) the hypoxia targeting composition (such as the hypoxia-activated drug or the prodrug thereof), and (b) the PARP inhibitor, wherein a HR deficiency status of the cancer is used as a basis for selecting the individual for the treatment. In some embodiments, the kit further comprises instructions for selecting an individual for a treatment of a cancer with the combination of (a) the hypoxia targeting composition (such as the hypoxia-activated drug or the prodrug thereof), and (b) the PARP inhibitor, wherein an IDH mutation status of the cancer is used as a basis for selecting the individual for the treatment. In some embodiments, the kit further comprises instructions for selecting an individual for a treatment of a cancer with the combination of (a) the hypoxia targeting composition (such as the hypoxia-activated drug or the prodrug thereof), and (b) the PARP inhibitor, wherein a hypoxia status of the cancer is used as a basis for selecting the individual for the treatment. In some embodiments, the kit further comprises instructions for administering to an individual a combination of (a) a hypoxia targeting composition (such as a hypoxia-activated drug or a prodrug thereof), and (b) a PARP inhibitor. In some embodiments, the kit further comprises instructions and/or components (such as reagents) for assessing a HR deficiency status in an individual. In some embodiments, the kit further comprises instructions and/or components (such as reagents) for assessing an IDH mutation status in an individual. In some embodiments, the kit further comprises instructions and/or components (such as reagents) for assessing a hypoxia status in an individual.

In some embodiments, the hypoxia targeting composition (such as the hypoxia-activated drug or the prodrug thereof) may be present in separate containers or in a single container. In some embodiments, the PARP inhibitor may be present in separate containers or in a single container. In some embodiments, the hypoxia targeting composition (such as the hypoxia-activated drug or the prodrug thereof) and the PARP inhibitor may be present in separate containers or in a single container.

In some embodiments, the kits of the present disclosure are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

In some embodiments, instructions relating to the use of a hypoxia targeting composition (such as a hypoxia-activated drug or a prodrug thereof) and/or a PARP inhibitor generally include information as to dosage, dosing schedule, and route of administration for the intended treatment. The containers may be unit doses, bulk packages (e.g., multi-dose packages) or sub-unit doses. For example, kits may be provided that contain sufficient dosages of a hypoxia targeting composition (such as a hypoxia-activated drug or a prodrug thereof) and/or a PARP inhibitor to provide effective treatment, as disclosed herein, of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses comprising a hypoxia targeting composition (such as a hypoxia-activated drug or a prodrug thereof) and/or a PARP inhibitor and instructions for use and packaged in quantities sufficient for storage and use in pharmacies, for example, hospital pharmacies and compounding pharmacies.

Also provided in the present disclosure are medicines and compositions (such as unit dosages) useful for the methods described herein. For example, in some embodiments, there is provided use of a hypoxia targeting composition (such as a hypoxia-activated drug or a prodrug thereof) for a treatment of a cancer in an individual in need thereof, wherein a HR deficiency status of the cancer is used as a basis for selecting the individual for the treatment. In some embodiments, there is provided a hypoxia targeting composition (such as a hypoxia-activated drug or a prodrug thereof) for the manufacture of a medicament for a treatment of a cancer, wherein a HR deficiency status of the cancer is used as a basis for selecting the individual for the treatment. In some embodiments, there is provided a hypoxia targeting composition (such as a hypoxia-activated drug or a prodrug thereof) for the manufacture of a medicament for a treatment of a HR deficient cancer.

In some embodiments, there is provided use of a hypoxia targeting composition (such as a hypoxia-activated drug or a prodrug thereof) for a treatment of a cancer in an individual in need thereof, wherein an IDH mutation status of the cancer is used as a basis for selecting the individual for the treatment. In some embodiments, there is provided a hypoxia targeting composition (such as a hypoxia-activated drug or a prodrug thereof) for the manufacture of a medicament for a treatment of a cancer, wherein an IDH mutation status of the cancer is used as a basis for selecting the individual for the treatment. In some embodiments, there is provided a hypoxia targeting composition (such as a hypoxia-activated drug or a prodrug thereof) for the manufacture of a medicament for a treatment of an IDH mutant cancer (such as a cancer comprising an IDH mutation).

In some embodiments, there is provided use of a hypoxia targeting composition (such as a hypoxia-activated drug or a prodrug thereof) for a treatment of a cancer in an individual in need thereof, wherein a hypoxia status of the cancer is used as a basis for selecting the individual for the treatment. In some embodiments, there is provided a hypoxia targeting composition (such as a hypoxia-activated drug or a prodrug thereof) for the manufacture of a medicament for a treatment of a cancer, wherein a hypoxia status of the cancer is used as a basis for selecting the individual for the treatment. In some embodiments, there is provided a hypoxia targeting composition (such as a hypoxia-activated drug or a prodrug thereof) for the manufacture of a medicament for a treatment of a hypoxic cancer (such as a cancer comprising at least a portion thereof with a low oxygen concentration, e.g. less than about 2% oxygen concentration).

In some embodiments, there is provided use of: (a) a hypoxia targeting composition (such as a hypoxia-activated drug or a prodrug thereof); and (b) a PARP inhibitor, for a treatment of a cancer in an individual in need thereof. In some embodiments, there is provided use of: (a) a hypoxia targeting composition (such as a hypoxia-activated drug or a prodrug thereof); and (b) a PARP inhibitor, for a treatment of a cancer in an individual in need thereof, wherein one or more of a HR deficiency status, an IDH mutation status, and a hypoxia status of the cancer is used as a basis for selecting the individual for the treatment. In some embodiments, there is provided: (a) a hypoxia targeting composition (such as a hypoxia-activated drug or a prodrug thereof); and (b) a PARP inhibitor, for the manufacture of a medicament combination for a treatment of a cancer. In some embodiments, there is provided: (a) a hypoxia targeting composition (such as a hypoxia-activated drug or a prodrug thereof); and (b) a PARP inhibitor, for the manufacture of a medicament combination for a treatment of a cancer, wherein one or more of a HR deficiency status, an IDH mutation status, and a hypoxia status of the cancer is used as a basis for selecting the individual for the treatment. In some embodiments, there is provided: (a) a hypoxia targeting composition (such as a hypoxia-activated drug or a prodrug thereof); and (b) a PARP inhibitor, for the manufacture of a medicament combination for a treatment of a HR deficient cancer. In some embodiments, there is provided: (a) a hypoxia targeting composition (such as a hypoxia-activated drug or a prodrug thereof); and (b) a PARP inhibitor, for the manufacture of a medicament combination for a treatment of an IDH mutant cancer (such as a cancer comprising an IDH mutation). In some embodiments, there is provided: (a) a hypoxia targeting composition (such as a hypoxia-activated drug or a prodrug thereof); and (b) a PARP inhibitor, for the manufacture of a medicament combination for a treatment of a hypoxic cancer (such as a cancer comprising at least a portion thereof with a low oxygen concentration, e.g. less than about 2% oxygen concentration).

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example demonstrates that cell lines deficient for homologous recombination (HR) cultured in hypoxic conditions show insensitivity to treatments with PARP inhibitors. This example also demonstrates that a combination of a hypoxia-activated drug or a prodrug thereof plus a PARP inhibitor resulted in significant toxicity both in vitro and in vivo, as compared to single agent treatments and a vehicle control.

HR Deficient Cell Lines Cultured in Hypoxic Conditions Show Insensitivity to Treatments with a PARP Inhibitor

The effect of oxygen concentrations on PARP inhibitor induced toxicity or reduction in cell survival for HR deficient cell lines was assessed. Specifically, a series of cell lines deficient for HR were treated with PARP inhibitors (olaparib or talazoparib (BMN 673)) for 7 days in 21% oxygen (representative of normoxic conditions) or 2% oxygen (representative of hypoxic conditions). In duplicates, 250-2000 cells of each cell line were seeded in 6-well plates and treated with varying concentrations of olaparib (0-1 μM) or talazoparib (0-10 nM). At the end of the treatment, media from each well was changed to drug-free media and cells were incubated to allow for formation of colonies. At the end of each incubation, colonies were fixed in 70% ethanol and stained with Crystal Violet to facilitate counting of colonies. At least 3 biological replicates were performed for each condition tested. The HR deficient cell lines treated were SUM149 (a BRAC1 mutant, triple negative breast cancer cell line), CAPAN-1 (a BRAC2 mutant, pancreatic cancer cell line), HT1080 (an IDH mutant, fibrosarcoma cell line), and OVCAR8 (a hypermethylated BRAC1 promoter, ovarian serous adenocarcinoma (high grade) cell line).

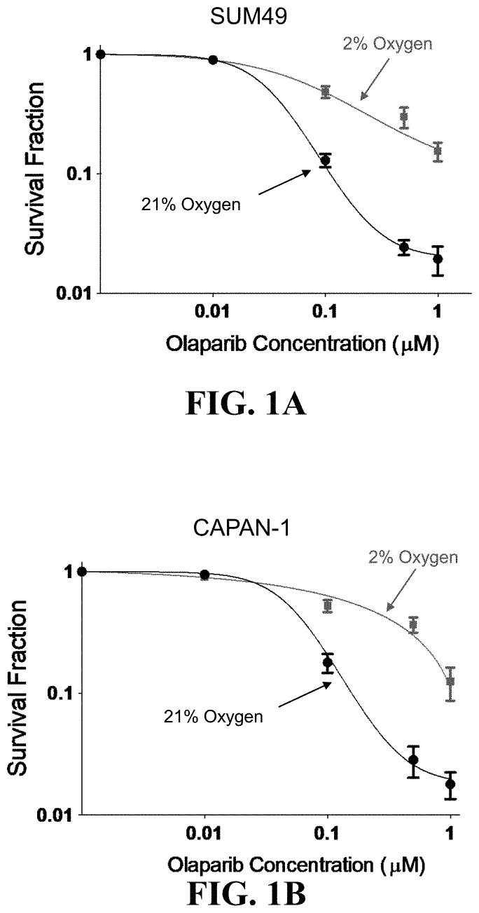

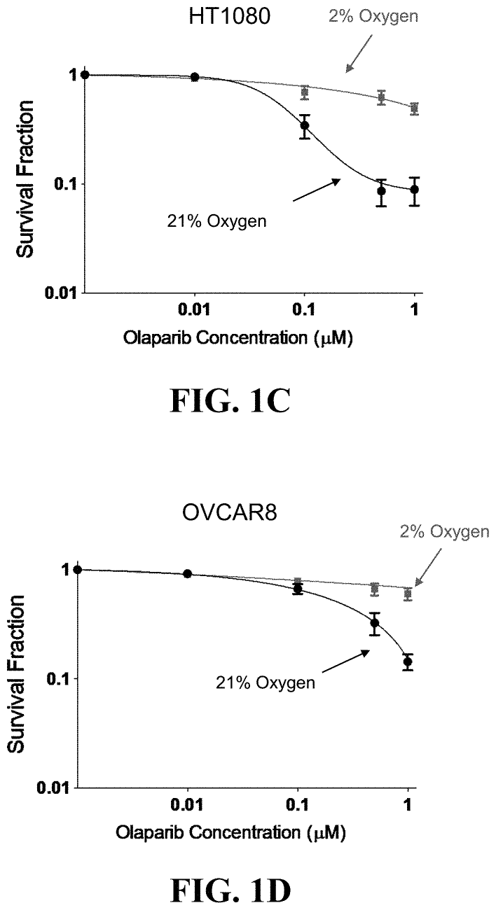

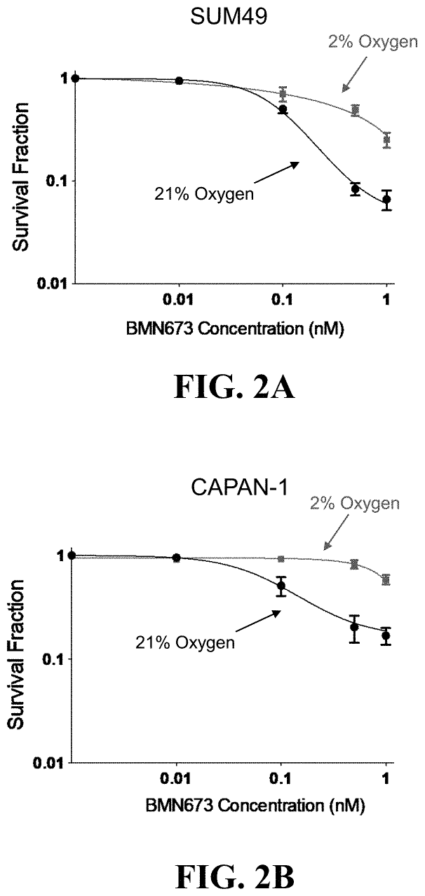

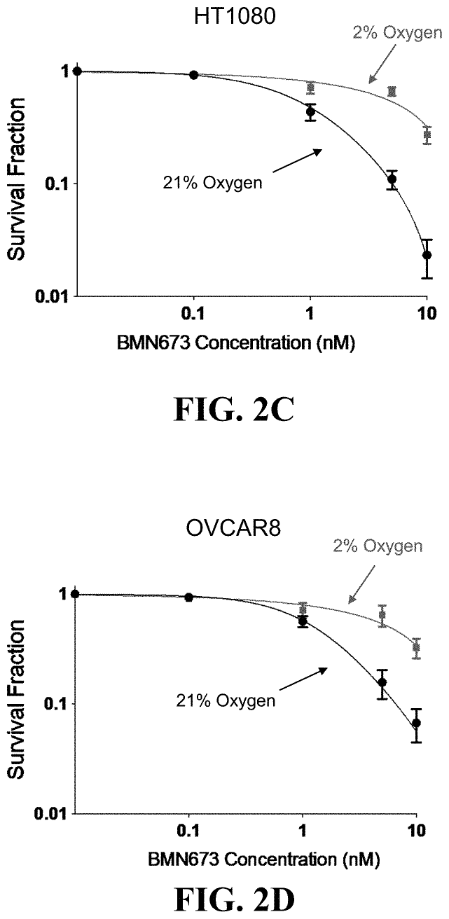

As shown in FIGS. 1A-1D and FIGS. 2A-2D, the survival fractions of HR deficient cell line samples, when cultured at 21% oxygen concentrations, were reduced over the concentration spectra of PARP inhibitors, olaparib and talazoparib. In contrast, the HR deficient cell line samples cultured at 2% oxygen demonstrated a trend of higher survival fractions over the PARP inhibitor concentrations as compared to correspondingly treated cell line samples cultured at 21% oxygen. These data demonstrated that at hypoxic conditions, namely, 2% oxygen concentration, the HR deficient cell lines showed insensitivity to PARP inhibitor induced toxicity or reduction in cell survival than when cultured at higher, non-hypoxic oxygen concentrations.

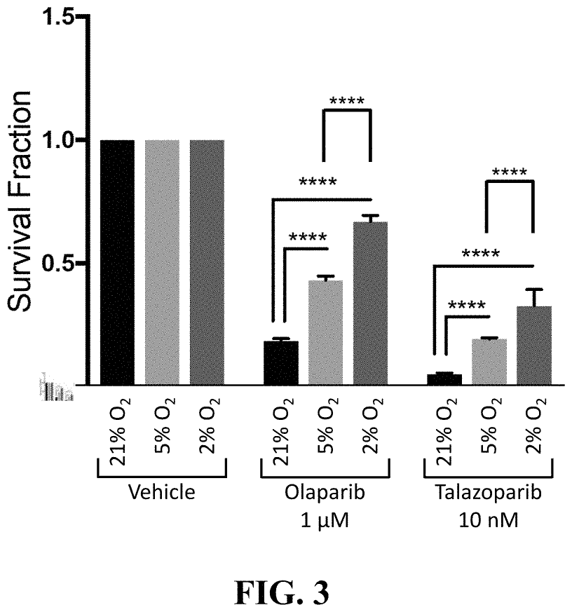

These observations were further evaluated using the OVCAR8 cell line samples at 21% oxygen, 5% oxygen, and 2% oxygen concentrations. Specifically, OVCAR8 cell line samples were cultured in 21% oxygen, 5% oxygen, or 2% oxygen conditions and were treated with a vehicle, olaparib (1 μM), or talazoparib (BMN 673; 10 nM) for 7 days. At the end of the treatment, media from each well was changed to a drug-free media and cells were incubated to allow for formation colonies. As shown in FIG. 3, OVCAR8 cell line samples cultured at 5% oxygen and 2% oxygen concentrations each showed significantly improved survival in the presence of PARP inhibitors as compared to correspondingly treated cell line samples cultured at 21% oxygen conditions. Additionally, OVCAR8 cell line samples cultured at a 2% oxygen concentration showed significantly improved survival in the presence of PARP inhibitors as compared to correspondingly treated cell line samples cultured at a 5% oxygen concentration.

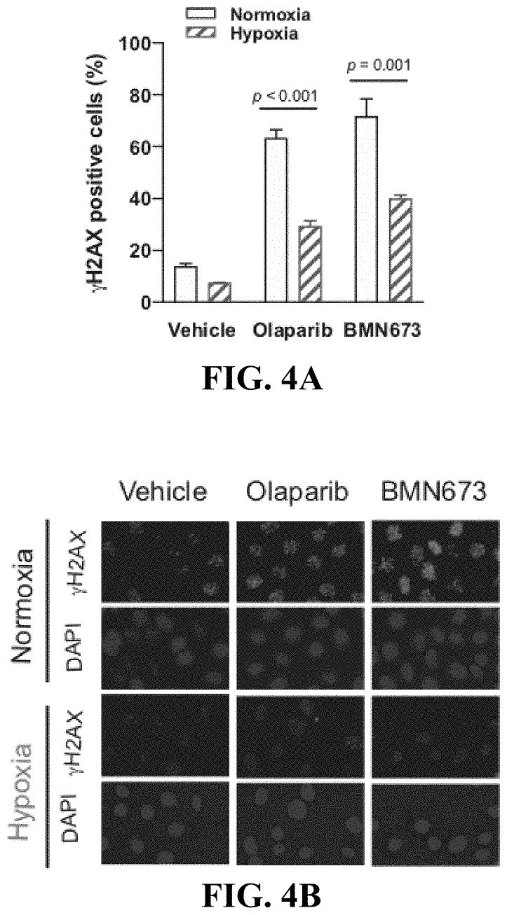

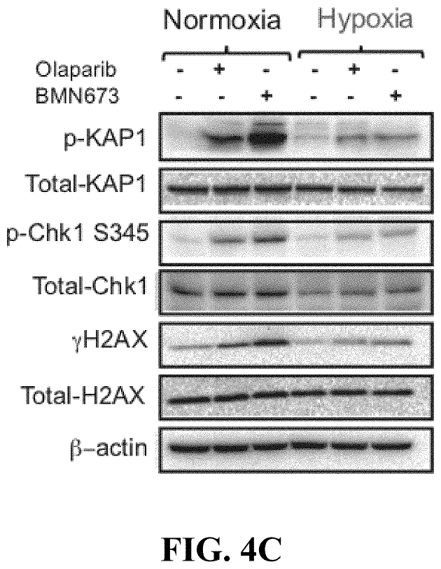

DNA damage foci studies were performed in SUM149 cells which revealed substantially increased γH2AX foci in normoxia (21% oxygen) as compared to hypoxia (2% oxygen) upon inhibition of PARP (FIG. 4A). Representative microscopy images are shown in FIG. 4B. PARPi induced a significant amount of DNA damage signaling as detected by Western blot analyses in normoxic but not hypoxic cells in both SUM149 (FIG. 4C). Treatment with PARPi for 48 h in normoxia was associated with a strong induction of phosphorylation DDR markers γH2AX, KAP1, Chk1, while phosphorylated levels of these proteins were significantly lower in PARPi-treated hypoxic cells.

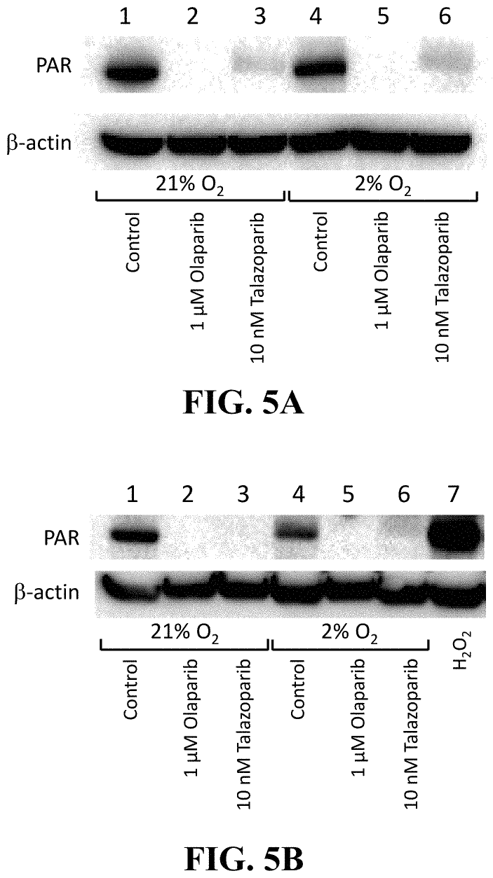

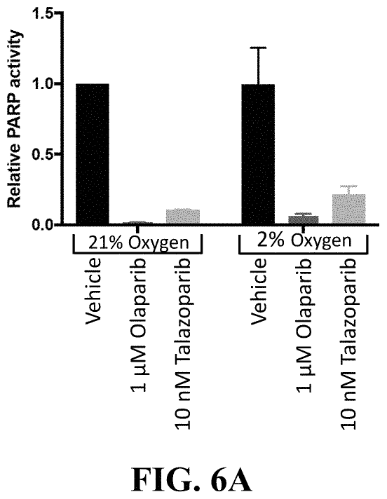

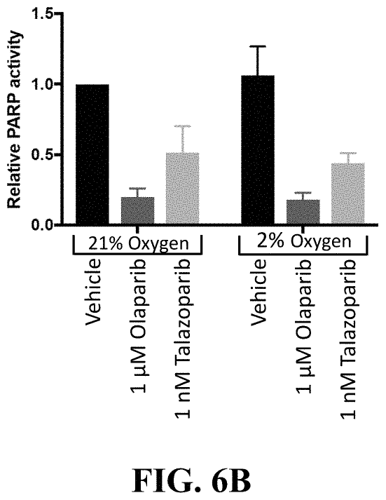

To further investigate the observed insensitivity to PARP inhibitors observed in HR deficient cell lines cultured at low oxygen concentrations, PARP enzyme activity (using formation of poly(ADP-ribose) (PAR) as a surrogate) following treatment with a PARP inhibitor was measured at 21% oxygen and 2% oxygen conditions to understand if there is a differential ability of PARP inhibitors to inhibit PARP enzyme activity at different oxygen conditions. Western blot analysis of PAR formation was conducted in OVCAR8 cells and HT1080 cells following treatment with a vehicle, olaparib (AZD-2281; 1 μM), or talazoparib (BMN 673; 10 nM) cultured in 21% oxygen or 2% oxygen conditions for 7 days. PAR levels in cell lysates were quantified using western blot. As shown in FIGS. 5A and 5B, olaparib and talazoparib reduced PAR formation in both 21% oxygen and 2% oxygen conditions, indicating that PARP inhibitors inhibit PARP enzyme activity independent of cellular oxygen conditions, such as a hypoxic environment. As further shown in FIGS. 6A and 6B, relative PARP activities in OVCAR8 cells (FIG. 6A) and in SUM149 cells (FIG. 6B) were significantly reduced upon treatment with a PARP inhibitor under both 21% oxygen and 2% oxygen conditions.

The observed PARP inhibitor insensitivity in hypoxic HR deficient cells was further evaluated in vivo. OVCAR8 xenografts were established by subcutaneous injection of tumor cells in serum-free media/matrigel mix. Once tumors reached a size of about 100 mm³, animals were divided into the following groups: (i) vehicle (10% 2-hydroxy-propyl-β-cyclodextrin/PBS) once a day via intraperitoneal injection (ii) olaparib (50 mg/kg) administered daily via intraperitoneal injection for 2 days. 3 animals were used per group.

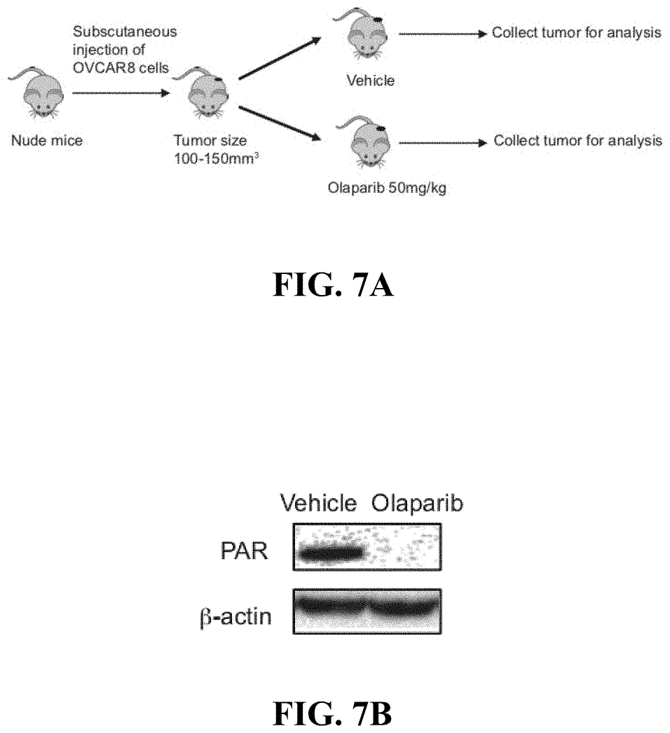

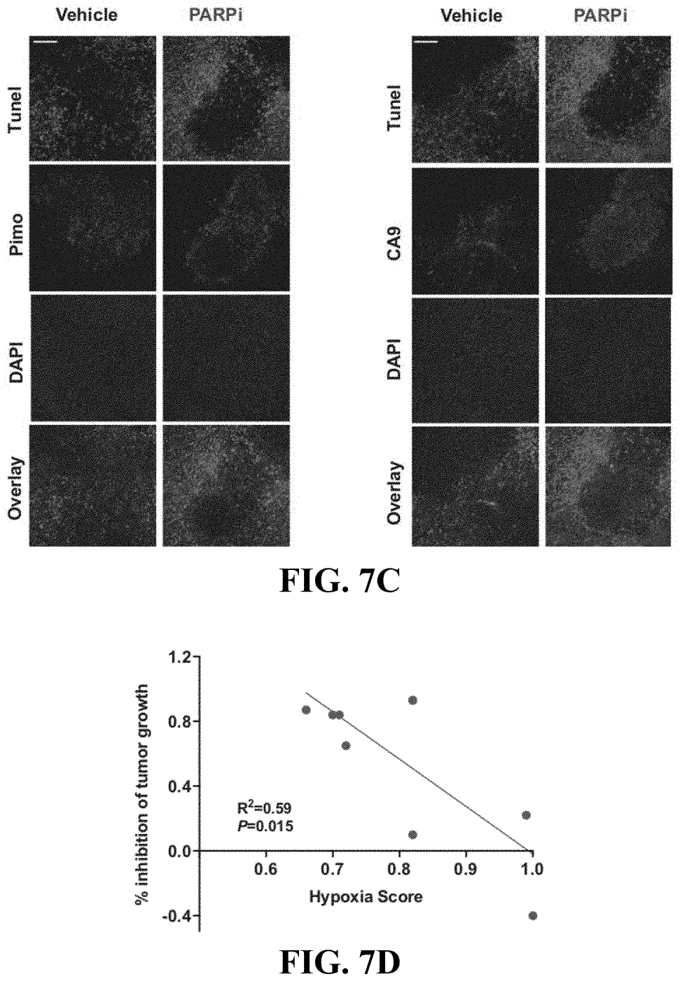

Following completion of each treatment regimen, tumors were collected for analysis (FIG. 7A). PARP enzyme activity (measured via formation of PAR) was evaluated using western blot analysis. As shown in FIG. 7B, no evidence of PAR formation was observed in the xenograft samples from olaparib treatment. In contrast, the vehicle treatment group had PAR formation, indicating enzymatic activity of PARP in the OVCAR8 xenograft.

Tissue slices from harvested tumors were then analyzed using pimonidazole or CA9 (hypoxia marker), Dapi (DNA), and Tunel (marker of apoptosis) (FIG. 7C). Immunohistochemical staining confirmed decreased expression of Tunel in hypoxic (pimonidazole or CA9 positive) tumor subregions of PARP-treated tumors as compared to non-hypoxic regions, confirming that hypoxia is associated with resistance to PARP inhibition in vivo. These results demonstrate that PARP inhibitor insensitivity in hypoxic HR deficient cells also occurs in vivo.

Efficacy of olaparib in a range of patient-derived tumor xenografts (PDTXs) were previously tested in vivo by Bruna et al and the results archived in a biobank (http://caldaslab.cruk.cam.ac.uk/bcape) (see Bruna et al. Cell 167, 260-274 (2016), the disclosure of which is incorporated herein by reference). The efficacy of olaparib in these breast PDTX models in relation to their hypoxia levels was analyzed. A previously validated, robust hypoxia signature, developed by Buffa et al was utilized, revealing a strong inverse correlation between hypoxia score of the tumors and their sensitivity to olaparib therapy, where tumors with higher hypoxic score were the most resistant to PARPi therapy (FIG. 7D). (Buffa et al. Br J Cancer 102, 428-35 (2010), the disclosure of which is incorporated herein by reference). This is in accordance with the data from in vitro studies.

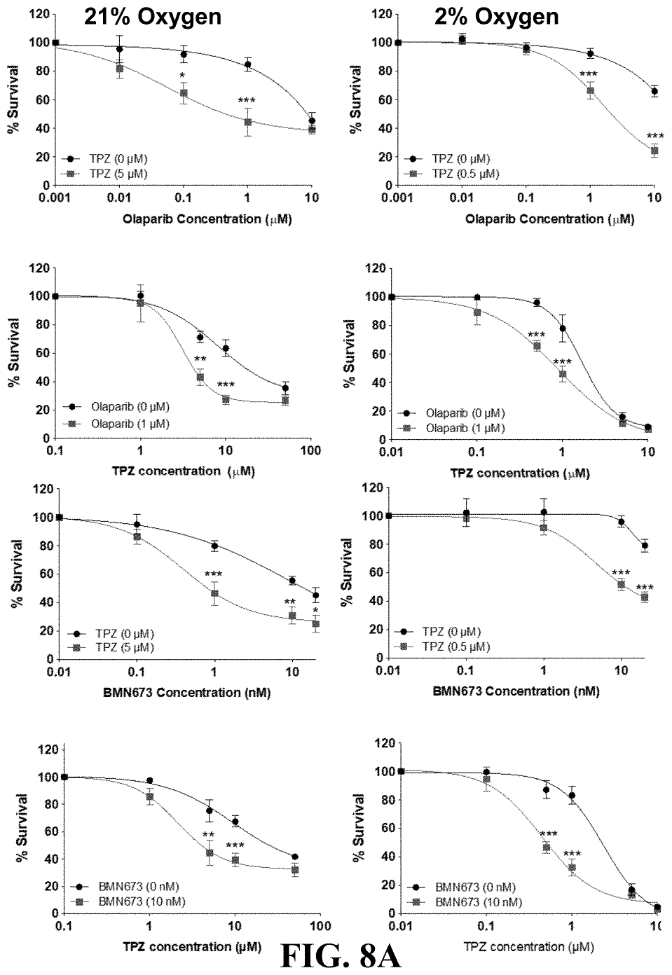

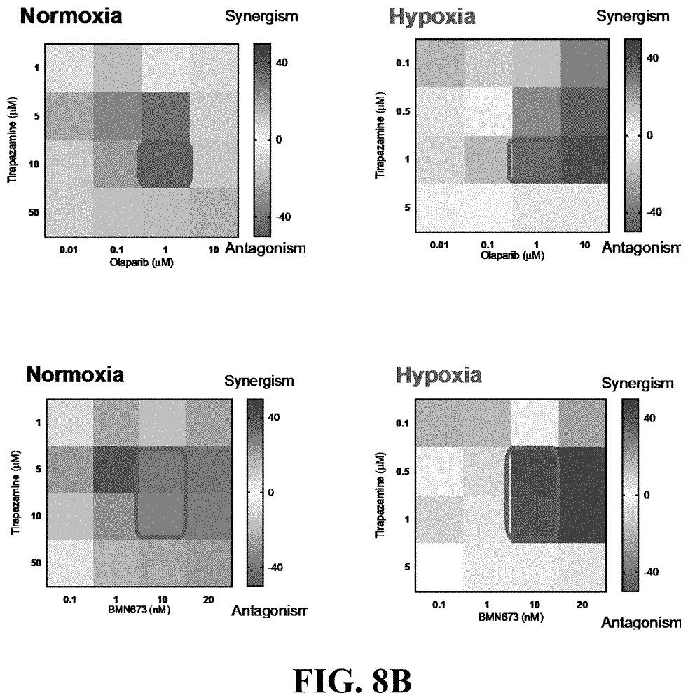

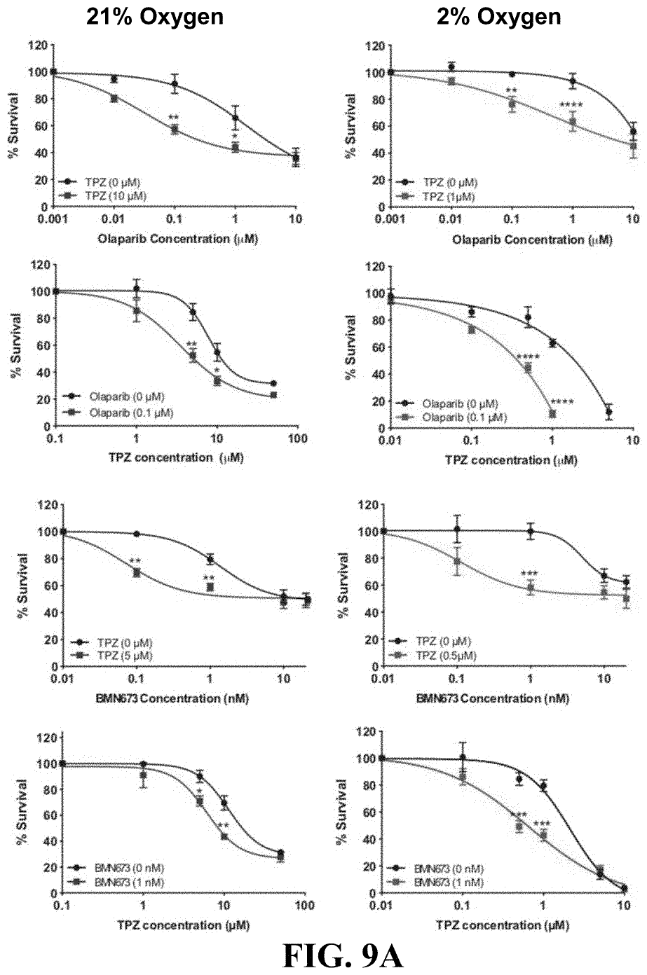

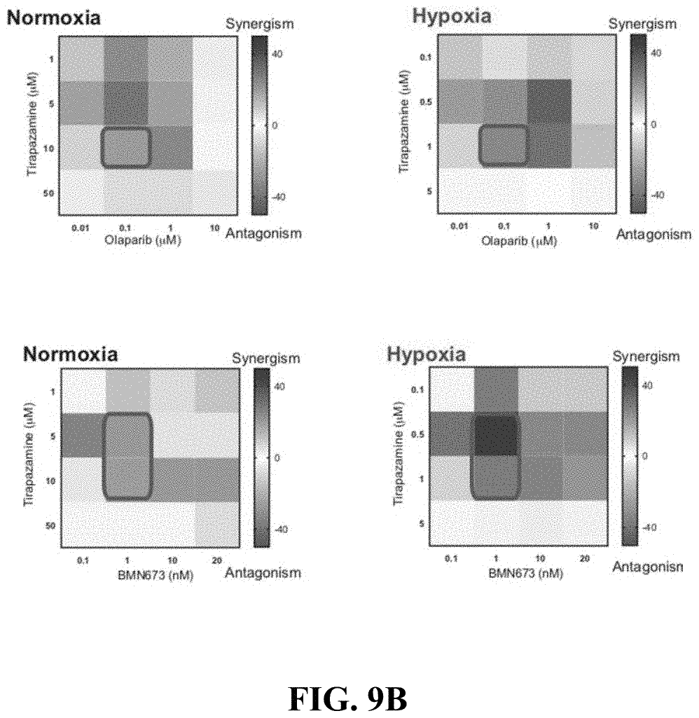

The effect of combining a hypoxia activated prodrug with PARPi on cellular toxicity was assessed. Tirapazamine is a hypoxia activated prodrug that is currently used in clinical trials, and combining this drug with PARPi in OVCAR8 and SUM149 cells resulted in a substantial decrease in cell survival as compared to either drug alone (FIG. 8A, 8B, 9A & 9B). Combenefit; a validated, open-access software program was used to analyze the data and quantify possible synergistic or antagonistic drug interactions, and relative cell kill data are presented as survival curves (FIG. 8A& 9A), while synergy scores are presented in matrix format (FIG. 8B& 9B). Using a classical Lowe synergy model, a substantial synergistic interaction between tirapazamine and PARPi (olaparib and BMN673) was detected, which was stronger under hypoxic conditions with 10-fold lower doses of tirapazamine required to achieve similar or even higher synergy scores to normoxic conditions. Examples are highlighted with the red squares in the matrix plots. In OVCAR8 cells for example, tirapazamine doses required to achieve a synergy score of 30-40 when cells are treated with 10 nM BMN673 are 0.5-1 μM in hypoxia and 5-10 μM in normoxia. A similar trend was observed with the OVCAR8 cells where a synergy score of 20-30 requires treatment with 0.1 μM olaparib are 1 μM in hypoxia and 10 μM in normoxia.

Significant Reduction of Tumor Growth with a Combination Comprising a Hypoxia-Activated Drug or a Prodrug Thereof and a PARP Inhibitor

The in vitro efficacy of single agent tirapazamine or a single agent PARP inhibitor (olaparib or talazoparib) or combinations of tirapazamine and a PARP inhibitor (olaparib or talazoparib) was assessed in HR deficient cell lines (OVCAR8 and SUM149) at 21% oxygen and 2% oxygen concentrations. Specifically, OVCAR8 cells were seeded and treated with one of the following for 96 hours in 21% oxygen or 2% oxygen concentrations: (i) vehicle; (ii) tirapazamine (TPZ; 0.1 μM or 1 μM); (iii) olaparib (OL; 1 μM); (iv) tirapazamine (at concentrations above) plus olaparib (1 μM); (v) talazoparib (BMN; 10 nM); or tirapazamine (at concentrations above) plus talazoparib (10 nM). SUM149 cells were seeded and treated with one of the following for 96 hours in 21% oxygen or 2% oxygen concentrations: (i) vehicle; (ii) tirapazamine (TPZ; 0.1 μM or 1 μM); (iii) olaparib (OL; 0.1 μM); (iv) tirapazamine (at concentrations above) plus olaparib (0.1 μM); (v) talazoparib (BMN; 1 nM); or tirapazamine (at concentrations above) plus talazoparib (1 nM). At the end of the treatment, media from each sample was changed to a drug-free media and cells were incubated to allow for formation colonies before survival fraction was determined.

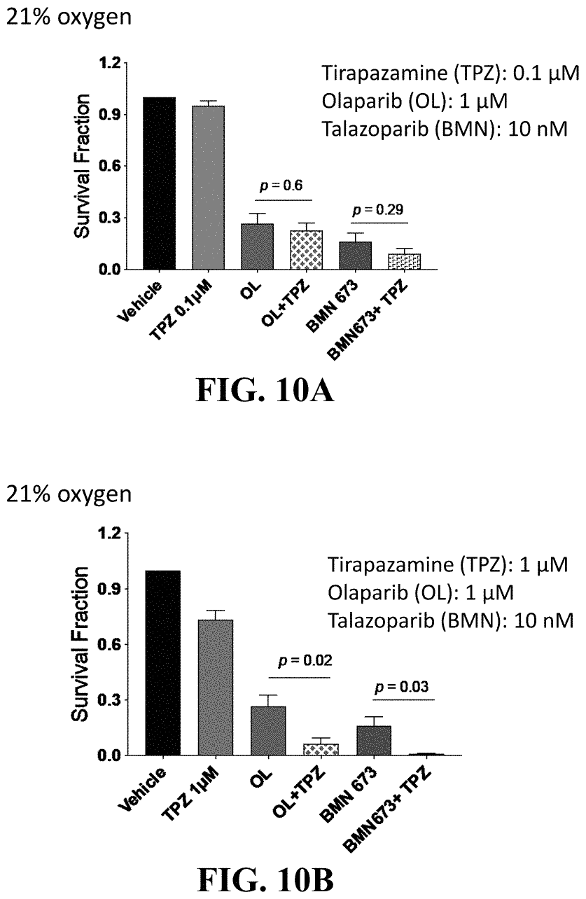

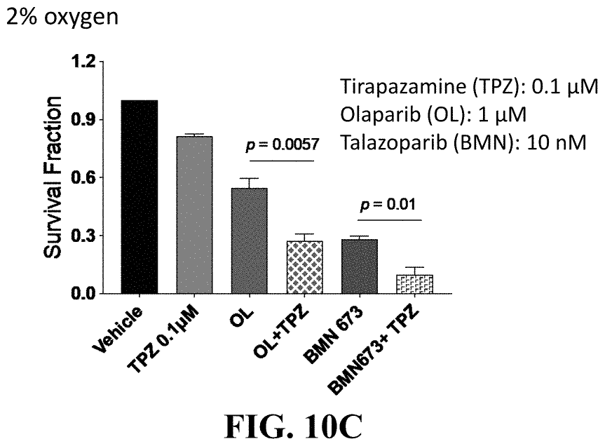

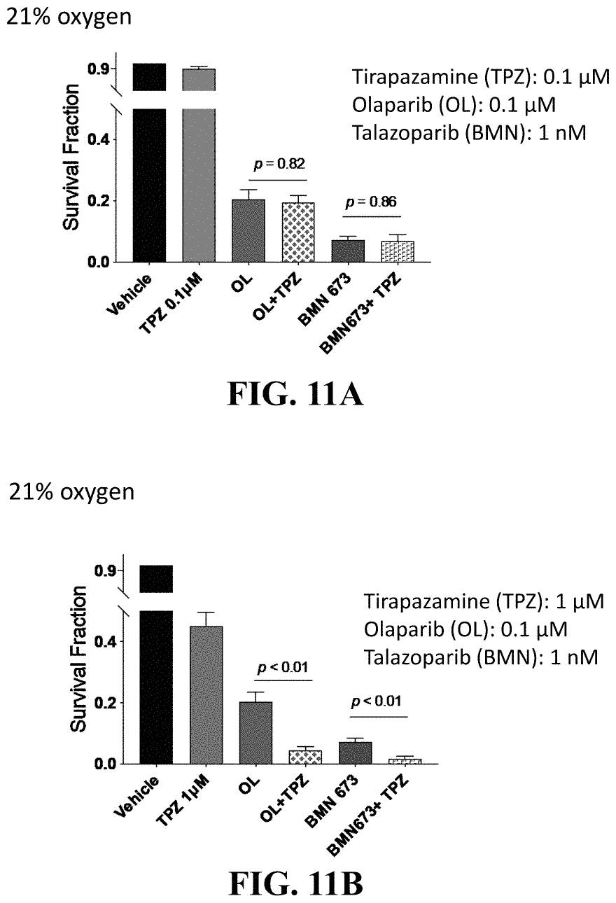

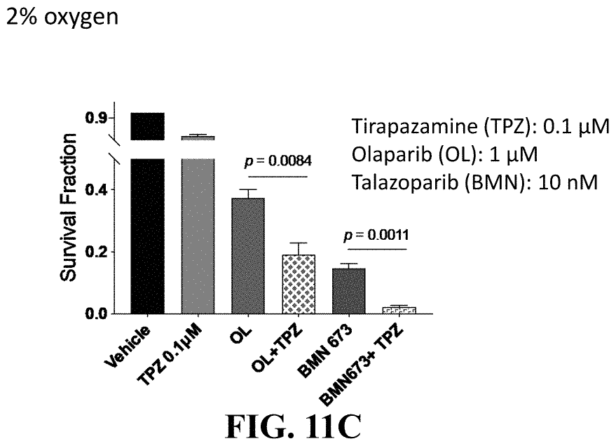

As shown in FIGS. 10A-10C and FIGS. 11A-11C, for both OVCAR8 and SUM149 cell lines, tirapazamine did not have a significant effect on cell survival fraction as compared to the vehicle. For both OVCAR8 and SUM149 cell lines, at 21% oxygen concentration, no increase in reducing cell survival fraction for the combination of tirapazamine (0.1 μM) and a PARP inhibitor was observed. Interestingly, for both OVCAR8 and SUM149 cell lines, there was enhanced reduction in cell survival fraction for the combination of tirapazamine (0.1 μM) and a PARP inhibitor (olaparib or talazoparib) when the cells were cultured in a 2% oxygen condition. Additionally, for cells cultured in 21% oxygen conditions, increased reduction in cell survival fraction for the combination of tirapazamine and a PARP inhibitor was observed when the dosage of tirapazamine was increased to 1 μM.

The in vivo efficacy of a combination of tirapazamine and a PARP inhibitor (olaparib or talazoparib) was assessed in HR deficient xenografts (OVCAR8 or SUM149). OVCAR8 xenografts were generated as discussed above. Once tumors reached a size of about 100 mm³, animals were randomized and divided into 4 groups according to the following treatments (5 animals per group): (i) vehicle, (ii) olaparib (50 mg/kg) administered daily via intraperitoneal injection 5 times a week; (iii) tirapazamine (20 mg/kg) administered via intraperitoneal injection every 2-3 days; and (iv) olaparib plus tirapazamine (doses as in single treatment groups). Treatment of the animals continued until the end of the study.

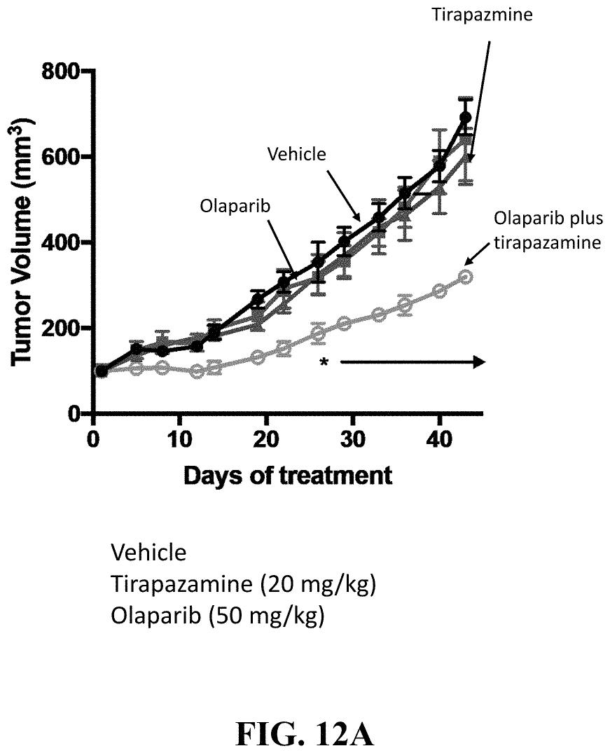

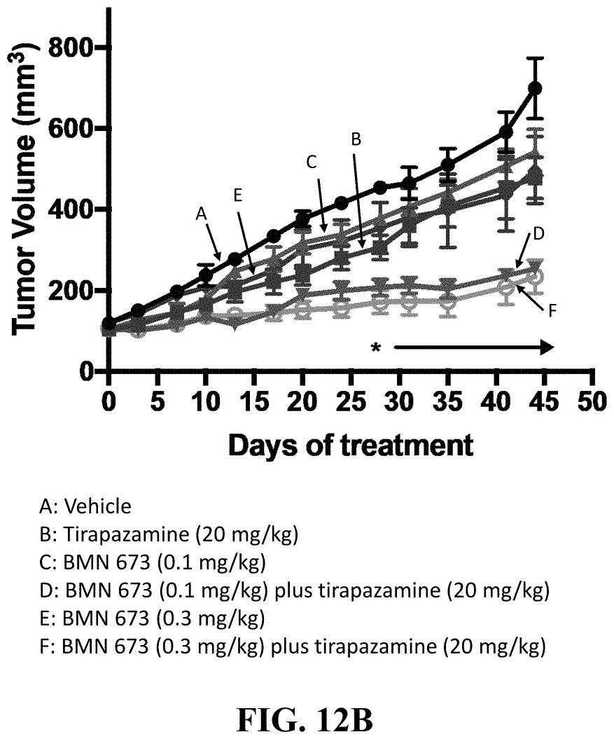

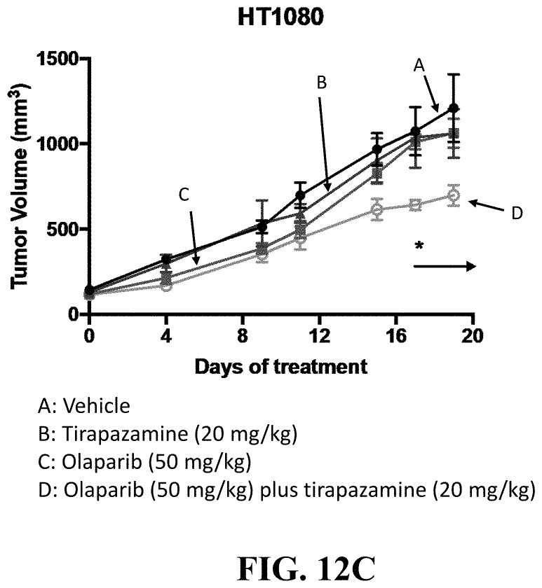

As shown in FIG. 12A, the combination treatment of olaparib plus tirapazamine significantly delayed tumor growth as compared to treatments with a vehicle, single agent olaparib, and single agent tirapazamine.

SUM149 xenografts were established by subcutaneous injection of tumor cells in serum-free media/matrigel mix. Once tumors reached a size of about 100 mm³, animals were treated according to their assignment to one of the following groups: (A) vehicle, (B) tirapazamine (20 mg/kg) administered via intraperitoneal injection every 5 days; (C) talazoparib (BMN 673; 0.1 mg/kg) administered daily 5 times a week by oral gavage; (D) tirapazamine (20 mg/kg) administered via intraperitoneal injection every 5 days plus talazoparib (0.1 mg/kg) administered daily 5 times a week by oral gavage; (E) talazoparib (BMN 673; 0.3 mg/kg) administered daily 5 times a week by oral gavage; and (F) tirapazamine (20 mg/kg) administered via intraperitoneal injection every 5 days plus talazoparib (0.3 mg/kg) administered daily 5 times a week by oral gavage.

As shown in FIG. 12B, the treatment combination of tirapazamine plus talazoparib significantly delayed SUM149 tumor growth at both concentrations studied (groups D and F) as compared to treatments with a vehicle, single agent talazoparib, and single agent tirapazamine.

The in vivo efficacy of a combination of tirapazamine and a PARP inhibitor (olaparib) was assessed in a HT1080 xenograft (IDH mutant, fibrosarcoma cell line). HT1080 xenografts were generated in a similar manner as discussed above. Once tumors reached a size of about 100 mm³, animals were randomized and divided into 4 groups according to the following treatments (5 animals per group): (A) vehicle, (B) tirapazamine (20 mg/kg) administered via intraperitoneal injection every 2-3 days; (C) olaparib (50 mg/kg) administered daily via intraperitoneal injection 5 times per week; and (D) olaparib plus tirapazamine (doses as in single treatment groups). Treatment of the animals continued until the end of the study.

As shown in FIG. 12C, the combination treatment of olaparib plus tirapazamine significantly delayed tumor growth as compared to treatments with a vehicle, single agent olaparib, and single agent tirapazamine.

### DOCTRINE OF EQUIVALENTS

In particular, as can be inferred from the above discussion, the above mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

1. A method for treating a cancer in an individual in need thereof, the method comprising administering to the individual (i) an effective amount of a hypoxia targeting composition, and (ii) an effective amount of a poly(ADP-ribose) polymerase (PARP) inhibitor.

2. The method of claim 1, wherein the hypoxia targeting composition is a hypoxia-activated drug or a prodrug thereof.

3. The method of claim 2, wherein the hypoxia-activated drug or the prodrug thereof is selected from the group consisting of: apaziquone, AQ4N, etanidazole, evofosfamide, nimorazole, pimonidazole, porfiromycin, PR-104, tarloxotinib, and tirapazamine, or an analog or derivative thereof.

4. The methods of claim 1, wherein the effective amount of the hypoxia targeting composition is about 0.1 mg to 1000 mg.

5. The method of claim 1, wherein the effective amount of the hypoxia targeting composition is suitable for oral administration.

6. The method of claim 1, wherein the PARP inhibitor is selected from the group consisting of: 3-aminobenzamine, BGD-290, CEP 9722, E7016, iniparib, niraparib, olaparib, rucaparib, talazoparib, Fluzoparib, and veliparib.

7. The method of claim 1, wherein the effective amount of the PARP inhibitor is about 20 mg to about 2000 mg.

8.-9. (canceled)

10. The method of claim 1, wherein (i) the effective amount of the hypoxia targeting composition, and (ii) the effective amount of the PARP inhibitor are administered simultaneously, sequentially, or concurrently.

11.-12. (canceled)

13. The method of claim 1, wherein any one or more of an homologous recombination (HR) deficiency status, an IDH mutation status, or a hypoxia status of the cancer is used as a basis for selecting the individual for treatment.

14.-34. (canceled)

35. A method for treating a cancer in an individual in need thereof, the method comprising administering to the individual an effective amount of a hypoxia targeting composition, wherein a homologous recombination (HR) deficiency status, an IDH mutation status, or a hypoxia status of the cancer is used as a basis for selecting the individual for treatment.

36. The method of claim 35, wherein the hypoxia targeting composition is a hypoxia-activated drug or a prodrug thereof.

37. The method of claim 36, wherein the hypoxia-activated drug or the prodrug thereof is selected from the group consisting of: apaziquone, AQ4N, etanidazole, evofosfamide, nimorazole, pimonidazole, porfiromycin, PR-104, tarloxotinib, and tirapazamine, or an analog or derivative thereof.

38. The methods of claim 35, wherein the effective amount of the hypoxia targeting composition is about 0.1 mg to 1000 mg.

39. The method of claim 35, wherein the effective amount of the hypoxia-activated drug or the prodrug thereof is suitable for oral administration.

40. The method of claim 35, wherein the HR deficiency status of the cancer is based on a HR deficiency signature.

41. The method of claim 35, wherein the HR deficiency status of the cancer is based on one or more of the following: (i) a gene sequence, or a product thereof, or an expression level thereof; (ii) loss of heterozygosity (LOH); (iii) telomeric allelic imbalance (TAI); (iv) large-scale state transitions (LST); and (v) promoter methylation.

42. (canceled)

43. The method of claim 35, wherein the HR deficiency status of the cancer is based on one or more of: DNA sequencing, RNA sequencing, and protein sequencing.

44. The method of claim 35, wherein the HR deficiency status of the cancer is determined prior to administration of the effective amount of the hypoxia targeting composition.

45. The method of claim 35, further comprising determining the HR deficiency status of the cancer prior to administration of the effective amount of the hypoxia targeting composition.

46. The method of claim 35, further comprising selecting the individual for treatment based on the HR deficiency status of the cancer.

47.-51. (canceled)

52. The method of claim 35, wherein the IDH mutation status is based on an IDH mutation.

53. The method of claim 35, wherein the IDH mutation status of the cancer is based on one or more of the following: (i) a gene sequence, or a product thereof, of IDH1 and/or IDH2; (ii) a change in an activity level of IDH1 and/or IDH2; and (iii) a level of a metabolic biomarker.

54.-61. (canceled)

62. The method of claim 35, wherein the hypoxia status of the cancer is based on a low tissue oxygenation level.

63. The method of claim 62, wherein the low tissue oxygenation level is a tissue oxygenation level of about 4% or less of oxygen.

64. The method of claim 35, wherein the hypoxia status of the cancer is based on one or more of the following: (i) tissue oxygenation level; and (ii) a hypoxia biomarker.

65.-67. (canceled)

68. The method of claim 1, wherein the cancer is a solid tumor.

69. The method of claim 1, wherein the cancer is a hematopoietic malignancy.

70. The method of claim 1, wherein the cancer is a breast cancer, ovarian cancer, pancreatic cancer, fibrosarcoma, head and neck cancer, prostate cancer, glioma, or acute myeloid leukemia.

71. The method of claim 1, wherein the individual is human.

72. A kit comprising: (i) a hypoxia targeting composition, and (ii) a poly(ADP-ribose) polymerase (PARP) inhibitor.

73. (canceled)
